SEQUENCE LISTING (502 sequences)

SEQ ID NO: 1 - DNA, 4185 bp, Homo sapiens
  source 1..4185: mol_type = other DNA
attttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccaca

SEQ ID NO: 2 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccgcttcgctcccacaaaat

SEQ ID NO: 3 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acccaagcgcagcccagcca

SEQ ID NO: 4 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggacaccgaagcagcgacgg

SEQ ID NO: 5 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctgctgggaagcccgacag

SEQ ID NO: 6 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttacttttcccgctaggcc

SEQ ID NO: 7 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caccgtcccatcgtcttccg

SEQ ID NO: 8 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctgtaaccgtggagagcag

SEQ ID NO: 9 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacacgcccctggaaactgg

SEQ ID NO: 10 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tggattcctgtagcgaagcc

SEQ ID NO: 11 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctctcatacactgagacac

SEQ ID NO: 12 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttccttctaccagccggaca

SEQ ID NO: 13 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agcatctggggcatgcagaa

SEQ ID NO: 14 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tacaccagatttccccagcc

SEQ ID NO: 15 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttttggatagttgacaaca

SEQ ID NO: 16 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atccatttttcttttgttat

SEQ ID NO: 17 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctgatgaagcatctgtctc

SEQ ID NO: 18 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgctcttttcactttcact

SEQ ID NO: 19 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taaatcggatgttttctgga

SEQ ID NO: 20 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catgggagacccaacactat

SEQ ID NO: 21 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggtcctgttcggttgttttc

SEQ ID NO: 22 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggtactaaaatactctttca

SEQ ID NO: 23 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
accataagaacttctccaaa

SEQ ID NO: 24 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taagatctttcttgacctgc

SEQ ID NO: 25 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cccctttgaatgaccagtct

SEQ ID NO: 26 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtaaaacgaacaaagccaaa

SEQ ID NO: 27 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcacttgtgtttcatattcc

SEQ ID NO: 28 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgtgacattactttcacttg

SEQ ID NO: 29 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgtcgctgtgacattactt

SEQ ID NO: 30 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caccatcgtccatctatcat

SEQ ID NO: 31 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aattaggaagtttgcagtca

SEQ ID NO: 32 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcatcttggctttgcttag

SEQ ID NO: 33 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acttttctgcttctcaaagg

SEQ ID NO: 34 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgtacagcgccccacaaac

SEQ ID NO: 35 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcatcctcagtcatgtcct

SEQ ID NO: 36 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgagagaagaactcccgcag

SEQ ID NO: 37 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catccatcacatccccgtac

SEQ ID NO: 38 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaatggcttggggatgaaga

SEQ ID NO: 39 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtaacaaaggcaaaggccct

SEQ ID NO: 40 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caatctgatcatctgcaaat

SEQ ID NO: 41 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctctccacaaagagactgcg

SEQ ID NO: 42 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctattgtgcttaggttcgg

SEQ ID NO: 43 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttctttctaactgtctatt

SEQ ID NO: 44 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gattaccaccaaatcttcca

SEQ ID NO: 45 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaagccacctggattacca

SEQ ID NO: 46 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgattcccaaagccacctg

SEQ ID NO: 47 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgctattaccaaatccacc

SEQ ID NO: 48 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccaaaccagctccaccccct

SEQ ID NO: 49 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attactaccttgattgtttc

SEQ ID NO: 50 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagttcatcccaccacccat

SEQ ID NO: 51 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gattaatgctgaacgcacca

SEQ ID NO: 52 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggcggcagccatcatggctg

SEQ ID NO: 53 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgctctgtagtgctgcctg

SEQ ID NO: 54 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atcataccccaactgctctg

SEQ ID NO: 55 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acatgcccatcataccccaa

SEQ ID NO: 56 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctggttctgctggctggcta

SEQ ID NO: 57 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttattacccgatgggcctga

SEQ ID NO: 58 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcatgttgccttggttttgg

SEQ ID NO: 59 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggcctggtttggctccctct

SEQ ID NO: 60 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gagttatttccagaaccgaa

SEQ ID NO: 61 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cagaattagagccactataa

SEQ ID NO: 62 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tccccaaccaattgctgcac

SEQ ID NO: 63 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaccctgcattggatgctga

SEQ ID NO: 64 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctccattaaaaccactgccc

SEQ ID NO: 65 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atccatgcttgagccaaagc

SEQ ID NO: 66 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccccagccagaagacttaga

SEQ ID NO: 67 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acaaccccactgtctacatt

SEQ ID NO: 68 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccattctataccaaccaacc

SEQ ID NO: 69 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tagaaaaatttgaattccca

SEQ ID NO: 70 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttaccatgagtttagaaaa

SEQ ID NO: 71 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caatatacttaccatgagtt

SEQ ID NO: 72 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttagtacatatgtatttta

SEQ ID NO: 73 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acaaaccaattttgaaaatt

SEQ ID NO: 74 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgaatatactccacactga

SEQ ID NO: 75 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgtcaaaaatactgctgaa

SEQ ID NO: 76 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaaaaatgtcaaaaatactg

SEQ ID NO: 77 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tagctcttcctttttctaaa

SEQ ID NO: 78 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaaacttataaaattcctt

SEQ ID NO: 79 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatttcaacctttcatgtaa

SEQ ID NO: 80 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gttcactttcaaccactcaa

SEQ ID NO: 81 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taccaatcaggcaaacagca

SEQ ID NO: 82 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcaattgtagtgtgttggtt

SEQ ID NO: 83 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acaggagaaaccttttgata

SEQ ID NO: 84 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagtccagggataaaatatt

SEQ ID NO: 85 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgcaaagaattcacttgac

SEQ ID NO: 86 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caatggtttccgttttgaac

SEQ ID NO: 87 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtaaagaatgtagttctaat

SEQ ID NO: 88 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggttcaaattaaaacaaggg

SEQ ID NO: 89 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaaaaaaatccatatggtgg

SEQ ID NO: 90 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taaaaggagattttcttaag

SEQ ID NO: 91 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
actgtgacaccatgatctcc

SEQ ID NO: 92 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acaaaacaaaagaaccaaac

SEQ ID NO: 93 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgagatgaactgcagagat

SEQ ID NO: 94 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgaaatgagatgaactgca

SEQ ID NO: 95 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agtgcttcttccataaacat

SEQ ID NO: 96 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cactactttcaatgaagtgc

SEQ ID NO: 97 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tacagcactactttcaatga

SEQ ID NO: 98 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tattcctatggcagaatatt

SEQ ID NO: 99 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgagaaagcatgtagacag

SEQ ID NO: 100 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcgtgatgacgaattcttga

SEQ ID NO: 101 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcaaagacgcggcctgtgat

SEQ ID NO: 102 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taaaaatgggacacccaccg

SEQ ID NO: 103 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagagtagcggataaaaatg

SEQ ID NO: 104 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgaaataaagagtagcgga

SEQ ID NO: 105 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catagcgttgatacgactcc

SEQ ID NO: 106 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cagccttgcgttcatagcgt

SEQ ID NO: 107 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccatatcacagccttgcgtt

SEQ ID NO: 108 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gttcagacagccttctggtt

SEQ ID NO: 109 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcccacacaaggtttcaaaa

SEQ ID NO: 110 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgcctcggcaccaccatcaa

SEQ ID NO: 111 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
actagcctttcatgcctcgg

SEQ ID NO: 112 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcgctcatactagcctttca

SEQ ID NO: 113 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcacgcgctctctccttttc

SEQ ID NO: 114 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
accaccaagtctctgcacgc

SEQ ID NO: 115 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttatgcaccaccaagtctct

SEQ ID NO: 116 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gccaagttaaaaaatatcca

SEQ ID NO: 117 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aggattgagagacacatctc

SEQ ID NO: 118 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cactctctcaccaaagccac

SEQ ID NO: 119 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cattgctctctgcacactct

SEQ ID NO: 120 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgctatcattgctctctgca

SEQ ID NO: 121 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaaacattcgtacattatt

SEQ ID NO: 122 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtggatgtcctttgaatgca

SEQ ID NO: 123 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttaaaagtcttccaacagat

SEQ ID NO: 124 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atctaagaacaaaaactcac

SEQ ID NO: 125 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acattcatctaatgtgggtt

SEQ ID NO: 126 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caagtatcatttcacttaac

SEQ ID NO: 127 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acaaaggggtagggggagta

SEQ ID NO: 128 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tacagcattcacagcagttg

SEQ ID NO: 129 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacaccatacagcattcaca

SEQ ID NO: 130 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acagaagagaacacacacca

SEQ ID NO: 131 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gccacacttacatatcagta

SEQ ID NO: 132 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catcagcttcagttcacatt

SEQ ID NO: 133 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcagtccatgttctcagcc

SEQ ID NO: 134 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccacaagctcagtccatgtt

SEQ ID NO: 135 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctgcaaagcacaccacaag

SEQ ID NO: 136 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgaactctgcttcaagtcct

SEQ ID NO: 137 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gagacacctgagctcactgg

SEQ ID NO: 138 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tagaacttccacccttcttt

SEQ ID NO: 139 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatgggtagctaacagacat

SEQ ID NO: 140 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
actgcagcaaacagcattct

SEQ ID NO: 141 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacagaactgcagcaaacag

SEQ ID NO: 142 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atccaagcacaggacacaga

SEQ ID NO: 143 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgacaactcttataaaaagc

SEQ ID NO: 144 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aacaatgacaactcttataa

SEQ ID NO: 145 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatttaagaatttccaacaa

SEQ ID NO: 146 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catattatttaaatcagttt

SEQ ID NO: 147 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaacaaagacacatattat

SEQ ID NO: 148 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agggctgcaaaacaaagaca

SEQ ID NO: 149 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctatgaattctttgcattc

SEQ ID NO: 150 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
taactgctatgaattctttg

SEQ ID NO: 151 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcaaaaaaggggaattaact

SEQ ID NO: 152 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaagttccatctcaaaaggg

SEQ ID NO: 153 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttatgaaagttccatctca

SEQ ID NO: 154 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tactgccaagaaactttatg

SEQ ID NO: 155 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttatttgaagcaaaataaac

SEQ ID NO: 156 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ataagtttatttgaagcaaa

SEQ ID NO: 157 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agacaacttttcaaataagt

SEQ ID NO: 158 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgatgaatccatttgacttg

SEQ ID NO: 159 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacaggtgatgaatccattt

SEQ ID NO: 160 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aggtgtcaatgcatgacagg

SEQ ID NO: 161 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
accaattaagtctgggtatc

SEQ ID NO: 162 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tggccaatgcaagaacaaat

SEQ ID NO: 163 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaaaaaaaaattttcactt

SEQ ID NO: 164 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcaaaactagatttcaaaag

SEQ ID NO: 165 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtgcggtcacccagacttat

SEQ ID NO: 166 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttaggtgcggtcacccagac

SEQ ID NO: 167 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggtactgcttaccattttag

SEQ ID NO: 168 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcactaagaaaaagccggag

SEQ ID NO: 169 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catcacccaaatgcacagag

SEQ ID NO: 170 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacaggccatgtaaatagaa

SEQ ID NO: 171 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacttcccaatggagattta

SEQ ID NO: 172 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agaatctttttagaaggcat

SEQ ID NO: 173 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttgcccactcccccaaata

SEQ ID NO: 174 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cattagaaaataatcaacat

SEQ ID NO: 175 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgatatgctttgctacaaag

SEQ ID NO: 176 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctgatattcccttttcaat

SEQ ID NO: 177 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaatcccaaactaggaaggt

SEQ ID NO: 178 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgcaattaattccacttttc

SEQ ID NO: 179 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcttctactttatccctac

SEQ ID NO: 180 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggcacaagataatttgtggt

SEQ ID NO: 181 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aggcctcttaatggatttca

SEQ ID NO: 182 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccctaattcttaaagctatc

SEQ ID NO: 183 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acttccagacagacaaccca

SEQ ID NO: 184 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaagcccattccacttaac

SEQ ID NO: 185 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcccccacctcctggagga

SEQ ID NO: 186 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgtattcaatgttaccaca

SEQ ID NO: 187 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgtaagcgattttattcaa

SEQ ID NO: 188 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcattgtgagagtgtgagtt

SEQ ID NO: 189 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcttttacatacttaacaat

SEQ ID NO: 190 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aacagagaatcaatgttatt

SEQ ID NO: 191 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aattagttacaaaaaagtac

SEQ ID NO: 192 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aatgagctcaactctcacag

SEQ ID NO: 193 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcacattcttccaactagaa

SEQ ID NO: 194 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aactaccaacacaacaaata

SEQ ID NO: 195 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttaggtaagggcattaggta

SEQ ID NO: 196 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acctatttatcataatctaa

SEQ ID NO: 197 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgtaacttgcaaaatgacaa

SEQ ID NO: 198 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acttcattgataaatgttta

SEQ ID NO: 199 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gattacaagtctaaaggatg

SEQ ID NO: 200 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaataatgaaacaatgtggc

SEQ ID NO: 201 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atccctttacagaggaaact

SEQ ID NO: 202 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaaattaaaacaactcaag

SEQ ID NO: 203 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cagattcagatgcagaaaaa

SEQ ID NO: 204 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tacagggtttggaaatcatg

SEQ ID NO: 205 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaatgcaaaattcagatgg

SEQ ID NO: 206 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgagtaatagtgcaagtgct

SEQ ID NO: 207 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgttaccatgttactgctgc

SEQ ID NO: 208 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tccccgagtaccattttaag

SEQ ID NO: 209 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtcagtttagtctttggagg

SEQ ID NO: 210 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctgggctccttgaaggctt

SEQ ID NO: 211 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgttgacaagttaacttacc

SEQ ID NO: 212 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagaagggattaaaccatgc

SEQ ID NO: 213 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgaaatttacacaagtgta

SEQ ID NO: 214 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagccttctatgaccagtaa

SEQ ID NO: 215 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaaggccactcaacattga

SEQ ID NO: 216 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agtaccataaacatgttaat

SEQ ID NO: 217 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ataaatacccgtatctatgc

SEQ ID NO: 218 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaaatcttcttagggtaaa

SEQ ID NO: 219 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtttaagtacttttaaactt

SEQ ID NO: 220 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaacaaatctttgccaaata

SEQ ID NO: 221 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgaccaaatagatttttaa

SEQ ID NO: 222 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttagaatgaatgcatttaga

SEQ ID NO: 223 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatctggttcaaaaaatttt

SEQ ID NO: 224 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgtcaaaaaaaaattttatt

SEQ ID NO: 225 - DNA, 22 bp, synthetic construct
  misc_feature 1..22: note = Synthetic
  source 1..22: mol_type = other DNA
gggaaatctggtgtatgttgtc

SEQ ID NO: 226 - DNA, 20 bp, synthetic construct
  misc_feature 1..20: note = Synthetic
  source 1..20: mol_type = other DNA
tttcaggtcctgttcggttg

SEQ ID NO: 227 - DNA, 20 bp, synthetic construct
  misc_feature 1..20: note = Synthetic
  source 1..20: mol_type = other DNA
gcgctgtacagaggacatga

SEQ ID NO: 228 - DNA, 20 bp, synthetic construct
  misc_feature 1..20: note = Synthetic
  source 1..20: mol_type = other DNA
agttcatcccaccacccata

SEQ ID NO: 229 - DNA, 22 bp, synthetic construct
  misc_feature 1..22: note = Synthetic
  source 1..22: mol_type = other DNA
gaagatcaagatcattgctcct

SEQ ID NO: 230 - DNA, 20 bp, synthetic construct
  misc_feature 1..20: note = Synthetic
  source 1..20: mol_type = other DNA
tactcctgcttgctgatcca

SEQ ID NO: 231 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcatctgtctcatccatttt

SEQ ID NO: 232 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agcatctgtctcatccattt

SEQ ID NO: 233 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagcatctgtctcatccatt

SEQ ID NO: 234 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaagcatctgtctcatccat

SEQ ID NO: 235 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttttcactttcactgctga

SEQ ID NO: 236 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctggactgctcttttcactt

SEQ ID NO: 237 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gttttctggactgctctttt

SEQ ID NO: 238 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gatgttttctggactgctct

SEQ ID NO: 239 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gagacccaacactattaaat

SEQ ID NO: 240 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttttccatgggagacccaac

SEQ ID NO: 241 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggttgttttccatgggagac

SEQ ID NO: 242 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttaagatctttcttgacctg

SEQ ID NO: 243 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agtcttaagatctttcttga

SEQ ID NO: 244 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgaccagtcttaagatcttt

SEQ ID NO: 245 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgaatgaccagtcttaaga

SEQ ID NO: 246 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttgaatgaccagtcttaag

SEQ ID NO: 247 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctttgaatgaccagtcttaa

SEQ ID NO: 248 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctttgaatgaccagtctta

SEQ ID NO: 249 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccctttgaatgaccagtctt

SEQ ID NO: 250 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acccctttgaatgaccagtc

SEQ ID NO: 251 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aacccctttgaatgaccagt

SEQ ID NO: 252 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaacccctttgaatgaccag

SEQ ID NO: 253 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaacccctttgaatgacca

SEQ ID NO: 254 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccaaacccctttgaatgacc

SEQ ID NO: 255 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gccaaacccctttgaatgac

SEQ ID NO: 256 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agccaaacccctttgaatga

SEQ ID NO: 257 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagccaaacccctttgaatg

SEQ ID NO: 258 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaagccaaacccctttgaa

SEQ ID NO: 259 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcatattccgtaaaacgaa

SEQ ID NO: 260 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgtgtttcatattccgtaaa

SEQ ID NO: 261 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tactttcacttgtgtttcat

SEQ ID NO: 262 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacattactttcacttgtgt

SEQ ID NO: 263 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgctgtgacattactttcac

SEQ ID NO: 264 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atcatatgtcgctgtgacat

SEQ ID NO: 265 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catctatcatatgtcgctgt

SEQ ID NO: 266 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctttgcttagaattaggaag

SEQ ID NO: 267 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcttctcaaaggctcatctt

SEQ ID NO: 268 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttctgcttctcaaaggctc

SEQ ID NO: 269 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acacttttctgcttctcaaa

SEQ ID NO: 270 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtcatgtcctctgtacagcg

SEQ ID NO: 271 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctcagtcatgtcctctgta

SEQ ID NO: 272 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgcagctcatcctcagtcat

SEQ ID NO: 273 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
actcccgcagctcatcctca

SEQ ID NO: 274 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcctctccacaaagagactg

SEQ ID NO: 275 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgtgcttaggttcggcattg

SEQ ID NO: 276 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgtgcttaggttcggcatt

SEQ ID NO: 277 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attgtgcttaggttcggcat

SEQ ID NO: 278 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tattgtgcttaggttcggca

SEQ ID NO: 279 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctattgtgcttaggttcggc

SEQ ID NO: 280 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgctattgtgcttaggttcg

SEQ ID NO: 281 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgctattgtgcttaggttc

SEQ ID NO: 282 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attgctattgtgcttaggtt

SEQ ID NO: 283 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tattgctattgtgcttaggt

SEQ ID NO: 284 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctattgctattgtgcttagg

SEQ ID NO: 285 - DNA, 20 bp, synthetic construct
  misc_feature 1..20: note = Synthetic
  source 1..20: mol_type = other DNA
actgtctattgctattgtgc

SEQ ID NO: 286 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaatcttccacttctttcta

SEQ ID NO: 287 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcccaaagccacctggatta

SEQ ID NO: 288 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaatccaccctgattccca

SEQ ID NO: 289 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
actgaacaaaccaattttga

SEQ ID NO: 290 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tccacactgaacaaaccaat

SEQ ID NO: 291 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tactccacactgaacaaacc

SEQ ID NO: 292 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgctgaatatactccacact

SEQ ID NO: 293 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaaatactgctgaatatac

SEQ ID NO: 294 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttttctaaagaaaaatgtc

SEQ ID NO: 295 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttcctttttctaaagaaaa

SEQ ID NO: 296 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcctttagctcttccttttt

SEQ ID NO: 297 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaattcctttagctcttcc

SEQ ID NO: 298 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttataaaattcctttagct

SEQ ID NO: 299 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcaatatttcaacctttca

SEQ ID NO: 300 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaccactcaatatttcaacc

SEQ ID NO: 301 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctttcaaccactcaatattt

SEQ ID NO: 302 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cagcagttcactttcaacca

SEQ ID NO: 303 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcaaacagcagttcactttc

SEQ ID NO: 304 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgacaagtccagggataaa

SEQ ID NO: 305 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttgacaagtccagggataa

SEQ ID NO: 306 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acttgacaagtccagggata

SEQ ID NO: 307 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacttgacaagtccagggat

SEQ ID NO: 308 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcacttgacaagtccaggga

SEQ ID NO: 309 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcacttgacaagtccaggg

SEQ ID NO: 310 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attcacttgacaagtccagg

SEQ ID NO: 311 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aattcacttgacaagtccag

SEQ ID NO: 312 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaattcacttgacaagtcca

SEQ ID NO: 313 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aagaattcacttgacaagtc

SEQ ID NO: 314 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgaacatgcaaagaattcac

SEQ ID NO: 315 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgttttgaacatgcaaagaa

SEQ ID NO: 316 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtttccgttttgaacatgca

SEQ ID NO: 317 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catgatctcctaaaaggaga

SEQ ID NO: 318 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacaccatgatctcctaaaa

SEQ ID NO: 319 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaacactgtgacaccatga

SEQ ID NO: 320 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agaaccaaacactgtgacac

SEQ ID NO: 321 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gaattcttgaatgagaaagc

SEQ ID NO: 322 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgacgaattcttgaatgag

SEQ ID NO: 323 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtgatgcgtgatgacgaatt

SEQ ID NO: 324 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggcctgtgatgcgtgatgac

SEQ ID NO: 325 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgatacgactccatgaaata

SEQ ID NO: 326 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgatacgactccatgaaat

SEQ ID NO: 327 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gttgatacgactccatgaaa

SEQ ID NO: 328 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgttgatacgactccatgaa

SEQ ID NO: 329 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tagcgttgatacgactccat

SEQ ID NO: 330 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atagcgttgatacgactcca

SEQ ID NO: 331 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcatagcgttgatacgactc

SEQ ID NO: 332 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcatagcgttgatacgact

SEQ ID NO: 333 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgttcatagcgttgatacga

SEQ ID NO: 334 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccttgcgttcatagcgttga

SEQ ID NO: 335 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gccttgcgttcatagcgttg

SEQ ID NO: 336 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agccttgcgttcatagcgtt

SEQ ID NO: 337 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acagccttgcgttcatagcg

SEQ ID NO: 338 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacagccttgcgttcatagc

SEQ ID NO: 339 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcacagccttgcgttcatag

SEQ ID NO: 340 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atcacagccttgcgttcata

SEQ ID NO: 341 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatcacagccttgcgttcat

SEQ ID NO: 342 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catatcacagccttgcgttc

SEQ ID NO: 343 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tccatatcacagccttgcgt

SEQ ID NO: 344 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gttccatatcacagccttgc

SEQ ID NO: 345 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggttccatatcacagccttg

SEQ ID NO: 346 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tggttccatatcacagcctt

SEQ ID NO: 347 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccttctggttccatatcaca

SEQ ID NO: 348 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gccttctggttccatatcac

SEQ ID NO: 349 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacagccttctggttccata

SEQ ID NO: 350 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caaaagttcagacagccttc

SEQ ID NO: 351 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggtttcaaaagttcagacag

SEQ ID NO: 352 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccaccatcaatcccacacaa

SEQ ID NO: 353 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caccaccatcaatcccacac

SEQ ID NO: 354 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcaccaccatcaatcccaca

SEQ ID NO: 355 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ggcaccaccatcaatcccac

SEQ ID NO: 356 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cggcaccaccatcaatccca

SEQ ID NO: 357 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcggcaccaccatcaatccc

SEQ ID NO: 358 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcggcaccaccatcaatcc

SEQ ID NO: 359 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctcggcaccaccatcaatc

SEQ ID NO: 360 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcctcggcaccaccatcaat

SEQ ID NO: 361 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atgcctcggcaccaccatca

SEQ ID NO: 362 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catgcctcggcaccaccatc

SEQ ID NO: 363 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tcatgcctcggcaccaccat

SEQ ID NO: 364 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttcatgcctcggcaccacca

SEQ ID NO: 365 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttcatgcctcggcaccacc

SEQ ID NO: 366 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctttcatgcctcggcaccac

SEQ ID NO: 367 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cctttcatgcctcggcacca

SEQ ID NO: 368 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcctttcatgcctcggcacc

SEQ ID NO: 369 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agcctttcatgcctcggcac

SEQ ID NO: 370 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tagcctttcatgcctcggca

SEQ ID NO: 371 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctagcctttcatgcctcggc

SEQ ID NO: 372 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tactagcctttcatgcctcg

SEQ ID NO: 373 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atactagcctttcatgcctc

SEQ ID NO: 374 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcatactagcctttcatgc

SEQ ID NO: 375 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctcatactagcctttcatg

SEQ ID NO: 376 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgctcatactagcctttcat

SEQ ID NO: 377 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctcgctcatactagcctttc

SEQ ID NO: 378 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cttttctcgctcatactagc

SEQ ID NO: 379 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccttttctcgctcatactag

SEQ ID NO: 380 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tctccttttctcgctcatac

SEQ ID NO: 381 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cgctctctccttttctcgct

SEQ ID NO: 382 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcgctctctccttttctcgc

SEQ ID NO: 383 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acgcgctctctccttttctc

SEQ ID NO: 384 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tctctgcacgcgctctctcc

SEQ ID NO: 385 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caagtctctgcacgcgctct

SEQ ID NO: 386 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
accaagtctctgcacgcgct

SEQ ID NO: 387 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccaccaagtctctgcacgcg

SEQ ID NO: 388 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aaaatatccattatgcacca

SEQ ID NO: 389 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
caagttaaaaaatatccatt

SEQ ID NO: 390 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gacacatctcgccaagttaa

SEQ ID NO: 391 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
accaaagccacaggattgag

SEQ ID NO: 392 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctgcacactctctcaccaaa

SEQ ID NO: 393 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctctgcacactctctcacca

SEQ ID NO: 394 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tctctgcacactctctcacc

SEQ ID NO: 395 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctctctgcacactctctcac

SEQ ID NO: 396 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctctctgcacactctctca

SEQ ID NO: 397 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgctctctgcacactctctc

SEQ ID NO: 398 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgctctctgcacactctct

SEQ ID NO: 399 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attgctctctgcacactctc

SEQ ID NO: 400 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tatcattgctctctgcacac

SEQ ID NO: 401 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ctatcattgctctctgcaca

SEQ ID NO: 402 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gctatcattgctctctgcac

SEQ ID NO: 403 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgctatcattgctctctgc

SEQ ID NO: 404 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tttgctatcattgctctctg

SEQ ID NO: 405 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
atttgctatcattgctctct

SEQ ID NO: 406 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttatttgctatcattgctct

SEQ ID NO: 407 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtacattatttgctatcatt

SEQ ID NO: 408 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cattcgtacattatttgcta

SEQ ID NO: 409 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
catatcagtaacagaagaga

SEQ ID NO: 410 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acttacatatcagtaacaga

SEQ ID NO: 411 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacttacatatcagtaacag

SEQ ID NO: 412 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acacttacatatcagtaaca

SEQ ID NO: 413 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cacacttacatatcagtaac

SEQ ID NO: 414 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ccacacttacatatcagtaa

SEQ ID NO: 415 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
tgccacacttacatatcagt

SEQ ID NO: 416 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttgccacacttacatatcag

SEQ ID NO: 417 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
attgccacacttacatatca

SEQ ID NO: 418 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
acattgccacacttacatat

SEQ ID NO: 419 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
agttcacattgccacactta

SEQ ID NO: 420 - DNA, 21 bp, synthetic construct
  misc_feature 1..21: note = Synthetic
  source 1..21: mol_type = other DNA
gagaacatggactgagcttgt

SEQ ID NO: 421 - DNA, 22 bp, synthetic construct
  misc_feature 1..22: note = Synthetic
  source 1..22: mol_type = other DNA
cagacattagaacttccaccct

SEQ ID NO: 422 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
cccctttgaatgaccagtct

SEQ ID NO: 423 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
gctattgtgcttaggttcgg

SEQ ID NO: 424 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
tcacttgacaagtccaggga

SEQ ID NO: 425 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
ttcatgcctcggcaccacca

SEQ ID NO: 426 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
tcgctcatactagcctttca

SEQ ID NO: 427 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
accaccaagtctctgcacgc

SEQ ID NO: 428 - DNA, 20 bp, synthetic construct
  misc_feature 1..20: note = Synthetic construct
  source 1..20: mol_type = other DNA
  misc_feature 1..5: note = RNA
  misc_feature 6: note = DNA
  misc_feature 7: note = RNA
  misc_feature 8..15: note = DNA
  misc_feature 16..20: note = RNA
gctatcattgctctctgcac

SEQ ID NO: 429 - DNA, 20 bp, synthetic construct
  misc_feature 1..20: note = Synthetic
  source 1..20: mol_type = other DNA
acccctttgaatgaccagtc

SEQ ID NO: 430 - DNA, 20 bp, synthetic construct
  misc_feature 1..20: note = Synthetic
  source 1..20: mol_type = other DNA
aacccctttgaatgaccagt

SEQ ID NO: 431 - DNA, 20 bp, synthetic construct
  misc_feature 1..20: note = Synthetic
  source 1..20: mol_type = other DNA
ccaaacccctttgaatgacc

SEQ ID NO: 432 - DNA, 20 bp, synthetic construct
  misc_feature 1..20: note = Synthetic
  source 1..20: mol_type = other DNA
gttcactttcaaccactcaa

SEQ ID NO: 433 - DNA, 20 bp, synthetic construct
  misc_feature 1..20: note = Synthetic
  source 1..20: mol_type = other DNA
cacttgacaagtccagggat

SEQ ID NO: 434 - DNA, 20 bp, synthetic construct
  misc_feature 1..20: note = Synthetic
  source 1..20: mol_type = other DNA
tcacttgacaagtccaggga

SEQ ID NO: 435 - DNA, 20 bp, synthetic construct
  misc_feature 1..20: note = Synthetic
  source 1..20: mol_type = other DNA
cggcaccaccatcaatccca

SEQ ID NO: 436 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
cagtcttaagatctttcttg

SEQ ID NO: 437 - DNA, 20 bp, synthetic construct
  misc_feature 1..20: note = Synthetic
  source 1..20: mol_type = other DNA
caaaggctcatcttggcttt

SEQ ID NO: 438 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtgcttaggttaggcattgg

SEQ ID NO: 439 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
aatatccattatgcaccacc

SEQ ID NO: 440 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gtgcttaggttcggcattgg

SEQ ID NO: 441 - DNA, 20 bp, synthetic construct
  misc_feature 1..20: note = Synthetic
  source 1..20: mol_type = other DNA
aggttcggcattggatatat

SEQ ID NO: 442 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
ttaggttcggcattggatat

SEQ ID NO: 443 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
gcttaggttcggcattggat

SEQ ID NO: 444 - DNA, 20 bp, synthetic construct
  misc_feature 1..20: note = Synthetic
  source 1..20: mol_type = other DNA
ctgcacgcgctctctccttt

SEQ ID NO: 445 - RNA, 20 nt, synthetic construct
  misc_feature 1..20: note = Synthetic
  source 1..20: mol_type = other RNA
ctctgcacgcgctctctcct

SEQ ID NO: 446 - RNA, 20 nt, synthetic construct
  misc_feature 1..20: note = Synthetic
  source 1..20: mol_type = other RNA
gtctctgcacgcgctctctc

SEQ ID NO: 447 - RNA, 20 nt, synthetic construct
  misc_feature 1..20: note = Synthetic
  source 1..20: mol_type = other RNA
aagtctctgcacgcgctctc

SEQ ID NO: 448 - RNA, 20 nt, synthetic construct
  misc_feature 1..20: note = Synthetic
  source 1..20: mol_type = other RNA
ccaagtctctgcacgcgctc

SEQ ID NO: 449 - RNA, 20 nt, synthetic construct
  misc_feature 1..20: note = Synthetic
  source 1..20: mol_type = other RNA
caccaagtctctgcacgcgc

SEQ ID NO: 450 - RNA, 20 nt, synthetic construct
  misc_feature 1..20: note = Synthetic
  source 1..20: mol_type = other RNA
accaccaagtctctgcacgc

SEQ ID NO: 451 - RNA, 20 nt, synthetic construct
  misc_feature 1..20: note = Synthetic
  source 1..20: mol_type = other RNA
gcaccaccaagtctctgcac

SEQ ID NO: 452 - RNA, 20 nt, synthetic construct
  misc_feature 1..20: note = Synthetic
  source 1..20: mol_type = other RNA
atgcaccaccaagtctctgc

SEQ ID NO: 453 - RNA, 20 nt, synthetic construct
  misc_feature 1..20: note = Synthetic
  source 1..20: mol_type = other RNA
ttatgcaccaccaagtctct

SEQ ID NO: 454 - RNA, 20 nt, synthetic construct
  misc_feature 1..20: note = Synthetic
  source 1..20: mol_type = other RNA
cattatgcaccaccaagtct

SEQ ID NO: 455 - RNA, 20 nt, synthetic construct
  misc_feature 1..20: note = Synthetic
  source 1..20: mol_type = other RNA
tccattatgcaccaccaagt

SEQ ID NO: 456 - RNA, 20 nt, synthetic construct
  misc_feature 1..20: note = Synthetic
  source 1..20: mol_type = other RNA
gcaaagcacaccacaagctc

SEQ ID NO: 457 - RNA, 20 nt, synthetic construct
  misc_feature 1..20: note = Synthetic
  source 1..20: mol_type = other RNA
ctgcaaagcacaccacaagc

SEQ ID NO: 458 - RNA, 20 nt, synthetic construct
  misc_feature 1..20: note = Synthetic
  source 1..20: mol_type = other RNA
tcctgcaaagcacaccacaa

SEQ ID NO: 459 - RNA, 20 nt, synthetic construct
  misc_feature 1..20: note = Synthetic
  source 1..20: mol_type = other RNA
cctcctgcaaagcacaccac

SEQ ID NO: 460 - RNA, 20 nt, synthetic construct
  misc_feature 1..20: note = Synthetic
  source 1..20: mol_type = other RNA
gtcctcctgcaaagcacacc

SEQ ID NO: 461 - RNA, 20 nt, synthetic construct
  misc_feature 1..20: note = Synthetic
  source 1..20: mol_type = other RNA
aagtcctcctgcaaagcaca

SEQ ID NO: 462 - RNA, 20 nt, synthetic construct
  misc_feature 1..20: note = Synthetic
  source 1..20: mol_type = other RNA
tcaagtcctcctgcaaagca

SEQ ID NO: 463 - RNA, 20 nt, synthetic construct
  misc_feature 1..20: note = Synthetic
  source 1..20: mol_type = other RNA
cttcaagtcctcctgcaaag

SEQ ID NO: 464 - RNA, 20 nt, synthetic construct
  misc_feature 1..20: note = Synthetic
  source 1..20: mol_type = other RNA
caagtcctcctgcaaagcac

SEQ ID NO: 465 - RNA, 20 nt, synthetic construct
  misc_feature 1..20: note = Synthetic
  source 1..20: mol_type = other RNA
ttcaagtcctcctgcaaagc

SEQ ID NO: 466 - RNA, 20 nt, synthetic construct
  misc_feature 1..20: note = Synthetic
  source 1..20: mol_type = other RNA
cagttttatttaagaatttc

SEQ ID NO: 467 - RNA, 22 nt, synthetic construct
  misc_feature 1..22: note = Synthetic
  source 1..22: mol_type = other RNA
gggcatgcagaattccttctac

SEQ ID NO: 468 - RNA, 20 nt, synthetic construct
  misc_feature 1..20: note = Synthetic
  source 1..20: mol_type = other RNA
ccatcgtcttccgatggtat

SEQ ID NO: 469 - RNA, 19 nt, synthetic construct
  misc_feature 1..19: note = Synthetic
  source 1..19: mol_type = other RNA
gctcaagcatggattctaa

SEQ ID NO: 470 - RNA, 19 nt, synthetic construct
  misc_feature 1..19: note = Synthetic
  source 1..19: mol_type = other RNA
caatcaaggtagtaatatg

SEQ ID NO: 471 - RNA, 19 nt, synthetic construct
  misc_feature 1..19: note = Synthetic
  source 1..19: mol_type = other RNA
gggcttcgctacaggaatc

SEQ ID NO: 472 - RNA, 19 nt, synthetic construct
  misc_feature 1..19: note = Synthetic
  source 1..19: mol_type = other RNA
cagggtggatttggtaata

SEQ ID NO: 473 - DNA, 17 bp, synthetic construct
  misc_feature 1..17: note = Synthetic
  source 1..17: mol_type = other DNA
tccacactgaacaaacc

SEQ ID NO: 474 - RNA, 21 nt, synthetic construct
  misc_feature 1..21: note = Synthetic
  source 1..21: mol_type = other RNA
aaggaaaatggatgagacaga

SEQ ID NO: 475 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
gcccctggaaactgggctgt

SEQ ID NO: 476 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
cactttcactgctgatgaag

SEQ ID NO: 477 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
actattaaatcggatgtttt

SEQ ID NO: 478 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ccaacactattaaatcggat

SEQ ID NO: 479 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
acctggattaccaccaaatc

SEQ ID NO: 480 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
cacctggattaccaccaaat

SEQ ID NO: 481 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ccacctggattaccaccaaa

SEQ ID NO: 482 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
gccacctggattaccaccaa

SEQ ID NO: 483 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
aagccacctggattaccacc

SEQ ID NO: 484 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
cataccccaactgctctgta

SEQ ID NO: 485 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
tggctggctaacatgcccat

SEQ ID NO: 486 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
cctgactggttctgctggct

SEQ ID NO: 487 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
atgggcctgactggttctgc

SEQ ID NO: 488 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
acccgatgggcctgactggt

SEQ ID NO: 489 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
tttggttattacccgatggg

SEQ ID NO: 490 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ttggttttggttattacccg

SEQ ID NO: 491 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ttgccttggttttggttatt

SEQ ID NO: 492 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ccattaaaaccactgcccga

SEQ ID NO: 493 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
tgcttgagccaaagcctcca

SEQ ID NO: 494 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ttagaatccatgcttgagcc

SEQ ID NO: 495 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
gaagacttagaatccatgct

SEQ ID NO: 496 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
agccagaagacttagaatcc

SEQ ID NO: 497 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ccaaccaaccacaaccccac

SEQ ID NO: 498 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ctataccaaccaaccacaac

SEQ ID NO: 499 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
tcccaccattctataccaac

SEQ ID NO: 500 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
tgaattcccaccattctata

SEQ ID NO: 501 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
aagtctctgcacgcgctctc

SEQ ID NO: 502 - DNA, 20 bp, synthetic construct
  misc_feature 1..20: note = Synthetic construct
  source 1..20: mol_type = other DNA
  misc_feature 1..5: note = RNA
  misc_feature 6..15: note = DNA
  misc_feature 16..20: note = RNA
ccaagtctctgcacgcgctc

(19) United States
(12) Patent Application Publication
(10) Pub. No.: US 2026/0085310 A1
(43) Pub. Date: Mar. 26, 2026

(54) ANTISENSE OLIGONUCLEOTIDE TARGETING TDP-43 MRNA OR PRE-MRNA

(71) Applicant: NIPPON SHINYAKU CO., LTD., Kyoto-shi, Kyoto (JP)

(72) Inventors: Akihiro MURATA, Ibaraki (JP); Mikiya FUJIWARA, Ibaraki (JP)

(73) Assignee: NIPPON SHINYAKU CO., LTD., Kyoto-shi, Kyoto (JP)

(21) Appl. No.: 18/867,212

(22) PCT Filed: May 26, 2023
(86) PCT No.: PCT/JP2023/019791

§ 371 Date: Aug. 22, 2025

(30) Foreign Application Priority Data
May 27, 2022 (JP) 2022-087196
May 12, 2023 (JP) 2023-079365

(51) Int. Cl. C12N 15/113; A61K 31/7088; A61K 48/00; A61P 25/28

(52) U.S. Cl. CPC C12N 15/113; A61K 31/7088; A61K 48/005; A61P 25/28; C12N 2310/11; C12N 2310/341

(57) ABSTRACT

In the present specification, an antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1 is provided.

### TECHNICAL FIELD

The present invention relates to an antisense oligonucleotide targeting TDP-43 mRNA or pre-mRNA, or a pharmaceutically acceptable salt thereof, or a hydrate thereof, and a pharmaceutical composition or the like comprising the antisense oligonucleotide or a pharmaceutically acceptable salt thereof, or a hydrate thereof.

### BACKGROUND ART

TAR DNA-binding protein 43 (TDP-43) is a highly conserved, and ubiquitously expressed RNA/DNA binding protein belonging to the heterogeneous nuclear ribonucleoprotein (hnRNP) family (Non Patent Literature 1). It is known that accumulation of TDP-43 relates to a large number of neurodegenerative diseases (designated as “TDP-43 proteinopathies”) including amyotrophic lateral sclerosis (ALS). For TDP-43 proteinopathies, antisense oligonucleotides for adjusting the expression level of TDP-43 (Patent Literatures 1 and 2, and Non Patent Literature 2), and antisense oligonucleotides for increasing alternative splicing of intron 6 of TDP-43 mRNA (Patent Literature 3) or the like have been studied, but there is no effective treatment.

### CITATION LIST

### Patent Literature

Patent Literature 1: International Publication No. WO2019/013141
Patent Literature 2: International Publication No. WO2022/120410
Patent Literature 3: International Publication No. WO2022/113799

### Non Patent Literature

Non Patent Literature 1: de Boer E M J et al., J. Neurol Neurosurg Psychiatry, 2021, Vol. 92 (1), pp. 86-95
Non Patent Literature 2: T Takeuchi et al., Molecular Therapy Nucleic Acids, 2023, Vol. 31, pp. 353-366

### SUMMARY OF INVENTION

Under these circumstances, it is desired to provide a novel TDP-43 proteinopathy therapeutic agent and the like.

The present invention provides an antisense oligonucleotide targeting an TDP-43 mRNA or pre-mRNA, or a pharmaceutically acceptable salt thereof, or a hydrate thereof, and a pharmaceutical composition or the like comprising the antisense oligonucleotide or a pharmaceutically acceptable salt thereof, or a hydrate thereof as follows.

(1-1) An antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1.

(1-2) The antisense oligonucleotide or a pharmaceutically acceptable salt thereof, or a hydrate thereof, consisting of 15 to 30 nucleotides complementary to a nucleic acid comprising at least 15 consecutive bases in a target region selected from the group consisting of positions 1 to 102, 159 to 842, 879 to 1822, and 1874 to 4182 from the 5′ end of the base sequence of SEQ ID NO: 1.

(1-3) An antisense oligonucleotide or a pharmaceutically acceptable salt thereof, or a hydrate thereof, consisting of 15 to 25 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1.

(1-4) The antisense oligonucleotide or the pharmaceutically acceptable salt thereof, or the hydrate thereof according to any one of (1-1) to (1-3), complementary to a nucleic acid comprising at least 15 consecutive bases in a target region selected from the group consisting of positions 1 to 100, 161 to 840, 881 to 1820, and 1876 to 4180 from the 5′ end of the base sequence of SEQ ID NO: 1. (In (1-1) to (1-4), an antisense oligonucleotide or a pharmaceutically acceptable salt thereof, or a hydrate thereof having a sequence consisting of a base sequence selected from the group consisting of SEQ ID NOs: 65, 436 to 440, 444 to 449, 451 to 452, and 454 to 474 is preferably excluded,
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is preferably excluded, and/or
an antisense oligonucleotide having a sequence consisting of a base sequence of SEQ ID NO: 453 or a pharmaceutically acceptable salt thereof, or a hydrate thereof, in which positions 1 to 2, 4, 6, 8 to 9, 11 to 12, and 16 to 20 from the 5′ end of the base sequence of SEQ ID NO: 453 are ENA: 2′-O,4′-C-ethylene bridged nucleic acid, and positions 3, 5, 7, 10, and 13 to 15 are ribonucleotides comprising a 2′-OMe group, is preferably excluded.)

(2) The antisense oligonucleotide or a pharmaceutically acceptable salt thereof, or a hydrate thereof according to any one of (1-1) to (1-4), comprising:
(i) a base sequence selected from the group consisting of SEQ ID NOs: 2 to 64, 66 to 224, 422 to 423, 426 to 427, and 432;
(ii) a base sequence having addition, deletion, or substitution of one or several bases in a base sequence selected from the group consisting of SEQ ID NOs: 2 to 64, 66 to 224, 422 to 423, 426 to 427, and 432; or
(iii) a base sequence having 90% or more sequence identity with a base sequence selected from the group consisting of SEQ ID NOs: 2 to 64, 66 to 224, 422 to 423, 426 to 427, and 432.

(3-1) The antisense oligonucleotide or the pharmaceutically acceptable salt thereof, or the hydrate thereof according to any one of (1-1) to (1-3) complementary to a nucleic acid comprising at least 15 consecutive bases in a target region selected from the group consisting of positions 1 to 102, 159 to 622, 639 to 842, 879 to 1442, 1459 to 1497, 1499 to 1522, 1539 to 1702, 1719 to 1762, 1779 to 1802, 1874 to 1937, 1939 to 2302, 2319 to 3162, 3199 to 3242, 3279 to 3382, 3399 to 3482, 3539 to 3562, 3579 to 3602, 3619 to 3662, 3679 to 3702, and 3759 to 4182 from the 5′ end of the base sequence of SEQ ID NO: 1.

(3-2) The antisense oligonucleotide or the pharmaceutically acceptable salt thereof, or the hydrate thereof according to (3-1) complementary to a nucleic acid comprising at least 15 consecutive bases in a target region selected from the group consisting of positions 1 to 100, 161 to 620, 641 to 840, 881 to 1440, 1461 to 1495, 1501 to 1520, 1541 to 1700, 1721 to 1760, 1781 to 1800, 1876 to 1935, 1941 to 2300, 2321 to 3160, 3201 to 3240, 3281 to 3380, 3401 to 3480, 3541 to 3560, 3581 to 3600, 3621 to 3660, 3681 to 3700, and 3761 to 4180 from the 5′ end of the base sequence of SEQ ID NO: 1.

(4) The antisense oligonucleotide or the pharmaceutically acceptable salt thereof, or the hydrate thereof according to (3-1) or (3-2), comprising:
(i) a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224;
(ii) a base sequence having addition, deletion, or substitution of one or several bases in a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224; or
(iii) a base sequence having 90% or more sequence identity with a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224.

(5-1) The antisense oligonucleotide or the pharmaceutically acceptable salt thereof, or the hydrate thereof according to any one of (1-1) to (1-3) complementary to a nucleic acid comprising at least 15 consecutive bases in a target region selected from the group consisting of positions 19 to 42, 79 to 102, 159 to 182, 199 to 622, 639 to 662, 679 to 762, 779 to 802, 819 to 842, 879 to 1002, 1019 to 1402, 1419 to 1442, 1459 to 1497, 1499 to 1522, 1559 to 1642, 1659 to 1702, 1779 to 1802, 1874 to 1902, 1914 to 1937, 1959 to 2002, 2019 to 2302, 2319 to 2342, 2359 to 2802, 2819 to 3142, 3199 to 3222, 3299 to 3322, 3339 to 3362, 3399 to 3422, 3799 to 3842, 3879 to 3982, 4019 to 4042, and 4119 to 4142 from the 5′ end of the base sequence of SEQ ID NO: 1.

(5-2) The antisense oligonucleotide or the pharmaceutically acceptable salt thereof, or the hydrate thereof according to (5-1) complementary to a nucleic acid comprising at least 15 consecutive bases in a target region selected from the group consisting of positions 21 to 40, 81 to 100, 161 to 180, 201 to 620, 641 to 660, 681 to 760, 781 to 800, 821 to 840, 881 to 1000, 1021 to 1400, 1421 to 1440, 1461 to 1495, 1501 to 1520, 1561 to 1640, 1661 to 1700, 1781 to 1800, 1876 to 1900, 1916 to 1935, 1961 to 2000, 2021 to 2300, 2321 to 2340, 2361 to 2800, 2821 to 3140, 3201 to 3220, 3301 to 3320, 3341 to 3360, 3401 to 3420, 3801 to 3840, 3881 to 3980, 4021 to 4040, and 4121 to 4140 from the 5′ end of the base sequence of SEQ ID NO: 1.

(6) The antisense oligonucleotide or the pharmaceutically acceptable salt thereof, or the hydrate thereof according to (5-1) or (5-2), comprising:
(i) a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222;
(ii) a base sequence having addition, deletion, or substitution of one or several bases in a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222; or
(iii) a base sequence having 90% or more sequence identity with a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222.

(7-1) The antisense oligonucleotide or the pharmaceutically acceptable salt thereof, or the hydrate thereof according to any one of (1-1) to (1-3) complementary to a nucleic acid comprising at least 15 consecutive bases in a target region selected from the group consisting of positions 19 to 42, 159 to 182, 239 to 282, 319 to 342, 359 to 482, 499 to 602, 639 to 662, 679 to 762, 779 to 802, 819 to 842, 879 to 902, 919 to 1002, 1039 to 1342, 1359 to 1382, 1419 to 1442, 1459 to 1482, 1499 to 1522, 1559 to 1642, 1659 to 1702, 1779 to 1802, 1879 to 1902, 1914 to 1937, 1979 to 2002, 2019 to 2042, 2059 to 2302, 2319 to 2342, 2359 to 2422, 2446 to 2542, 2559 to 2622, 2639 to 2702, 2939 to 2962, 3019 to 3042, 3119 to 3142, and 3199 to 3222 from the 5′ end of the base sequence of SEQ ID NO: 1.

(7-2) The antisense oligonucleotide or the pharmaceutically acceptable salt thereof, or the hydrate thereof according to (7-1) complementary to a nucleic acid comprising at least 15 consecutive bases in a target region selected from the group consisting of positions 21 to 40, 161 to 180, 241 to 280, 321 to 340, 361 to 480, 501 to 600, 641 to 660, 681 to 760, 781 to 800, 821 to 840, 881 to 900, 921 to 1000, 1041 to 1340, 1361 to 1380, 1421 to 1440, 1461 to 1480, 1501 to 1520, 1561 to 1640, 1661 to 1700, 1781 to 1800, 1881 to 1900, 1916 to 1935, 1981 to 2000, 2021 to 2040, 2061 to 2300, 2321 to 2340, 2361 to 2420, 2448 to 2540, 2561 to 2620, 2641 to 2700, 2941 to 2960, 3021 to 3040, 3121 to 3140, and 3201 to 3220 from the 5′ end of the base sequence of SEQ ID NO: 1.

(8) The antisense oligonucleotide or the pharmaceutically acceptable salt thereof, or the hydrate thereof according to (7-1) or (7-2), comprising:
(i) a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176;
(ii) a base sequence having addition, deletion, or substitution of one or several bases in a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176; or
(iii) a base sequence having 90% or more sequence identity with a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176.

(9-1) The antisense oligonucleotide or the pharmaceutically acceptable salt thereof, or the hydrate thereof according to any one of (1-1) to (1-3) complementary to a nucleic acid comprising at least 15 consecutive bases in a target region selected from the group consisting of positions 239 to 262, 359 to 402, 419 to 462, 519 to 542, 559 to 602, 699 to 722, 819 to 842, 879 to 902, 919 to 982, 1091 to 1122, 1139 to 1162, 1179 to 1202, 1239 to 1262, 1279 to 1342, 1359 to 1382, 1459 to 1482, 1499 to 1522, 1559 to 1602, 1659 to 1682, 1779 to 1802, 1879 to 1902, 1979 to 2002, 2019 to 2042, 2059 to 2122, 2139 to 2216, 2219 to 2242, 2259 to 2302, 2446 to 2469, 2479 to 2502, and 2659 to 2682 from the 5′ end of the base sequence of SEQ ID NO: 1.

(9-2) The antisense oligonucleotide or the pharmaceutically acceptable salt thereof, or the hydrate thereof according to (9-1) complementary to a nucleic acid comprising at least 15 consecutive bases in a target region selected from the group consisting of positions 241 to 260, 361 to 400, 421 to 460, 521 to 540, 561 to 600, 701 to 720, 821 to 840, 881 to 900, 921 to 980, 1093 to 1120, 1141 to 1160, 1181 to 1200, 1241 to 1260, 1281 to 1340, 1361 to 1380, 1461 to 1480, 1501 to 1520, 1561 to 1600, 1661 to 1680, 1781 to 1800, 1881 to 1900, 1981 to 2000, 2021 to 2040, 2061 to 2120, 2141 to 2214, 2221 to 2240, 2261 to 2300, 2448 to 2467, 2481 to 2500, and 2661 to 2680 from the 5′ end of the base sequence of SEQ ID NO: 1.

(10) The antisense oligonucleotide or the pharmaceutically acceptable salt thereof, or the bydrate thereof according to (9-1) or (9-2), comprising:
(i) a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142;
(ii) a base sequence having addition, deletion, or substitution of one or several bases in a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142; or
(iii) a base sequence having 90% or more sequence identity with a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142.

(11) The antisense oligonucleotide or the pharmaceutically acceptable salt thereof, or the hydrate thereof according to any one of (2), (4), (6), (8), and (10), comprising the base sequence (i).

(12-1) An antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 194 to 217, 350 to 452, 500 to 612, 629 to 684, 689 to 732, 821 to 844, 874 to 972, 1089 to 1177, 1277 to 1338, 1349 to 1392, 1444 to 1537, 1544 to 1592, 1644 to 1697, 1769 to 1812, 1969 to 2012, 2051 to 2317, and 2469 to 2512 from the 5′ end of a base sequence of SEQ ID NO: 1.

(12-2) The antisense oligonucleotide or the pharmaceutically acceptable salt thereof, or the hydrate thereof according to (12-1), complementary to a nucleic acid comprising at least 15 consecutive bases in a target region selected from the group consisting of positions 196 to 215, 352 to 374, 376 to 413, 416 to 450, 502 to 610, 631 to 650, 653 to 682, 691 to 730, 823 to 842, 876 to 970, 1091 to 1175, 1279 to 1336, 1351 to 1390, 1446 to 1535, 1546 to 1590, 1646 to 1695, 1771 to 1810, 1971 to 2010, 2053 to 2250, 2252 to 2315, and 2471 to 2510 from the 5′ end of the base sequence of SEQ ID NO: 1. (In (12-1) to (12-2), an antisense oligonucleotide or a pharmaceutically acceptable salt thereof, or a hydrate thereof having a sequence consisting of a base sequence selected from the group consisting of SEQ ID NOs: 436 to 438, 440 to 449, 451 to 452, 454 to 455, and 472 to 474 is preferably excluded,
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is preferably excluded, and/or
an antisense oligonucleotide having a sequence consisting of a base sequence of SEQ ID NO: 453 or a pharmaceutically acceptable salt thereof, or a hydrate thereof, in which positions 1 to 2, 4, 6, 8 to 9, 11 to 12, and 16 to 20 from the 5′ end of the base sequence of SEQ ID NO: 453 are ENA: 2′-O,4′-C-ethylene bridged nucleic acid, and positions 3, 5, 7, 10, and 13 to 15 are ribonucleotides comprising a 2′-OMe group, is preferably excluded.)

(13) The antisense oligonucleotide or the pharmaceutically acceptable salt thereof, or the hydrate thereof according to (12-1) or (12-2), comprising:
(i) a base sequence selected from the group consisting of SEQ ID NOs: 231 to 419, 424 to 425, 428 to 431, 433 to 435, and 475 to 502;
(ii) a base sequence having addition, deletion, or substitution of one or several bases in a base sequence selected from the group consisting of SEQ ID NOs: 231 to 419, 424 to 425, 428 to 431, 433 to 435, and 475 to 502; or
(iii) a base sequence having 90% or more sequence identity with a base sequence selected from the group consisting of SEQ ID NOs: 231 to 419, 424 to 425, 428 to 431, 433 to 435, and 475 to 502.

(14-1) The antisense oligonucleotide or the pharmaceutically acceptable salt thereof, or the hydrate thereof according to (12-1) complementary to a nucleic acid comprising at least 15 consecutive bases in a target region selected from the group consisting of positions 194 to 217, 350 to 452, 500 to 612, 629 to 679, 689 to 732, 821 to 844, 874 to 912, 924 to 972, 1089 to 1177, 1277 to 1338, 1349 to 1392, 1449 to 1484, 1504 to 1532, 1544 to 1592, 1644 to 1697, 1769 to 1812, 1969 to 2012, 2051 to 2252, 2264 to 2317, and 2476 to 2512 from the 5′ end of the base sequence of SEQ ID NO: 1.

(14-2) The antisense oligonucleotide or the pharmaceutically acceptable salt thereof, or the hydrate thereof according to (14-1) complementary to a nucleic acid comprising at least 15 consecutive bases in a target region selected from the group consisting of positions 196 to 215, 352 to 450, 502 to 610, 631 to 650, 653 to 677, 691 to 730, 823 to 842, 876 to 910, 926 to 970, 1091 to 1175, 1279 to 1336, 1351 to 1390, 1451 to 1482, 1506 to 1530, 1546 to 1590, 1646 to 1695, 1771 to 1810, 1971 to 2010, 2053 to 2250, 2266 to 2315, and 2478 to 2510 from the 5′ end of the base sequence of SEQ ID NO: 1.

(15) The antisense oligonucleotide or the pharmaceutically acceptable salt thereof, or the hydrate thereof according to (14-1) or (14-2), comprising:
(i) a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502;
(ii) a base sequence having addition, deletion, or substitution of one or several bases in a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502; or
(iii) a base sequence having 90% or more sequence identity with a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502.

(16-1) The antisense oligonucleotide or the pharmaceutically acceptable salt thereof, or the hydrate thereof according to (12-1) complementary to a nucleic acid comprising at least 15 consecutive bases in a target region selected from the group consisting of positions 351 to 376, 384 to 452, 509 to 552, 569 to 607, 656 to 679, 821 to 844, 874 to 912, 927 to 952, 1109 to 1177, 1277 to 1338, 1364 to 1392, 1449 to 1484, 1504 to 1532, 1544 to 1587, 1646 to 1697, 1774 to 1812, 1984 to 2007, 2054 to 2127, 2131 to 2215, 2229 to 2252, 2264 to 2312, and 2478 to 2512 from the 5′ end of the base sequence of SEQ ID NO: 1.

(16-2) The antisense oligonucleotide or the pharmaceutically acceptable salt thereof, or the hydrate thereof according to (16-1) complementary to a nucleic acid comprising at least 15 consecutive bases in a target region selected from the group consisting of positions 353 to 374, 386 to 450, 511 to 550, 571 to 605, 658 to 677, 823 to 842, 876 to 910, 929 to 950, 1111 to 1175, 1279 to 1336, 1366 to 1390, 1451 to 1482, 1506 to 1530, 1546 to 1585, 1648 to 1695, 1776 to 1810, 1986 to 2005, 2056 to 2125, 2133 to 2213, 2231 to 2250, 2266 to 2310, and 2480 to 2510 from the 5′ end of the base sequence of SEQ ID NO: 1.

(17) The antisense oligonucleotide or the pharmaceutically acceptable salt thereof, or the hydrate thereof according to (16-1) or (16-2), comprising:
(i) a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502;
(ii) a base sequence having addition, deletion, or substitution of one or several bases in a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502; or
(iii) a base sequence having 90% or more sequence identity with a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502.

(18-1) The antisense oligonucleotide or the pharmaceutically acceptable salt thereof, or the hydrate thereof according to (12-1) complementary to a nucleic acid comprising at least 15 consecutive bases in a target region selected from the group consisting of positions 409 to 452, 509 to 548, 656 to 679, 874 to 907, 927 to 950, 1129 to 1172, 1294 to 1333, 1449 to 1484, 1504 to 1532, 1549 to 1587, 1646 to 1697, 1784 to 1807, 2057 to 2117, 2131 to 2215, 2266 to 2312, and 2478 to 2501 from the 5′ end of the base sequence of SEQ ID NO: 1.

(18-2) The antisense oligonucleotide or the pharmaceutically acceptable salt thereof, or the hydrate thereof according to (18-1) complementary to a nucleic acid comprising at least 15 consecutive bases in a target region selected from the group consisting of positions 411 to 450, 511 to 546, 658 to 677, 876 to 905, 929 to 948, 1131 to 1170, 1296 to 1331, 1451 to 1482, 1506 to 1530, 1551 to 1585, 1648 to 1695, 1786 to 1805, 2059 to 2115, 2133 to 2213, 2268 to 2310, and 2480 to 2499 from the 5′ end of the base sequence of SEQ ID NO: 1.

(19) The antisense oligonucleotide or the pharmaceutically acceptable salt thereof, or the hydrate thereof according to (18-1) or (18-2), comprising:
(i) a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502;
(ii) a base sequence having addition, deletion, or substitution of one or several bases in a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502; or
(iii) a base sequence having 90% or more sequence identity with a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502.

(20-1) The antisense oligonucleotide or the pharmaceutically acceptable salt thereof, or the hydrate thereof according to (12-1) complementary to a nucleic acid comprising at least 15 consecutive bases in a target region selected from the group consisting of positions 520 to 548, 880 to 903, 927 to 950, 1129 to 1172, 1461 to 1484, 1509 to 1532, 1549 to 1572, 1647 to 1697, 1784 to 1807, 2069 to 2117, 2131 to 2215, and 2269 to 2305 from the 5′ end of the base sequence of SEQ ID NO: 1.

(20-2) The antisense oligonucleotide or the pharmaceutically acceptable salt thereof, or the hydrate thereof according to (20-1) complementary to a nucleic acid comprising at least 15 consecutive bases in a target region selected from the group consisting of positions 522 to 546, 882 to 901, 929 to 948, 1131 to 1170, 1463 to 1482, 1511 to 1530, 1551 to 1570, 1649 to 1671, 1676 to 1695, 1786 to 1805, 2071 to 2115, 2133 to 2213, and 2271 to 2303 from the 5′ end of the base sequence of SEQ ID NO: 1.

(21) The antisense oligonucleotide or the pharmaceutically acceptable salt thereof, or the hydrate thereof according to (20-1) or (20-2), comprising:
(i) a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490;
(ii) a base sequence having addition, deletion, or substitution of one or several bases in a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490; or
(iii) a base sequence having 90% or more sequence identity with a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490.

(22) The antisense oligonucleotide or the pharmaceutically acceptable salt thereof, or the hydrate thereof according to (12-1) or (12-2) complementary to a nucleic acid comprising at least 15 consecutive bases in a target region selected from the group consisting of positions 521 to 545, 881 to 900, 1461 to 1480, 1561 to 1580, 1649 to 1670, 1676 to 1695, 2061 to 2105, 2134 to 2213, 2271 to 2299, and 2481 to 2500 from the 5′ end of the base sequence of SEQ ID NO: 1.

(23) The antisense oligonucleotide or the pharmaceutically acceptable salt thereof, or the hydrate thereof according to (20-1) or (20-2), comprising:
(i) a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402;
(ii) a base sequence having addition, deletion, or substitution of one or several bases in a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402; or
(iii) a base sequence having 90% or more sequence identity with a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402.

(24) The antisense oligonucleotide or the pharmaceutically acceptable salt thereof, or the hydrate thereof according to any one of (13), (15), (17), (19), (21), and (23), comprising the base sequence (i).

(25-1) The antisense oligonucleotide or the pharmaceutically acceptable salt thereof, or the hydrate thereof any one of (1) to (24), wherein the antisense oligonucleotide consists of 20 nucleotides.

(25-2) The antisense oligonucleotide or the pharmaceutically acceptable salt thereof, or the hydrate thereof according to any one of (1) to (24), wherein the antisense oligonucleotide consists of 15 to 20 nucleotides.

(26) The antisense oligonucleotide or the pharmaceutically acceptable salt thereof, or the hydrate thereof according to any one of (1) to (25), which reduces TDP-43 gene expression level.

(27) The antisense oligonucleotide or the pharmaceutically acceptable salt thereof, or the hydrate thereof according to any one of (1) to (26), wherein a sugar moiety and/or a phosphate bond moiety of at least one nucleotide constituting the oligonucleotide is modified.

(28-1) The antisense oligonucleotide or the pharmaceutically acceptable salt thereof, or the hydrate thereof according to (27), wherein the antisense oligonucleotide is a gapmer comprising a gap region at the center, and two wing regions adjacent to the gap region on the 5′ end side and the 3′ end side (a 5′ wing region and a 3′ wing region).

(28-2) The antisense oligonucleotide or the pharmaceutically acceptable salt thereof, or the hydrate thereof according to (28-1), wherein the antisense oligonucleotide comprises, in the 5′ to 3′ direction, a 5′ wing region of 5 nucleotides in length, a gap region of 10 nucleotides in length, and a 3′ wing region of 5 nucleotides in length.

(29) The antisense oligonucleotide or the pharmaceutically acceptable salt thereof, or the hydrate thereof according to (28-1) or (28-2), wherein bonds between nucleosides are all phosphorothioate bonds.

(30) The antisense oligonucleotide or the pharmaceutically acceptable salt thereof, or the hydrate thereof according to (28-1) or (28-2), wherein one or more of a bond between the 2nd and 3rd nucleosides, a bond between the 3rd and 4th nucleosides, and a bond between the 4th and 5th nucleosides from the 5′ side of the 5′ wing region are phosphodiester bonds, and/or one or more of a bond between the 1st and 2nd nucleosides, a bond between the 2nd and 3rd nucleosides, and a bond between the 3rd and 4th nucleosides from the 5′ side of the 3′ wing region are phosphodiester bonds, and bonds between the other nucleosides are all phosphorothioate bonds.

(31) The antisense oligonucleotide or the pharmaceutically acceptable salt thereof, or the hydrate thereof according to (28-1) or (28-2), wherein the bond between the 2nd and 3rd nucleosides, and the bond between the 4th and 5th nucleosides from the 5′ side of the 5′ wing region are phosphodiester bonds, and/or the bond between the 1st and 2nd nucleosides, and the bond between the 3rd and 4th nucleosides from the 5′ side of the 3′ wing region are phosphodiester bonds, and bonds between the other nucleosides are all phosphorothioate bonds.

(32) The antisense oligonucleotide or the pharmaceutically acceptable salt thereof, or the hydrate thereof according to (28-1) or (28-2), wherein a bond between the 1st and 2nd nucleosides from the 5′ side of the gap region is a phosphodiester bond, and bonds between the other nucleosides are all phosphorothioate bonds.

(33) The antisense oligonucleotide or the pharmaceutically acceptable salt thereof, or the hydrate thereof according to (28-1) or (28-2), wherein a bond between the 2nd and 3rd nucleosides from the 5′ side of the gap region is a phosphodiester bond, and bonds between the other nucleosides are all phosphorothioate bonds.

(34) The antisense oligonucleotide or the pharmaceutically acceptable salt thereof, or the hydrate thereof according to (28-1) or (28-2), wherein a bond between the 2nd and 3rd nucleosides, and a bond between the 4th and 5th nucleosides from the 5′ side of the 5′ wing region are phosphodiester bonds; and/or a bond between the 1st and 2nd nucleosides, and a bond between the 3rd and 4th nucleosides from the 5′ side of the 3′ wing region are phosphodiester bonds; and/or a bond between the 1st and 2nd nucleosides from the 5′ side of the gap region is a phosphodiester bond; and bonds between the other nucleosides are all phosphorothioate bonds.

(35) The antisense oligonucleotide or the pharmaceutically acceptable salt thereof, or the hydrate thereof according to (28-1) or (28-2), wherein a bond between the 2nd and 3rd nucleosides, and a bond between the 4th and 5th nucleosides from the 5′ side of the 5′ wing region are phosphodiester bonds, and/or a bond between the 1st and 2nd nucleosides, and a bond between the 3rd and 4th nucleosides from the 5′ side of the 3′ wing region are phosphodiester bonds, and/or a bond between the 2nd and 3rd nucleosides from the 5′ side of the gap region is a phosphodiester bond, and bonds between the other nucleosides are all phosphorothioate bonds.

(36) The antisense oligonucleotide or the pharmaceutically acceptable salt thereof, or the hydrate thereof according to any one of (28-1) to (35), wherein the wing and/or gap region comprises a 2′-OMe (2′-O—CH₃) group and/or a 2′-O-MOE (2′-O—CH₂CH₂OCH₃) group.

(37) The antisense oligonucleotide or the pharmaceutically acceptable salt thereof, or the hydrate thereof according to (36), wherein the 2nd nucleotide from the 5′ end of the gap region is a ribonucleotide comprising a 2′-OMe (2′-O—CH₃) group or a 2′-O-MOE (2′-O—CH₂CH₂OCH₃) group.

(38) A pharmaceutical composition comprising the antisense oligonucleotide or the pharmaceutically acceptable salt thereof, or the hydrate thereof according to any one of (1) to (37).

(39) The antisense oligonucleotide or the pharmaceutically acceptable salt thereof, or the hydrate thereof according to any one of (1) to (37), or the pharmaceutical composition according to (38), for treating and/or preventing a TDP-43 proteinopathy.

(40-1) A method for treating and/or preventing a TDP-43 proteinopathy comprising a step of administering, to a subject, the antisense oligonucleotide or the pharmaceutically acceptable salt thereof, or the hydrate thereof according to any one of (1) to (37), or the pharmaceutical composition according to (38).

(40-2) Use of the antisense oligonucleotide or the pharmaceutically acceptable salt thereof, or the hydrate thereof according to any one of (1) to (37), or the pharmaceutical composition according to (38) in production of a medicament for use in a method for treating and/or preventing a TDP-43 proteinopathy.

(40-3) The pharmaceutical composition according to (39), the method according to (40-1), or the use according to (40-2), wherein the TDP-43 proteinopathy is selected from the group consisting of amyotrophic lateral sclerosis, frontotemporal lobar degeneration, Perry syndrome, and Lewy body disease.

It is preferable that antisense oligonucleotides consisting of the following base sequences are excluded (not comprised) in the antisense oligonucleotide of the present invention.

TABLE 1

| Sequence 5′ to 3′ | SEQ ID NO: |
|---|---|
| CAGTCTTAAGATCTTTCTTG | 436 |
| CAAAGGCTCATCTTGGCTTT | 437 |
| GTGCTTAGGTTAGGCATTGG | 438 |
| ATCCATGCTTGAGCCAAAGC | 65 |
| AATATCCATTATGCACCACC | 439 |
| GTGCTTAGGTTCGGCATTGG | 440 |
| AGGTTCGGCATTGGATATAT | 441 |
| TTAGGTTCGGCATTGGATAT | 442 |
| GCTTAGGTTCGGCATTGGAT | 443 |
| CUGCACGCGCUCUCUCCUUU | 444 |
| CUCUGCACGCGCUCUCUCCU | 445 |
| GUCUCUGCACGCGCUCUCUC | 446 |
| AAGUCUCUGCACGCGCUCUC | 447 |
| CCAAGUCUCUGCACGCGCUC | 448 |
| CACCAAGUCUCUGCACGCGC | 449 |
| ACCACCAAGUCUCUGCACGC | 450 |
| GCACCACCAAGUCUCUGCAC | 451 |
| AUGCACCACCAAGUCUCUGC | 452 |
| UUAUGCACCACCAAGUCUCU | 453 |
| CAUUAUGCACCACCAAGUCU | 454 |
| UCCAUUAUGCACCACCAAGU | 455 |
| GCAAAGCACACCACAAGCUC | 456 |
| CUGCAAAGCACACCACAAGC | 457 |
| UCCUGCAAAGCACACCACAA | 458 |
| CCUCCUGCAAAGCACACCAC | 459 |
| GUCCUCCUGCAAAGCACACC | 460 |
| AAGUCCUCCUGCAAAGCACA | 461 |
| UCAAGUCCUCCUGCAAAGCA | 462 |
| CUUCAAGUCCUCCUGCAAAG | 463 |
| CAAGUCCUCCUGCAAAGCAC | 464 |
| UUCAAGUCCUCCUGCAAAGC | 465 |
| CAGTTTTATTTAAGAATTTC | 466 |
| GGGCATGCAGAATTCCTTCTAC | 467 |
| CCATCGTCTTCCGATGGTAT | 468 |
| GCUCAAGCAUGGAUUCUAA | 469 |
| CAAUCAAGGUAGUAAUAUG | 470 |
| GGGCUUCGCUACAGGAAUC | 471 |
| CAGGGUGGAUUUGGUAAUA | 472 |
| TCCACACTGAACAAACC | 473 |
| AAGGAAAAUGGAUGAGACAGA | 474 |

The present invention provides an antisense oligonucleotide targeting an TDP-43 mRNA or pre-mRNA, or a pharmaceutically acceptable salt thereof, or a hydrate thereof, and a composition or the like comprising the antisense oligonucleotide or the pharmaceutically acceptable salt thereof, or the hydrate thereof.

In a preferred embodiment of the present invention, expression of TDP-43 is inhibited through direct action on a transcriptional product of TDP-43 gene, that is, a causative gene for TDP-43 proteinopathies, and thus, a TDP-43 proteinopathy therapeutic agent with high treatment satisfaction can be provided. In the present invention, since the antisense oligonucleotide can be designed with targeting transcriptional product of a TDP-43 gene that is a causative gene for TDP-43 proteinopathies, a TDP-43 proteinopathy therapeutic agent with few side effects can be provided in a preferred embodiment of the present invention. In one embodiment of the present invention, personalized medicine based on genetic information of individual patients can be provided.

### DESCRIPTION OF EMBODIMENTS

In one embodiment, the present invention relates to an antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1. In another embodiment, the present invention relates to an antisense oligonucleotide or a pharmaceutically acceptable salt thereof, or a hydrate thereof, consisting of 15 to 30 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1. In another embodiment, the present invention relates to an antisense oligonucleotide or a pharmaceutically acceptable salt thereof, or a hydrate thereof, consisting of 15 to 25 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1.

SEQ ID NO: 1 is a sequence of an mRNA of human TDP-43 (Gen Bank: NM_007375.4). The sequence of SEQ ID NO: 1 is a base sequence comprising 4185 bases.

In one embodiment, the antisense oligonucleotide of the present invention is complementary to a nucleic acid comprising or consisting of at least 15, at least 16, at least 17, at least 18, at least 19, at least 20, and, for example, 20 consecutive bases in the target region.

As used herein, a base “complementary” to a given base means a base that forms a base pair with the intended base, and is not limited to a base that forms a Watson-Crick base pair, but also encompasses a base that forms a wobble base pair or a Hoogsteen base pair therewith. Herein, the Watson-Crick base pair means a base pair in which a hydrogen acceptor given from the position N3 of a pyrimidine base occupies the position Ni of a purine base in a hydrogen bond between adenine and thymine, between adenine and uracil, or between guanine and cytosine. The wobble base pair means a base pair that forms a hydrogen bond between guanine and uracil, between inosine and uracil, between inosine and adenine, or between inosine and cytosine. The Hoogsteen base pair means a base pair in which a hydrogen acceptor given from the position N3 of a pyrimidine base occupies the position N7 of a purine base in a hydrogen bond between adenine and thymine, between adenine and uracil, or between guanine and cytosine.

An example of an antisense oligonucleotide “complementary” to a given nucleic acid includes an antisense oligonucleotide that can hybridize under stringent conditions to the nucleic acid. As used herein, the term “stringent conditions” may be any of low stringent conditions, moderate stringent conditions, and high stringent conditions. The term “low stringent conditions” is conditions of, for example, 5×SSC, 5×Denhardt's solution, 0.5% SDS, 50% formamide at 32° C. The term “moderate stringent conditions” is conditions of, for example, 5×SSC, 5×Denhardt's solution, 0.5% SDS, 50% formamide at 42° C., or 5×SSC, 1% SDS, 50 mM Tris-HCl (pH 7.5), 50% formamide at 42° C. The term “high stringent conditions” is conditions of, for example, 5×SSC, 5×Denhardt's solution, 0.5% SDS, 50% formamide at 50° C., or 0.2×SSC, 0.1% SDS at 65° C. Under these conditions, base sequences with higher sequence identity are expected to be obtained efficiently at higher temperatures. Multiple factors are, however, involved in hybridization stringency including temperature, probe concentration, probe length, ionic strength, time, salt concentration and others, and those skilled in the art may appropriately select these factors to achieve similar stringency.

When commercially available kits are used for hybridization, for example, an AlkPhos Direct Labelling and Detection System (GE Healthcare) may be used. In this case, according to the attached protocol, after incubation with a labeled probe overnight, the membrane can be washed with a primary wash buffer containing 0.1% (w/v) SDS at 55° C., thereby detecting hybridization. Alternatively, when a probe is labeled with digoxigenin (DIG) using a commercially available reagent (e.g., a PCR Labelling Mix (Roche Diagnostics)) in producing the probe based on a target sequence, hybridization can be detected with a DIG Nucleic Acid Detection Kit (Roche Diagnostics) or the like.

As used herein, the identity between base sequences may be determined using algorithm BLAST (Basic Local Alignment Search Tool) by Karlin and Altschul (Proc. Natl. Acad. Sci. USA, 1990, Vol. 87, pp. 2264-2268; Proc. Natl. Acad. Sci. USA, 1993, Vol. 90, pp. 5873-5877). Programs called BLASTN and BLASTX based on the BLAST algorithm have been developed (Altschul S F, et al.,: J. Mol. Biol. 1990, Vol. 215, pp. 403-410). When a base sequence is analyzed using BLASTN, the parameters are, for example, score=100 and wordlength=12. When BLAST and Gapped BLAST programs are used, the default parameters for each program are employed.

An antisense oligonucleotide of one embodiment may be, for example, 15 or more nucleotides in length, 16 or more nucleotides in length, 17 or more nucleotides in length, 18 or more nucleotides in length, 19 or more nucleotides in length, 20 or more nucleotides in length, 21 or more nucleotides in length, or 22 or more nucleotides in length, and may be 22 or less nucleotides in length, 21 or less nucleotides in length, 20 or less nucleotides in length, 19 or less nucleotides in length, 18 or less nucleotides in length, 17 or less nucleotides in length, 16 or less nucleotides in length, or 15 nucleotides in length. The antisense oligonucleotide of the one embodiment may consist of 15 to 22 nucleotides, 15 to 21 nucleotides, 15 to 20 nucleotides, 15 to 19 nucleotides, 15 to 18 nucleotides, 15 to 17 nucleotides, 15 to 16 nucleotides, 18 to 22 nucleotides, 19 to 21 nucleotides, and for example, 20 nucleotides. The antisense oligonucleotide of the one embodiment may be, for example, 15, 16, 17, 18, 19, 20, 21, or 22 nucleotides in length. Besides, an antisense oligonucleotide of one embodiment may have a nucleotide length of NLa to NLb. Here, NLa and NLb are natural numbers of 15 to 22, and NLa<NLb.

An antisense oligonucleotide of another embodiment may be 23 or more nucleotides in length, 24 or more nucleotides in length, 25 or more nucleotides in length, 26 or more nucleotides in length, 27 or more nucleotides in length, 28 or more nucleotides in length, 29 or more nucleotides in length, or 30 or more nucleotides in length, and 30 or less nucleotides in length, 29 or less nucleotides in length, 28 or less nucleotides in length, 27 or less nucleotides in length, 26 or less nucleotides in length, 25 or less nucleotides in length, 24 or less nucleotides in length, or 23 or less nucleotides in length. The antisense oligonucleotide of the another embodiment may be, for example, 23 to 30 nucleotides, 23 to 25 nucleotides (for example, 23 to 24 nucleotides, or 23 nucleotides). The antisense oligonucleotide of another embodiment may be, for example, 23, 24, 25, 26, 27, 28, 29, or 30 nucleotides in length. Besides, the antisense oligonucleotide of another embodiment may have a nucleotide length of NLc to NLd. Here, NLc and NLd are natural numbers of 23 to 30, and NLc<NLd.

Examples of the pharmaceutically acceptable salt of the antisense oligonucleotide of the present invention are alkali metal salts such as salts of sodium, potassium and lithium; alkaline earth metal salts such as salts of calcium and magnesium; metal salts such as salts of aluminum, iron, zinc, copper, nickel, cobalt, etc.; ammonium salts; organic amine salts such as salts of t-octylamine, dibenzylamine, morpholine, glucosamine, phenylglycine alkyl ester, ethylenediamine, N-methylglucamine, guanidine, diethylamine, triethylamine, dicyclohexylamine, N,N′-dibenzylethylenediamine, chloroprocaine, procaine, diethanolamine, N-benzylphenethylamine, piperazine, tetramethylammonium, and tris(hydroxymethyl)aminomethane; hydrohalide salts such as salts of hydrofluorates, hydrochlorides, hydrobromides, and hydroiodides; inorganic acid salts such as nitrates, perchlorates, sulfates, and phosphates; lower alkane sulfonates such as methanesulfonates, trifluoromethanesulfonates and ethanesulfonates; arylsulfonates such as benzenesulfonates, and p-toluenesulfonates; organic acid salts such as acetates, malates, fumarates, succinates, citrates, tartrates, oxalates, and maleates; and amino acid salts such as salts of glycine, lysine, arginine, ornithine, glutamic acid, and aspartic acid. Preferable examples of the pharmaceutically acceptable salt of the antisense oligonucleotide of the present invention include a triethylamine salt and a sodium salt. These salts may be produced by known methods. Alternatively, the antisense oligonucleotide of the present invention may be in the form of a hydrate thereof.

The antisense oligonucleotide of the present invention is composed of nucleotides as constituent units, and such nucleotides may be any of ribonucleotides, deoxyribonucleotides, and modified nucleotides.

The modified nucleotide refers to one fully or partly modified in a nucleobase, a sugar moiety and a phosphate bond moiety that constitute the ribonucleotide or deoxyribonucleotide.

The nucleobase includes, for example, adenine, guanine, hypoxanthine, cytosine, thymine, uracil, or modified bases thereof. Examples of such modified bases include, but are not limited to, pseudouracil, 3-methyluracil, dihydrouracil, 5-alkylcytosines (e.g., 5-methylcytosine), 5-alkyluracils (e.g., 5-ethyluracil), 5-halouracils (e.g., 5-bromouracil), 6-azapyrimidine, 6-alkylpyrimidines (e.g., 6-methyluracil), 2-thiouracil, 4-thiouracil, 4-acetylcytosine, 5-(carboxyhydroxymethyl) uracil, 5-carboxymethylaminomethyl-2-thiouracil, 5-carboxymethylaminomethyluracil, 1-methyladenine, 1-methylhypoxanthine, 2,2-dimethylguanine, 3-methylcytosine, 2-methyladenine, 2-methylguanine, N6-methyladenine, 7-methylguanine, 5-methoxyaminomethyl-2-thiouracil, 5-methylaminomethyluracil, 5-methylcarbonylmethyluracil, 5-methyloxyuracil, 5-methyl-2-thiouracil, 2-methylthio-N6-isopentenyladenine, uracil-5-oxyacetic acid, 2-thiocytosine, purine, 2,6-diaminopurine, 2-aminopurine, isoguanine, indole, imidazole, and xanthine.

As used herein, thymine “T” and uracil “U” are interchangeable with each other. Neither “T” nor “U” essentially influences the activity of the antisense oligonucleotide of the present invention, and therefore, in the base sequences shown in this specification, cases where “T” is “U” are also included, and these are indicated by the same sequence number. Besides, as used herein, a sequence comprising a modified base and a sequence not comprising the modified base are represented by the same SEQ ID NO. For example, “cytosine” and “methylcytosine” are interchangeable with each other, and cases where “cytosine” is “methylcytosine” are also included, and these are indicated by the same sequence number

Modification of the sugar moiety may include, for example, modifications at the 2′-position of ribose, and modifications of the other portions of the sugar. The modification at the 2′-position of ribose includes a modification of replacing the 2′-OH of ribose with —OR, —OROR, —R, —R′OR, —SH, —SR, —NH₂, —NHR, —NR₂, —N₃, —CN, —F, —Cl, —Br or —I, for example, —OMe(—O—CH₃) or —O— methoxyethyl (—O-MOE: —O—CH₂CH₂OCH₃). Here, R represents an alkyl or an aryl, and R′ represents an alkylene.

The modification for the other portions of the sugar includes, for example, replacement of 0 at the 4′-position of ribose or deoxyribose with S, bridging between 2′- and 4′-positions of the sugar, such as LNA (locked nucleic acid) or ENA (2′-O,4′-C-ethylene-bridged nucleic acids), but is not limited thereto. Besides, the crosslinking site may be modified with urea or guanidine.

The modification for the phosphate bond moiety includes, for example, a modification of replacing phosphodiester bond with a phosphorothioate bond, a phosphorodithioate bond, an alkyl phosphonate bond, a phosphoramidate bond, or a boranophosphate bond (cf., e.g., Enya et al.,: Bioorganic & Medicinal Chemistry, 2008, 18, pp. 9154-9160) (cf., e.g., Japan Domestic Re-Publications of PCT Application Nos. 2006/129594 and 2006/038608).

As used herein, the alkyl is preferably a straight or branched alkyl having 1 to 6 carbon atoms. Specific examples include methyl, ethyl, n-propyl, isopropyl, n-butyl, isobutyl, sec-butyl, tert-butyl, n-pentyl, isopentyl, neopentyl, tert-pentyl, n-hexyl and isohexyl. The alkyl may optionally be substituted, and examples of such substituents are a halogen, an alkoxy, a cyano and a nitro. The alkyl may be substituted with 1 to 3 substituents.

As used herein, examples of the halogen include fluorine, chlorine, bromine, and iodine.

As used herein, the alkoxy is a straight or branched alkoxy having 1 to 6 carbon atoms such as methoxy, ethoxy, n-propoxy, isopropoxy, n-butoxy, isobutoxy, sec-butoxy, tert-butoxy, n-pentyloxy, isopentyloxy, n-hexyloxy, and isohexyloxy. Among others, an alkoxy having 1 to 3 carbon atoms is preferred.

As used herein, the aryl is preferably an aryl having 6 to 10 carbon atoms. Specific examples include phenyl, α-naphthyl, and β-naphthyl. Among others, phenyl is preferred. The aryl may optionally be substituted, and examples of such substituents include an alkyl, a halogen, an alkoxy, a cyano and a nitro. The aryl may be substituted with 1 to 3 substituents.

As used herein, the alkylene is preferably a straight or branched alkylene having 1 to 6 carbon atoms. Specific examples include methylene, ethylene, trimethylene, tetramethylene, pentamethylene, hexamethylene, 2-(ethyl) trimethylene, and 1-(methyl) tetramethylene.

In one embodiment, the antisense oligonucleotide of the present invention is a gapmer comprising a gap region at the center, and two wing regions adjacent to the gap region on the 5′ end side and the 3′ end side (referred to also as the 5′ wing region and the 3′ wing region, respectively).

The gap region is a region recognized by RNase H, and constituted, in at least the 5′ end or 3′ end, by a deoxyribonucleotide not modified in the sugar moiety. The gap region may be fully constituted by a deoxyribonucleotide not modified in the sugar moiety, but may comprise one or more nucleotide modified in the sugar moiety. For example, the gap region may comprise one nucleotide modified in the sugar moiety at position 2 from the 5′ end side of the gap region, and all the other may be deoxyribonucleotides not modified in the sugar moiety. The modified nucleotide is, for example, a ribonucleotide having modification at the 2′-position of ribose, and may comprise a 2′-OMe group and/or a 2′-O-MOE group. In one embodiment, the 2nd nucleotide from the 5′ end of the gap region is a ribonucleotide having a 2′-OMe (2′-O—CH₃) group or a 2′-O-MOE (2′-O—CH₂CH₂OCH₃) group.

The wing region comprises at least one modified nucleotide, and is, for example, fully constituted by a modified nucleotide (e.g., a ribonucleotide having modification at the 2′-position of ribose). In one embodiment, each of nucleosides of the 5′ wing region and the 3′ wing region comprises at least one, for example, two or more, three or more, four or more, or five or more modified sugar moieties, such as a 2′-OMe group and/or a 2′-O-MOE group, and for example, all nucleosides of the 5′ wing region and the 3′ wing region may comprise a 2′-OMe group and/or a 2′-O-MOE group, for example a 2′-O-MOE group. Besides, the nucleosides of the 5′ wing region and the 3′ wing region may comprise a modification in the base moiety, and may comprise, for example, at least one methylcytosine, and for example, all cytosines comprised therein may be methylcytosine.

The length of the gap region is not limited, and may be, for example, 5 to 15, 8 to 12, 9 to 11, or 10 bases in length. The lengths of the 5′ wing region and the 3′ wing region are not limited, and may be, for example, independently 2 to 10, 3 to 8, 4 to 6, or 5 bases in length. In one embodiment, the length of the gap region is 10 bases in length, and the lengths of the 5′ wing region and the 3′ wing region are respectively 5 bases in length, and such a gapmer is herein referred to as the “5-10-5 gapmer”.

The antisense oligonucleotide of the present invention may be a gapmer comprising a base sequence having addition, deletion, or substitution of 1 to 2 bases in the wing region, or a gapmer comprising a base sequence having addition, deletion, or substitution of 1 to 4 bases in the gap region in any of gapmers of Examples described herein.

The antisense oligonucleotide of the present invention may be a 5-10-5 gapmer comprising a base sequence obtained by substituting 1 to 2 bases in the wing region, or a 5-10-5 gapmer comprising a base sequence obtained by substituting 1 to 4 bases in the gap region in any of gapmers of Examples described herein. The antisense oligonucleotide of the present invention may be a 5-10-5 gapmer having at least 3 consecutive bases of the wing region of gapmers of Examples described herein, or a 5-10-5 gapmer having at least 6 consecutive bases of the gap region of gapmers of Examples described herein.

In one embodiment, the gapmer of the present invention comprises one or more modifications of phosphate bond moieties, e.g., phosphorothioate bonds, and for example, one or more, two or more, three or more, four or more, five or more, ten or more, fifteen or more, and for example, all of bonds between nucleotides may be phosphorothioate bonds. In one embodiment, the gapmer of the present invention does not comprise modification of a phosphate bond moiety, and all of bonds between nucleotides may be phosphate bonds.

In one embodiment, in the gapmer of the present invention, one or more of a bond between the 2nd and 3rd nucleosides, a bond between the 3rd and 4th nucleosides, and a bond between the 4th and 5th nucleosides from the 5′ side of the 5′ wing region may be phosphodiester bonds, and/or one or more of a bond between the 1st and 2nd nucleosides, a bond between the 2nd and 3rd nucleosides, and a bond between the 3rd and 4th nucleosides from the 5′ side of the 3′ wing region may be phosphodiester bonds, and bonds between the other nucleosides may be all phosphorothioate bonds.

In one embodiment, in the gapmer of the present invention, the bond between the 2nd and 3rd nucleosides, and the bond between the 4th and 5th nucleosides from the 5′ side of the 5′ wing region may be phosphodiester bonds, and/or the bond between the 1st and 2nd nucleosides, and the bond between the 3rd and 4th nucleosides from the 5′ side of the 3′ wing region may be phosphodiester bonds, and bonds between the other nucleosides may be all phosphorothioate bonds.

In one embodiment, in the gapmer of the present invention, the bond between the 1st and 2nd nucleosides from the 5′ side of the gap region may be a phosphodiester bond, and bonds between the other nucleosides may be all phosphorothioate bonds.

In one embodiment, in the gapmer of the present invention, the bond between the 2nd and 3rd nucleosides from the 5′ side of the gap region may be a phosphodiester bond, and bonds between the other nucleosides may be all phosphorothioate bonds.

In one embodiment, in the gapmer of the present invention, the bond between the 2nd and 3rd nucleosides, and the bond between the 4th and 5th nucleosides from the 5′ side of the 5′ wing region may be phosphodiester bonds, and/or the bond between the 1st and 2nd nucleosides, and the bond between the 3rd and 4th nucleosides from the 5′ side of the 3′ wing region may be phosphodiester bonds, and/or the bond between the 1st and 2nd nucleosides from the 5′ side of the gap region may be a phosphodiester bond, and bonds between the other nucleosides may be all phosphorothioate bonds.

In one embodiment, in the gapmer of the present invention, the bond between the 2nd and 3rd nucleosides, and the bond between the 4th and 5th nucleosides from the 5′ side of the 5′ wing region may be phosphodiester bonds, and/or the bond between the 1st and 2nd nucleosides, and the bond between the 3rd and 4th nucleosides from the 5′ side of the 3′ wing region may be phosphodiester bonds, and/or the bond between the 2nd and 3rd nucleosides from the 5′ side of the gap region may be a phosphodiester bond, and bonds between the other nucleosides may be all phosphorothioate bonds.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 102, 159 to 842, 879 to 1822, and 1874 to 4182 from the 5′ end of the base sequence of SEQ ID NO: 1.

In one embodiment, from the antisense oligonucleotide of the present invention, an antisense oligonucleotide or a pharmaceutically acceptable salt thereof, or a hydrate thereof having a sequence consisting of a base sequence selected from the group consisting of SEQ ID NOs: 65, 436 to 440, 444 to 449, 451 to 452, and 454 to 474 is preferably excluded,
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is preferably excluded, and/or
an antisense oligonucleotide or a pharmaceutically acceptable salt thereof, or a hydrate thereof having a sequence consisting of a base sequence of SEQ ID NO: 453, and having ENA: 2′-O,4′-C-ethylene bridged nucleic acid at each of positions 1 to 2, 4, 6, 8 to 9, 11 to 12, and 16 to 20 and a ribonucleotide comprising a 2′-OMe group at each of positions 3, 5, 7, 10, and 13 to 15 from the 5′ end of the base sequence of SEQ ID NO: 453 is preferably excluded. Besides, in one embodiment, an unmodified antisense oligonucleotide or pharmaceutically acceptable salt thereof, or a hydrate thereof having the base sequence of SEQ ID NO: 450 or SEQ ID NO: 453 is preferably excluded.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 100, 161 to 840, 881 to 1820, and 1876 to 4180 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 20, 21 to 40, 41 to 60, 61 to 80, 81 to 100, 161 to 180, 181 to 200, 201 to 220, 221 to 240, 241 to 260, 261 to 280, 281 to 300, 301 to 320, 321 to 340, 341 to 360, 361 to 380, 381 to 400, 401 to 420, 421 to 440, 441 to 460, 461 to 480, 481 to 500, 501 to 520, 521 to 540, 541 to 560, 561 to 580, 574 to 593, 581 to 600, 601 to 620, 621 to 640, 641 to 660, 661 to 680, 681 to 700, 701 to 720, 721 to 740, 741 to 760, 761 to 780, 781 to 800, 801 to 820, 821 to 840, 881 to 900, 901 to 920, 921 to 940, 933 to 952, 941 to 960, 961 to 980, 981 to 1000, 1001 to 1020, 1021 to 1040, 1041 to 1060, 1061 to 1080, 1081 to 1100, 1093 to 1112, 1101 to 1120, 1121 to 1140, 1141 to 1160, 1161 to 1180, 1181 to 1200, 1201 to 1220, 1221 to 1240, 1241 to 1260, 1261 to 1280, 1281 to 1300, 1301 to 1320, 1321 to 1340, 1341 to 1360, 1361 to 1380, 1381 to 1400, 1394 to 1413, 1401 to 1420, 1421 to 1440, 1441 to 1460, 1461 to 1480, 1476 to 1495, 1481 to 1500, 1501 to 1520, 1521 to 1540, 1541 to 1560, 1561 to 1580, 1581 to 1600, 1601 to 1620, 1621 to 1640, 1641 to 1660, 1661 to 1680, 1681 to 1700, 1701 to 1720, 1721 to 1740, 1741 to 1760, 1761 to 1780, 1781 to 1800, 1801 to 1820, 1876 to 1895, 1881 to 1900, 1901 to 1920, 1916 to 1935, 1921 to 1940, 1941 to 1960, 1961 to 1980, 1981 to 2000, 2001 to 2020, 2021 to 2040, 2033 to 2052, 2041 to 2060, 2061 to 2080, 2073 to 2092, 2081 to 2100, 2101 to 2120, 2121 to 2140, 2141 to 2160, 2153 to 2172, 2161 to 2180, 2181 to 2200, 2195 to 2214, 2201 to 2220, 2221 to 2240, 2241 to 2260, 2261 to 2280, 2275 to 2294, 2281 to 2300, 2301 to 2320, 2321 to 2340, 2341 to 2360, 2361 to 2380, 2381 to 2400, 2401 to 2420, 2421 to 2440, 2441 to 2460, 2448 to 2467, 2461 to 2480, 2481 to 2500, 2501 to 2520, 2521 to 2540, 2528 to 2547, 2541 to 2560, 2561 to 2580, 2581 to 2600, 2601 to 2620, 2621 to 2640, 2641 to 2660, 2647 to 2666, 2661 to 2680, 2681 to 2700, 2686 to 2705, 2701 to 2720, 2721 to 2740, 2733 to 2752, 2741 to 2760, 2761 to 2780, 2766 to 2785, 2781 to 2800, 2801 to 2820, 2807 to 2826, 2821 to 2840, 2841 to 2860, 2847 to 2866, 2861 to 2880, 2881 to 2900, 2887 to 2906, 2901 to 2920, 2921 to 2940, 2941 to 2960, 2961 to 2980, 2981 to 3000, 3001 to 3020, 3005 to 3024, 3021 to 3040, 3041 to 3060, 3061 to 3080, 3081 to 3100, 3101 to 3120, 3121 to 3140, 3141 to 3160, 3161 to 3180, 3181 to 3200, 3201 to 3220, 3221 to 3240, 3241 to 3260, 3261 to 3280, 3281 to 3300, 3301 to 3320, 3321 to 3340, 3341 to 3360, 3361 to 3380, 3381 to 3400, 3401 to 3420, 3421 to 3440, 3441 to 3460, 3461 to 3480, 3481 to 3500, 3501 to 3520, 3521 to 3540, 3541 to 3560, 3561 to 3580, 3581 to 3600, 3601 to 3620, 3621 to 3640, 3641 to 3660, 3661 to 3680, 3681 to 3700, 3701 to 3720, 3721 to 3740, 3741 to 3760, 3761 to 3780, 3781 to 3800, 3801 to 3820, 3821 to 3840, 3841 to 3860, 3861 to 3880, 3881 to 3900, 3901 to 3920, 3921 to 3940, 3941 to 3960, 3961 to 3980, 3981 to 4000, 4001 to 4020, 4021 to 4040, 4041 to 4060, 4061 to 4080, 4081 to 4100, 4101 to 4120, 4121 to 4140, 4141 to 4160, and 4161 to 4180 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 2 to 64, 66 to 224, 422 to 423, 426 to 427, and 432;
(ii) a base sequence having addition, deletion, or substitution of one or several bases in a base sequence selected from the group consisting of SEQ ID NOs: 2 to 64, 66 to 224, 422 to 423, 426 to 427, and 432; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 2 to 64, 66 to 224, 422 to 423, 426 to 427, and 432,
e.g., the base sequence (i).

As used herein, the term “several” used in the phrase of “base sequence having addition, deletion, or substitution of one or several bases” means, two, three, four, five, six, seven, eight, nine, or ten.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 102, 159 to 622, 639 to 842, 879 to 1442, 1459 to 1497, 1499 to 1522, 1539 to 1702, 1719 to 1762, 1779 to 1802, 1874 to 1937, 1939 to 2302, 2319 to 3162, 3199 to 3242, 3279 to 3382, 3399 to 3482, 3539 to 3562, 3579 to 3602, 3619 to 3662, 3679 to 3702, and 3759 to 4182 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 100, 161 to 620, 641 to 840, 881 to 1440, 1461 to 1495, 1501 to 1520, 1541 to 1700, 1721 to 1760, 1781 to 1800, 1876 to 1935, 1941 to 2300, 2321 to 3160, 3201 to 3240, 3281 to 3380, 3401 to 3480, 3541 to 3560, 3581 to 3600, 3621 to 3660, 3681 to 3700, and 3761 to 4180 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 1 to 20, 21 to 40, 41 to 60, 61 to 80, 81 to 100, 161 to 180, 181 to 200, 201 to 220, 221 to 240, 241 to 260, 261 to 280, 281 to 300, 301 to 320, 321 to 340, 341 to 360, 361 to 380, 381 to 400, 401 to 420, 421 to 440, 441 to 460, 461 to 480, 481 to 500, 501 to 520, 521 to 540, 541 to 560, 561 to 580, 574 to 593, 581 to 600, 601 to 620, 641 to 660, 661 to 680, 681 to 700, 701 to 720, 721 to 740, 741 to 760, 761 to 780, 781 to 800, 801 to 820, 821 to 840, 881 to 900, 901 to 920, 921 to 940, 933 to 952, 941 to 960, 961 to 980, 981 to 1000, 1001 to 1020, 1021 to 1040, 1041 to 1060, 1061 to 1080, 1081 to 1100, 1093 to 1112, 1101 to 1120, 1121 to 1140, 1141 to 1160, 1161 to 1180, 1181 to 1200, 1201 to 1220, 1221 to 1240, 1241 to 1260, 1261 to 1280, 1281 to 1300, 1301 to 1320, 1321 to 1340, 1341 to 1360, 1361 to 1380, 1381 to 1400, 1394 to 1413, 1401 to 1420, 1421 to 1440, 1461 to 1480, 1476 to 1495, 1501 to 1520, 1541 to 1560, 1561 to 1580, 1581 to 1600, 1601 to 1620, 1621 to 1640, 1641 to 1660, 1661 to 1680, 1681 to 1700, 1721 to 1740, 1741 to 1760, 1781 to 1800, 1876 to 1895, 1881 to 1900, 1901 to 1920, 1916 to 1935, 1941 to 1960, 1961 to 1980, 1981 to 2000, 2001 to 2020, 2021 to 2040, 2041 to 2060, 2061 to 2080, 2073 to 2092, 2081 to 2100, 2101 to 2120, 2121 to 2140, 2141 to 2160, 2153 to 2172, 2161 to 2180, 2181 to 2200, 2195 to 2214, 2201 to 2220, 2221 to 2240, 2241 to 2260, 2261 to 2280, 2275 to 2294, 2281 to 2300, 2321 to 2340, 2341 to 2360, 2361 to 2380, 2381 to 2400, 2401 to 2420, 2421 to 2440, 2441 to 2460, 2448 to 2467, 2461 to 2480, 2481 to 2500, 2501 to 2520, 2521 to 2540, 2528 to 2547, 2541 to 2560, 2561 to 2580, 2581 to 2600, 2601 to 2620, 2621 to 2640, 2641 to 2660, 2647 to 2666, 2661 to 2680, 2681 to 2700, 2686 to 2705, 2701 to 2720, 2721 to 2740, 2741 to 2760, 2761 to 2780, 2766 to 2785, 2781 to 2800, 2801 to 2820, 2807 to 2826, 2821 to 2840, 2841 to 2860, 2861 to 2880, 2881 to 2900, 2901 to 2920, 2921 to 2940, 2941 to 2960, 2961 to 2980, 2981 to 3000, 3001 to 3020, 3005 to 3024, 3021 to 3040, 3041 to 3060, 3061 to 3080, 3081 to 3100, 3101 to 3120, 3121 to 3140, 3141 to 3160, 3201 to 3220, 3221 to 3240, 3281 to 3300, 3301 to 3320, 3321 to 3340, 3341 to 3360, 3361 to 3380, 3401 to 3420, 3421 to 3440, 3441 to 3460, 3461 to 3480, 3541 to 3560, 3581 to 3600, 3621 to 3640, 3641 to 3660, 3681 to 3700, 3761 to 3780, 3781 to 3800, 3801 to 3820, 3821 to 3840, 3841 to 3860, 3861 to 3880, 3881 to 3900, 3901 to 3920, 3921 to 3940, 3941 to 3960, 3961 to 3980, 3981 to 4000, 4001 to 4020, 4021 to 4040, 4041 to 4060, 4061 to 4080, 4081 to 4100, 4101 to 4120, 4121 to 4140, 4141 to 4160, and 4161 to 4180 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224;
(ii) a base sequence having addition, deletion, or substitution of one or several bases in a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224,
e.g., the base sequence (i).

In one embodiment, the antisense oligonucleotide of the present invention is selected from those having a high ratio of suppressing TDP-43 gene expression level, measured by a method described in Example 3 of the present specification, obtained in administration to A204 cell. For example, it may be selected from those having a ratio (average value), to the TDP-43 gene expression level obtained in administration of ANT-10 as an antisense oligonucleotide used for comparison, of 4.12 or less (here, “average value” means an average value of a result obtained by using a primer set for detecting exon 2-3 (RNA expression level (exon 2-3)), and a result obtained by using a primer set for detecting exon 5-6 (RNA expression level (exon 5-6))). Examples of such antisense oligonucleotides include antisense oligonucleotides complementary to a nucleic acid comprising at least 15, for example, at least 16, 17, 18, 19, or 20 consecutive bases in a target region selected from the group consisting of positions 19 to 42, 79 to 102, 159 to 182, 199 to 622, 639 to 662, 679 to 762, 779 to 802, 819 to 842, 879 to 1002, 1019 to 1402, 1419 to 1442, 1459 to 1497, 1499 to 1522, 1559 to 1642, 1659 to 1702, 1779 to 1802, 1874 to 1902, 1914 to 1937, 1959 to 2002, 2019 to 2302, 2319 to 2342, 2359 to 2802, 2819 to 3142, 3199 to 3222, 3299 to 3322, 3339 to 3362, 3399 to 3422, 3799 to 3842, 3879 to 3982, 4019 to 4042, and 4119 to 4142 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 21 to 40, 81 to 100, 161 to 180, 201 to 620, 641 to 660, 681 to 760, 781 to 800, 821 to 840, 881 to 1000, 1021 to 1400, 1421 to 1440, 1461 to 1495, 1501 to 1520, 1561 to 1640, 1661 to 1700, 1781 to 1800, 1876 to 1900, 1916 to 1935, 1961 to 2000, 2021 to 2300, 2321 to 2340, 2361 to 2800, 2821 to 3140, 3201 to 3220, 3301 to 3320, 3341 to 3360, 3401 to 3420, 3801 to 3840, 3881 to 3980, 4021 to 4040, and 4121 to 4140 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 21 to 40, 81 to 100, 161 to 180, 201 to 220, 221 to 240, 241 to 260, 261 to 280, 281 to 300, 301 to 320, 321 to 340, 341 to 360, 361 to 380, 381 to 400, 401 to 420, 421 to 440, 441 to 460, 461 to 480, 481 to 500, 501 to 520, 521 to 540, 541 to 560, 561 to 580, 574 to 593, 581 to 600, 601 to 620, 641 to 660, 681 to 700, 701 to 720, 721 to 740, 741 to 760, 781 to 800, 821 to 840, 881 to 900, 901 to 920, 921 to 940, 933 to 952, 941 to 960, 961 to 980, 981 to 1000, 1021 to 1040, 1041 to 1060, 1061 to 1080, 1081 to 1100, 1093 to 1112, 1101 to 1120, 1121 to 1140, 1141 to 1160, 1161 to 1180, 1181 to 1200, 1201 to 1220, 1221 to 1240, 1241 to 1260, 1261 to 1280, 1281 to 1300, 1301 to 1320, 1321 to 1340, 1341 to 1360, 1361 to 1380, 1381 to 1400, 1421 to 1440, 1461 to 1480, 1476 to 1495, 1501 to 1520, 1561 to 1580, 1581 to 1600, 1601 to 1620, 1621 to 1640, 1661 to 1680, 1681 to 1700, 1781 to 1800, 1876 to 1895, 1881 to 1900, 1916 to 1935, 1961 to 1980, 1981 to 2000, 2021 to 2040, 2041 to 2060, 2061 to 2080, 2073 to 2092, 2081 to 2100, 2101 to 2120, 2121 to 2140, 2141 to 2160, 2153 to 2172, 2161 to 2180, 2181 to 2200, 2195 to 2214, 2201 to 2220, 2221 to 2240, 2241 to 2260, 2261 to 2280, 2275 to 2294, 2281 to 2300, 2321 to 2340, 2361 to 2380, 2381 to 2400, 2401 to 2420, 2421 to 2440, 2441 to 2460, 2448 to 2467, 2461 to 2480, 2481 to 2500, 2501 to 2520, 2521 to 2540, 2528 to 2547, 2541 to 2560, 2561 to 2580, 2581 to 2600, 2601 to 2620, 2621 to 2640, 2641 to 2660, 2647 to 2666, 2661 to 2680, 2681 to 2700, 2701 to 2720, 2721 to 2740, 2741 to 2760, 2761 to 2780, 2781 to 2800, 2821 to 2840, 2841 to 2860, 2861 to 2880, 2881 to 2900, 2901 to 2920, 2921 to 2940, 2941 to 2960, 2961 to 2980, 2981 to 3000, 3001 to 3020, 3021 to 3040, 3041 to 3060, 3061 to 3080, 3081 to 3100, 3101 to 3120, 3121 to 3140, 3201 to 3220, 3301 to 3320, 3341 to 3360, 3401 to 3420, 3801 to 3820, 3821 to 3840, 3881 to 3900, 3901 to 3920, 3921 to 3940, 3941 to 3960, 3961 to 3980, 4021 to 4040, and 4121 to 4140 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222;
(ii) a base sequence having addition, deletion, or substitution of one or several bases in a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222,
e.g., the base sequence (i).

In one embodiment, the antisense oligonucleotide of the present invention is selected from those having a high ratio of suppressing the TDP-43 gene expression level, measured by the method described in Example 3 of the present specification, obtained in administration to A204 cell. For example, it may be selected from those having a ratio (average value), to the TDP-43 gene expression level obtained in administration of ANT-10 as an antisense oligonucleotide used for comparison, of 3 or less (here, “average value” means an average value of a result obtained by using a primer set for detecting exon 2-3 (RNA expression level (exon 2-3)), and a result obtained by using a primer set for detecting exon 5-6 (RNA expression level (exon 5-6))). Examples of such antisense oligonucleotides include antisense oligonucleotides complementary to a nucleic acid comprising at least 15, for example, at least 16, 17, 18, 19, or 20 consecutive bases in a target region selected from the group consisting of positions 19 to 42, 159 to 182, 239 to 282, 319 to 342, 359 to 482, 499 to 602, 639 to 662, 679 to 762, 779 to 802, 819 to 842, 879 to 902, 919 to 1002, 1039 to 1342, 1359 to 1382, 1419 to 1442, 1459 to 1482, 1499 to 1522, 1559 to 1642, 1659 to 1702, 1779 to 1802, 1879 to 1902, 1914 to 1937, 1979 to 2002, 2019 to 2042, 2059 to 2302, 2319 to 2342, 2359 to 2422, 2446 to 2542, 2559 to 2622, 2639 to 2702, 2939 to 2962, 3019 to 3042, 3119 to 3142, and 3199 to 3222 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 21 to 40, 161 to 180, 241 to 280, 321 to 340, 361 to 480, 501 to 600, 641 to 660, 681 to 760, 781 to 800, 821 to 840, 881 to 900, 921 to 1000, 1041 to 1340, 1361 to 1380, 1421 to 1440, 1461 to 1480, 1501 to 1520, 1561 to 1640, 1661 to 1700, 1781 to 1800, 1881 to 1900, 1916 to 1935, 1981 to 2000, 2021 to 2040, 2061 to 2300, 2321 to 2340, 2361 to 2420, 2448 to 2540, 2561 to 2620, 2641 to 2700, 2941 to 2960, 3021 to 3040, 3121 to 3140, and 3201 to 3220 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 21 to 40, 161 to 180, 241 to 260, 261 to 280, 321 to 340, 361 to 380, 381 to 400, 401 to 420, 421 to 440, 441 to 460, 461 to 480, 501 to 520, 521 to 540, 541 to 560, 561 to 580, 574 to 593, 581 to 600, 641 to 660, 681 to 700, 701 to 720, 721 to 740, 741 to 760, 781 to 800, 821 to 840, 881 to 900, 921 to 940, 933 to 952, 941 to 960, 961 to 980, 981 to 1000, 1041 to 1060, 1061 to 1080, 1081 to 1100, 1093 to 1112, 1101 to 1120, 1121 to 1140, 1141 to 1160, 1161 to 1180, 1181 to 1200, 1201 to 1220, 1221 to 1240, 1241 to 1260, 1261 to 1280, 1281 to 1300, 1301 to 1320, 1321 to 1340, 1361 to 1380, 1421 to 1440, 1461 to 1480, 1501 to 1520, 1561 to 1580, 1581 to 1600, 1601 to 1620, 1621 to 1640, 1661 to 1680, 1681 to 1700, 1781 to 1800, 1881 to 1900, 1916 to 1935, 1981 to 2000, 2021 to 2040, 2061 to 2080, 2073 to 2092, 2081 to 2100, 2101 to 2120, 2121 to 2140, 2141 to 2160, 2153 to 2172, 2161 to 2180, 2181 to 2200, 2195 to 2214, 2201 to 2220, 2221 to 2240, 2241 to 2260, 2261 to 2280, 2275 to 2294, 2281 to 2300, 2321 to 2340, 2361 to 2380, 2381 to 2400, 2401 to 2420, 2448 to 2467, 2461 to 2480, 2481 to 2500, 2501 to 2520, 2521 to 2540, 2561 to 2580, 2581 to 2600, 2601 to 2620, 2641 to 2660, 2661 to 2680, 2681 to 2700, 2941 to 2960, 3021 to 3040, 3121 to 3140, and 3201 to 3220 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176;
(ii) a base sequence having addition, deletion, or substitution of one or several bases in a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176,
e.g., the base sequence (i).

In one embodiment, the antisense oligonucleotide of the present invention is selected from those having a high ratio of suppressing the TDP-43 gene expression level, measured by the method described in Example 3 of the present specification, obtained in administration to A204 cell. For example, it may be selected from those having a ratio (average value), to the TDP-43 gene expression level obtained in administration of ANT-10 as an antisense oligonucleotide used for comparison, of 1.63 or less (here, “average value” means an average value of a result obtained by using a primer set for detecting exon 2-3 (RNA expression level (exon 2-3)), and a result obtained by using a primer set for detecting exon 5-6 (RNA expression level (exon 5-6))). Examples of such antisense oligonucleotides include antisense oligonucleotides complementary to a nucleic acid comprising at least 15, for example, at least 16, 17, 18, 19, or 20 consecutive bases in a target region selected from the group consisting of positions 239 to 262, 359 to 402, 419 to 462, 519 to 542, 559 to 602, 699 to 722, 819 to 842, 879 to 902, 919 to 982, 1091 to 1122, 1139 to 1162, 1179 to 1202, 1239 to 1262, 1279 to 1342, 1359 to 1382, 1459 to 1482, 1499 to 1522, 1559 to 1602, 1659 to 1682, 1779 to 1802, 1879 to 1902, 1979 to 2002, 2019 to 2042, 2059 to 2122, 2139 to 2216, 2219 to 2242, 2259 to 2302, 2446 to 2469, 2479 to 2502, and 2659 to 2682 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 241 to 260, 361 to 400, 421 to 460, 521 to 540, 561 to 600, 701 to 720, 821 to 840, 881 to 900, 921 to 980, 1093 to 1120, 1141 to 1160, 1181 to 1200, 1241 to 1260, 1281 to 1340, 1361 to 1380, 1461 to 1480, 1501 to 1520, 1561 to 1600, 1661 to 1680, 1781 to 1800, 1881 to 1900, 1981 to 2000, 2021 to 2040, 2061 to 2120, 2141 to 2214, 2221 to 2240, 2261 to 2300, 2448 to 2467, 2481 to 2500, and 2661 to 2680 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 241 to 260, 361 to 380, 381 to 400, 421 to 440, 441 to 460, 521 to 540, 561 to 580, 574 to 593, 581 to 600, 701 to 720, 821 to 840, 881 to 900, 921 to 940, 941 to 960, 961 to 980, 1093 to 1112, 1101 to 1120, 1141 to 1160, 1181 to 1200, 1241 to 1260, 1281 to 1300, 1301 to 1320, 1321 to 1340, 1361 to 1380, 1461 to 1480, 1501 to 1520, 1561 to 1580, 1581 to 1600, 1661 to 1680, 1781 to 1800, 1881 to 1900, 1981 to 2000, 2021 to 2040, 2061 to 2080, 2073 to 2092, 2081 to 2100, 2101 to 2120, 2141 to 2160, 2153 to 2172, 2161 to 2180, 2181 to 2200, 2195 to 2214, 2221 to 2240, 2261 to 2280, 2275 to 2294, 2281 to 2300, 2448 to 2467, 2481 to 2500, and 2661 to 2680 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142;
(ii) a base sequence having addition, deletion, or substitution of one or several bases in a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142,
e.g., the base sequence (i).

In one embodiment, the antisense oligonucleotide of the present invention is complementary to a nucleic acid comprising at least 15, for example, at least 16, 17, 18, 19, or 20 consecutive bases in a target region selected from the group consisting of positions 194 to 217, 350 to 452, 500 to 612, 629 to 684, 689 to 732, 821 to 844, 874 to 972, 1089 to 1177, 1277 to 1338, 1349 to 1392, 1444 to 1537, 1544 to 1592, 1644 to 1697, 1769 to 1812, 1969 to 2012, 2051 to 2317, and 2469 to 2512 from the 5′ end of the base sequence of SEQ ID NO: 1.

In one embodiment, from the antisense oligonucleotide of the present invention, an antisense oligonucleotide or a pharmaceutically acceptable salt thereof, or a hydrate thereof having a sequence consisting of a base sequence selected from the group consisting of SEQ ID NOs: 436 to 438, 440 to 449, 451 to 455, and 472 to 474 is preferably excluded,
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is preferably excluded, and/or
an antisense oligonucleotide having a sequence consisting of a base sequence of SEQ ID NO: 453 or a pharmaceutically acceptable salt thereof, or a hydrate thereof, in which positions 1 to 2, 4, 6, 8 to 9, 11 to 12, and 16 to 20 from the 5′ end of the base sequence of SEQ ID NO: 453 are ENA: 2′-O,4′-C-ethylene bridged nucleic acid, and positions 3, 5, 7, 10, and 13 to 15 are ribonucleotides comprising a 2′-OMe group, is preferably excluded. Besides, in one embodiment, an unmodified antisense oligonucleotide or pharmaceutically acceptable salt thereof, or a hydrate thereof having the base sequence of SEQ ID NO: 450 or SEQ ID NO: 453 is preferably excluded.

In one embodiment, the antisense oligonucleotide of the present invention is complementary to a nucleic acid comprising at least 15, for example, at least 16, 17, 18, 19, or 20 consecutive bases in a target region selected from the group consisting of positions 196 to 215, 352 to 374, 376 to 413, 416 to 450, 502 to 610, 631 to 650, 653 to 682, 691 to 730, 823 to 842, 876 to 970, 1091 to 1175, 1279 to 1336, 1351 to 1390, 1446 to 1535, 1546 to 1590, 1646 to 1695, 1771 to 1810, 1971 to 2010, 2053 to 2250, 2252 to 2315, and 2471 to 2510 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 196 to 215, 352 to 371, 353 to 372, 354 to 373, 355 to 374, 371 to 390, 376 to 395, 386 to 405, 391 to 410, 394 to 413, 406 to 425, 411 to 430, 416 to 435, 426 to 445, 431 to 450, 502 to 521, 506 to 525, 511 to 530, 516 to 535, 517 to 536, 518 to 537, 519 to 538, 520 to 539, 522 to 541, 523 to 542, 524 to 543, 525 to 544, 526 to 545, 527 to 546, 528 to 547, 529 to 548, 531 to 550, 551 to 570, 556 to 575, 566 to 585, 571 to 590, 577 to 596, 586 to 605, 591 to 610, 631 to 650, 653 to 672, 658 to 677, 663 to 682, 691 to 710, 696 to 715, 706 to 725, 711 to 730, 823 to 842, 876 to 895, 877 to 896, 878 to 897, 879 to 898, 880 to 899, 882 to 901, 883 to 902, 884 to 903, 885 to 904, 886 to 905, 891 to 910, 911 to 930, 926 to 945, 927 to 946, 928 to 947, 929 to 948, 931 to 950, 936 to 955, 951 to 970, 1091 to 1110, 1111 to 1130, 1126 to 1145, 1131 to 1150, 1136 to 1155, 1146 to 1165, 1151 to 1170, 1156 to 1175, 1279 to 1298, 1296 to 1315, 1306 to 1325, 1312 to 1331, 1317 to 1336, 1351 to 1370, 1356 to 1375, 1366 to 1385, 1371 to 1390, 1446 to 1465, 1451 to 1470, 1454 to 1473, 1463 to 1482, 1471 to 1490, 1491 to 1510, 1496 to 1515, 1506 to 1525, 1511 to 1530, 1516 to 1535, 1546 to 1565, 1551 to 1570, 1556 to 1575, 1566 to 1585, 1571 to 1590, 1646 to 1665, 1647 to 1666, 1648 to 1667, 1649 to 1668, 1650 to 1669, 1651 to 1670, 1652 to 1671, 1653 to 1672, 1654 to 1673, 1656 to 1675, 1666 to 1685, 1671 to 1690, 1676 to 1695, 1771 to 1790, 1776 to 1795, 1786 to 1805, 1791 to 1810, 1971 to 1990, 1976 to 1995, 1986 to 2005, 1991 to 2010, 2053 to 2072, 2054 to 2073, 2055 to 2074, 2056 to 2075, 2059 to 2078, 2060 to 2079, 2062 to 2081, 2063 to 2082, 2065 to 2084, 2070 to 2089, 2071 to 2090, 2072 to 2091, 2074 to 2093, 2075 to 2094, 2076 to 2095, 2077 to 2096, 2078 to 2097, 2080 to 2099, 2082 to 2101, 2084 to 2103, 2085 to 2104, 2086 to 2105, 2091 to 2110, 2092 to 2111, 2096 to 2115, 2106 to 2125, 2111 to 2130, 2131 to 2150, 2133 to 2152, 2134 to 2153, 2135 to 2154, 2136 to 2155, 2137 to 2156, 2138 to 2157, 2139 to 2158, 2140 to 2159, 2142 to 2161, 2143 to 2162, 2144 to 2163, 2145 to 2164, 2146 to 2165, 2147 to 2166, 2148 to 2167, 2149 to 2168, 2150 to 2169, 2151 to 2170, 2152 to 2171, 2154 to 2173, 2155 to 2174, 2158 to 2177, 2159 to 2178, 2160 to 2179, 2162 to 2181, 2167 to 2186, 2168 to 2187, 2171 to 2190, 2176 to 2195, 2177 to 2196, 2179 to 2198, 2186 to 2205, 2189 to 2208, 2190 to 2209, 2191 to 2210, 2192 to 2211, 2194 to 2213, 2211 to 2230, 2219 to 2238, 2231 to 2250, 2252 to 2271, 2266 to 2285, 2268 to 2287, 2269 to 2288, 2270 to 2289, 2271 to 2290, 2272 to 2291, 2273 to 2292, 2274 to 2293, 2278 to 2297, 2279 to 2298, 2280 to 2299, 2282 to 2301, 2283 to 2302, 2284 to 2303, 2286 to 2305, 2291 to 2310, 2296 to 2315, 2471 to 2490, 2476 to 2495, 2477 to 2496, 2478 to 2497, 2479 to 2498, 2480 to 2499, 2482 to 2501, 2483 to 2502, 2484 to 2503, 2486 to 2505, and 2491 to 2510 from the 5′ end of the base sequence of SEQ ID NO: 1.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 231 to 419, 424 to 425, and 428 to 431, 433 to 435, and 475 to 502;
(ii) a base sequence having addition, deletion, or substitution of one or several bases in a base sequence selected from the group consisting of SEQ ID NOs: 231 to 419, 424 to 425, 428 to 431, 433 to 435, and 475 to 502; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 231 to 419, 424 to 425, 428 to 431, 433 to 435, and 475 to 502,
e.g., the base sequence (i).

In one embodiment, the antisense oligonucleotide of the present invention is selected from those having a high ratio of suppressing the TDP-43 gene expression level, measured by a method described in Example 5 of the present specification, obtained in administration to A204 cell. For example, it may be selected from those having a ratio (average value), to the TDP-43 gene expression level obtained in administration of ANT-10 as an antisense oligonucleotide used for comparison, of 2.56 or less (here, “average value” means an average value of a result obtained by using a primer set for detecting exon 2-3 (RNA expression level (exon 2-3)), and a result obtained by using a primer set for detecting 3′ UTR (RNA expression level (3′ UTR))). Examples of such antisense oligonucleotides include antisense oligonucleotides complementary to a nucleic acid comprising at least 15, for example, at least 16, 17, 18, 19, or 20 consecutive bases in a target region selected from the group consisting of positions 194 to 217, 350 to 452, 500 to 612, 629 to 679, 689 to 732, 821 to 844, 874 to 912, 924 to 972, 1089 to 1177, 1277 to 1338, 1349 to 1392, 1449 to 1484, 1504 to 1532, 1544 to 1592, 1644 to 1697, 1769 to 1812, 1969 to 2012, 2051 to 2252, 2264 to 2317, and 2476 to 2512 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 196 to 215, 352 to 450, 502 to 610, 631 to 650, 653 to 677, 691 to 730, 823 to 842, 876 to 910, 926 to 970, 1091 to 1175, 1279 to 1336, 1351 to 1390, 1451 to 1482, 1506 to 1530, 1546 to 1590, 1646 to 1695, 1771 to 1810, 1971 to 2010, 2053 to 2250, 2266 to 2315, and 2478 to 2510 from the 5′ end of the base sequence of SEQ ID NO: 1.

In one embodiment, the antisense oligonucleotide of the present invention is complementary to a nucleic acid comprising at least 15 consecutive bases in a target region selected from the group consisting of positions 196 to 215, 352 to 371, 353 to 372, 354 to 373, 355 to 374, 371 to 390, 376 to 395, 386 to 405, 391 to 410, 394 to 413, 411 to 430, 416 to 435, 426 to 445, 431 to 450, 502 to 521, 511 to 530, 517 to 536, 518 to 537, 519 to 538, 520 to 539, 522 to 541, 523 to 542, 524 to 543, 525 to 544, 526 to 545, 527 to 546, 528 to 547, 529 to 548, 531 to 550, 551 to 570, 556 to 575, 566 to 585, 571 to 590, 577 to 596, 586 to 605, 591 to 610, 631 to 650, 653 to 672, 658 to 677, 691 to 710, 696 to 715, 711 to 730, 823 to 842, 876 to 895, 877 to 896, 878 to 897, 879 to 898, 880 to 899, 882 to 901, 883 to 902, 884 to 903, 885 to 904, 886 to 905, 891 to 910, 926 to 945, 927 to 946, 928 to 947, 929 to 948, 931 to 950, 951 to 970, 1091 to 1110, 1111 to 1130, 1126 to 1145, 1131 to 1150, 1136 to 1155, 1146 to 1165, 1151 to 1170, 1156 to 1175, 1279 to 1298, 1296 to 1315, 1306 to 1325, 1312 to 1331, 1317 to 1336, 1351 to 1370, 1366 to 1385, 1371 to 1390, 1451 to 1470, 1454 to 1473, 1463 to 1482, 1506 to 1525, 1511 to 1530, 1546 to 1565, 1551 to 1570, 1556 to 1575, 1566 to 1585, 1571 to 1590, 1646 to 1665, 1647 to 1666, 1648 to 1667, 1649 to 1668, 1650 to 1669, 1651 to 1670, 1652 to 1671, 1653 to 1672, 1654 to 1673, 1656 to 1675, 1666 to 1685, 1671 to 1690, 1676 to 1695, 1771 to 1790, 1776 to 1795, 1786 to 1805, 1791 to 1810, 1971 to 1990, 1976 to 1995, 1986 to 2005, 1991 to 2010, 2053 to 2072, 2054 to 2073, 2055 to 2074, 2056 to 2075, 2059 to 2078, 2060 to 2079, 2062 to 2081, 2063 to 2082, 2065 to 2084, 2070 to 2089, 2071 to 2090, 2072 to 2091, 2074 to 2093, 2075 to 2094, 2076 to 2095, 2077 to 2096, 2078 to 2097, 2080 to 2099, 2082 to 2101, 2084 to 2103, 2085 to 2104, 2086 to 2105, 2091 to 2110, 2092 to 2111, 2096 to 2115, 2106 to 2125, 2111 to 2130, 2131 to 2150, 2133 to 2152, 2134 to 2153, 2135 to 2154, 2136 to 2155, 2137 to 2156, 2138 to 2157, 2139 to 2158, 2140 to 2159, 2142 to 2161, 2143 to 2162, 2144 to 2163, 2145 to 2164, 2146 to 2165, 2147 to 2166, 2148 to 2167, 2149 to 2168, 2150 to 2169, 2151 to 2170, 2152 to 2171, 2154 to 2173, 2155 to 2174, 2158 to 2177, 2159 to 2178, 2160 to 2179, 2162 to 2181, 2167 to 2186, 2168 to 2187, 2171 to 2190, 2176 to 2195, 2177 to 2196, 2179 to 2198, 2186 to 2205, 2189 to 2208, 2190 to 2209, 2191 to 2210, 2192 to 2211, 2194 to 2213, 2211 to 2230, 2219 to 2238, 2231 to 2250, 2266 to 2285, 2268 to 2287, 2269 to 2288, 2270 to 2289, 2271 to 2290, 2272 to 2291, 2273 to 2292, 2274 to 2293, 2278 to 2297, 2279 to 2298, 2280 to 2299, 2282 to 2301, 2283 to 2302, 2284 to 2303, 2286 to 2305, 2291 to 2310, 2296 to 2315, 2478 to 2497, 2479 to 2498, 2480 to 2499, 2482 to 2501, 2483 to 2502, 2484 to 2503, and 2491 to 2510 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502;
(ii) a base sequence having addition, deletion, or substitution of one or several bases in a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502,
e.g., the base sequence (i).

In one embodiment, the antisense oligonucleotide of the present invention is selected from those having a high ratio of suppressing the TDP-43 gene expression level, measured by the method described in Example 5 of the present specification, obtained in administration to A204 cell. For example, it may be selected from those having a ratio (average value), to the TDP-43 gene expression level obtained in administration of ANT-10 as an antisense oligonucleotide used for comparison, of 2.05 or less (here, “average value” means an average value of a result obtained by using a primer set for detecting exon 2-3 (RNA expression level (exon 2-3)), and a result obtained by using a primer set for detecting 3′ UTR (RNA expression level (3′ UTR))). Examples of such antisense oligonucleotides include antisense oligonucleotides complementary to the nucleic acid comprising at least 15, for example, at least 16, 17, 18, 19, or 20 consecutive bases in a target region selected from the group consisting of positions 351 to 376, 384 to 452, 509 to 552, 569 to 607, 656 to 679, 821 to 844, 874 to 912, 927 to 952, 1109 to 1177, 1277 to 1338, 1364 to 1392, 1449 to 1484, 1504 to 1532, 1544 to 1587, 1646 to 1697, 1774 to 1812, 1984 to 2007, 2054 to 2127, 2131 to 2215, 2229 to 2252, 2264 to 2312, and 2478 to 2512 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 353 to 374, 386 to 450, 511 to 550, 571 to 605, 658 to 677, 823 to 842, 876 to 910, 929 to 950, 1111 to 1175, 1279 to 1336, 1366 to 1390, 1451 to 1482, 1506 to 1530, 1546 to 1585, 1648 to 1695, 1776 to 1810, 1986 to 2005, 2056 to 2125, 2133 to 2213, 2231 to 2250, 2266 to 2310, and 2480 to 2510 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 353 to 372, 354 to 373, 355 to 374, 386 to 405, 391 to 410, 394 to 413, 411 to 430, 426 to 445, 431 to 450, 511 to 530, 518 to 537, 519 to 538, 520 to 539, 522 to 541, 523 to 542, 524 to 543, 525 to 544, 526 to 545, 527 to 546, 531 to 550, 571 to 590, 577 to 596, 586 to 605, 658 to 677, 823 to 842, 876 to 895, 877 to 896, 878 to 897, 879 to 898, 880 to 899, 882 to 901, 883 to 902, 884 to 903, 885 to 904, 886 to 905, 891 to 910, 929 to 948, 931 to 950, 1111 to 1130, 1126 to 1145, 1131 to 1150, 1146 to 1165, 1151 to 1170, 1156 to 1175, 1279 to 1298, 1296 to 1315, 1306 to 1325, 1312 to 1331, 1317 to 1336, 1366 to 1385, 1371 to 1390, 1451 to 1470, 1463 to 1482, 1506 to 1525, 1511 to 1530, 1546 to 1565, 1551 to 1570, 1566 to 1585, 1648 to 1667, 1649 to 1668, 1650 to 1669, 1651 to 1670, 1652 to 1671, 1653 to 1672, 1654 to 1673, 1666 to 1685, 1671 to 1690, 1676 to 1695, 1776 to 1795, 1786 to 1805, 1791 to 1810, 1986 to 2005, 2056 to 2075, 2059 to 2078, 2060 to 2079, 2062 to 2081, 2063 to 2082, 2065 to 2084, 2070 to 2089, 2071 to 2090, 2072 to 2091, 2074 to 2093, 2075 to 2094, 2076 to 2095, 2077 to 2096, 2080 to 2099, 2082 to 2101, 2084 to 2103, 2085 to 2104, 2086 to 2105, 2091 to 2110, 2092 to 2111, 2096 to 2115, 2106 to 2125, 2133 to 2152, 2134 to 2153, 2135 to 2154, 2136 to 2155, 2137 to 2156, 2138 to 2157, 2139 to 2158, 2142 to 2161, 2143 to 2162, 2144 to 2163, 2145 to 2164, 2146 to 2165, 2147 to 2166, 2148 to 2167, 2149 to 2168, 2150 to 2169, 2151 to 2170, 2152 to 2171, 2154 to 2173, 2155 to 2174, 2158 to 2177, 2159 to 2178, 2160 to 2179, 2162 to 2181, 2171 to 2190, 2176 to 2195, 2177 to 2196, 2186 to 2205, 2189 to 2208, 2190 to 2209, 2191 to 2210, 2192 to 2211, 2194 to 2213, 2231 to 2250, 2266 to 2285, 2268 to 2287, 2269 to 2288, 2270 to 2289, 2271 to 2290, 2272 to 2291, 2273 to 2292, 2274 to 2293, 2280 to 2299, 2282 to 2301, 2283 to 2302, 2284 to 2303, 2286 to 2305, 2291 to 2310, 2480 to 2499, 2482 to 2501, and 2491 to 2510 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502;
(ii) a base sequence having addition, deletion, or substitution of one or several bases in a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502,
e.g., the base sequence (i).

In one embodiment, the antisense oligonucleotide of the present invention is selected from those having a high ratio of suppressing the TDP-43 gene expression level, measured by the method described in Example 5 of the present specification, obtained in administration to A204 cell. For example, it may be selected from those having a ratio (average value), to the TDP-43 gene expression level obtained in administration of ANT-10 as an antisense oligonucleotide used for comparison, of 1.63 or less (here, “average value” means an average value of a result obtained by using a primer set for detecting exon 2-3 (RNA expression level (exon 2-3)), and a result obtained by using a primer set for detecting 3′ UTR (RNA expression level (3′ UTR))). Examples of such antisense oligonucleotides include antisense oligonucleotides complementary to the nucleic acid comprising at least 15, for example, at least 16, 17, 18, 19, or 20 consecutive bases in a target region selected from the group consisting of positions 409 to 452, 509 to 548, 656 to 679, 874 to 907, 927 to 950, 1129 to 1172, 1294 to 1333, 1449 to 1484, 1504 to 1532, 1549 to 1587, 1646 to 1697, 1784 to 1807, 2057 to 2117, 2131 to 2215, 2266 to 2312, and 2478 to 2501 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 411 to 450, 511 to 546, 658 to 677, 876 to 905, 929 to 948, 1131 to 1170, 1296 to 1331, 1451 to 1482, 1506 to 1530, 1551 to 1585, 1648 to 1695, 1786 to 1805, 2059 to 2115, 2133 to 2213, 2268 to 2310, and 2480 to 2499 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 411 to 430, 426 to 445, 431 to 450, 511 to 530, 520 to 539, 522 to 541, 523 to 542, 524 to 543, 526 to 545, 527 to 546, 658 to 677, 876 to 895, 878 to 897, 879 to 898, 880 to 899, 882 to 901, 883 to 902, 886 to 905, 929 to 948, 1131 to 1150, 1146 to 1165, 1151 to 1170, 1296 to 1315, 1312 to 1331, 1451 to 1470, 1463 to 1482, 1506 to 1525, 1511 to 1530, 1551 to 1570, 1566 to 1585, 1648 to 1667, 1649 to 1668, 1650 to 1669, 1651 to 1670, 1652 to 1671, 1653 to 1672, 1654 to 1673, 1671 to 1690, 1676 to 1695, 1786 to 1805, 2059 to 2078, 2060 to 2079, 2062 to 2081, 2070 to 2089, 2071 to 2090, 2072 to 2091, 2074 to 2093, 2075 to 2094, 2076 to 2095, 2082 to 2101, 2084 to 2103, 2085 to 2104, 2086 to 2105, 2091 to 2110, 2092 to 2111, 2096 to 2115, 2133 to 2152, 2134 to 2153, 2135 to 2154, 2136 to 2155, 2137 to 2156, 2138 to 2157, 2139 to 2158, 2142 to 2161, 2143 to 2162, 2144 to 2163, 2145 to 2164, 2146 to 2165, 2148 to 2167, 2149 to 2168, 2150 to 2169, 2151 to 2170, 2152 to 2171, 2159 to 2178, 2160 to 2179, 2162 to 2181, 2176 to 2195, 2177 to 2196, 2186 to 2205, 2191 to 2210, 2192 to 2211, 2194 to 2213, 2268 to 2287, 2269 to 2288, 2271 to 2290, 2272 to 2291, 2273 to 2292, 2274 to 2293, 2280 to 2299, 2282 to 2301, 2283 to 2302, 2284 to 2303, and 2291 to 2310, and 2480 to 2499 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502;
(ii) a base sequence having addition, deletion, or substitution of one or several bases in a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 414, 478, 482, 487, 489, 490, 493, 495, and 502; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502,
e.g., the base sequence (i).

In one embodiment, the antisense oligonucleotide of the present invention is selected from those having a high ratio of suppressing the TDP-43 gene expression level, measured by the method described in Example 5 of the present specification, obtained in administration to A204 cell. For example, it may be selected from those having a ratio (average value), to the TDP-43 gene expression level obtained in administration of ANT-10 as an antisense oligonucleotide used for comparison, of 1.20 or less (here, “average value” means an average value of a result obtained by using a primer set for detecting exon 2-3 (RNA expression level (exon 2-3)), and a result obtained by using a primer set for detecting 3′ UTR (RNA expression level (3′ UTR))). Examples of such antisense oligonucleotides include antisense oligonucleotides complementary to the nucleic acid comprising at least 15, for example, at least 16, 17, 18, 19, or 20 consecutive bases in a target region selected from the group consisting of positions 520 to 548, 880 to 903, 927 to 950, 1129 to 1172, 1461 to 1484, 1509 to 1532, 1549 to 1572, 1647 to 1697, 1784 to 1807, 2069 to 2117, 2131 to 2215, and 2269 to 2305 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 522 to 546, 882 to 901, 929 to 948, 1131 to 1170, 1463 to 1482, 1511 to 1530, 1551 to 1570, 1649 to 1671, 1676 to 1695, 1786 to 1805, 2071 to 2115, 2133 to 2178, 2162 to 2213, and 2271 to 2303 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 522 to 541, 523 to 542, 526 to 545, 527 to 546, 882 to 901, 929 to 948, 1131 to 1150, 1146 to 1165, 1151 to 1170, 1463 to 1482, 1511 to 1530, 1551 to 1570, 1649 to 1668, 1650 to 1669, 1651 to 1670, 1652 to 1671, 1676 to 1695, 1786 to 1805, 2071 to 2090, 2072 to 2091, 2074 to 2093, 2082 to 2101, 2086 to 2105, 2096 to 2115, 2133 to 2152, 2134 to 2153, 2135 to 2154, 2136 to 2155, 2139 to 2158, 2142 to 2161, 2143 to 2162, 2144 to 2163, 2145 to 2164, 2148 to 2167, 2152 to 2171, 2159 to 2178, 2162 to 2181, 2176 to 2195, 2177 to 2196, 2186 to 2205, 2194 to 2213, 2271 to 2290, 2272 to 2291, 2273 to 2292, 2274 to 2293, 2280 to 2299, 2282 to 2301, and 2284 to 2303 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490;
(ii) a base sequence having addition, deletion, or substitution of one or several bases in a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490,
e.g., the base sequence (i).

In one embodiment, the antisense oligonucleotide of the present invention is complementary to a nucleic acid comprising at least 15, for example, at least 16, 17, 18, 19, or 20 consecutive bases in a target region selected from the group consisting of positions 519 to 547, 879 to 902, 1459 to 1482, 1559 to 1582, 1647 to 1672, 1674 to 1697, 2059 to 2107, 2132 to 2215, 2269 to 2301, and 2479 to 2502 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 521 to 545, 881 to 900, 1461 to 1480, 1561 to 1580, 1649 to 1670, 1676 to 1695, 2061 to 2105, 2134 to 2213, 2271 to 2299, and 2481 to 2500 from the 5′ end of the base sequence of SEQ ID NO: 1.

In one embodiment, the antisense oligonucleotide of the present invention is complementary to a nucleic acid comprising at least 15, for example, at least 16, 17, 18, 19, or 20 consecutive bases in a target region selected from the group consisting of positions 521 to 540, 522 to 541, 523 to 542, 526 to 545, 881 to 900, 1461 to 1480, 1561 to 1580, 1649 to 1668, 1650 to 1669, 1651 to 1670, 1676 to 1695, 2061 to 2080, 2073 to 2092, 2074 to 2093, 2081 to 2100, 2086 to 2105, 2134 to 2153, 2135 to 2154, 2136 to 2155, 2144 to 2163, 2145 to 2164, 2152 to 2171, 2161 to 2180, 2176 to 2195, 2194 to 2213, 2271 to 2290, 2273 to 2292, 2274 to 2293, 2275 to 2294, 2280 to 2299, and 2481 to 2500 from the 5′ end of the base sequence of SEQ ID NO: 1.

An example of the antisense oligonucleotide complementary to the nucleic acid comprising at least 15 consecutive bases in the target sequence includes an antisense oligonucleotide comprising or consisting of:
(i) a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402;
(ii) a base sequence having addition, deletion, or substitution of one or several bases in a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402; or
(iii) a base sequence having 75% or more, 80% or more, 85% or more, and preferably 90% or more, 95% or more, 98% or more, or 99% or more sequence identity with a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402,
e.g., the base sequence (i).

In one embodiment, the antisense oligonucleotide of the present invention reduces TDP-43 gene expression level (e.g., RNA expression level of an mRNA or a pre-mRNA that is a transcriptional product). It can be tested in a manner described in Examples of the present specification whether or not the antisense oligonucleotide of the present invention reduces TDP-43 gene expression level. For example, it can be determined whether or not TDP-43 gene expression level is reduced by introducing the antisense oligonucleotide of the present invention into a cell expressing TDP-43 gene, such as A204 cell, and examining whether or not the expression level of the TDP-43 gene expressed in the cell is lower (by, for example, 5% or more, 10% or more, or 20% or more) as compared with that in a negative control (e.g., a sample containing only a medium).

The antisense oligonucleotide of the present invention can be easily synthesized using various automated synthesizers (e.g., AKTA oligopilot plus 10/100 (GE Healthcare), or automated nucleic acid synthesizer NTS M-8-MX DNA/RNA or NTS H-6 DNA/RNA). Alternatively, the synthesis can also be entrusted to a third-party organization (e.g., Promega Corp. or Takara Co.).

In one embodiment, the present invention relates to a pharmaceutical composition comprising an antisense oligonucleotide or a pharmaceutically acceptable salt thereof, or a hydrate thereof. When the antisense oligonucleotide of the present invention is to be administered to a subject, the pharmaceutical composition of the present invention may comprise a carrier to promote delivery of the antisense oligonucleotide. Such a carrier is not particularly limited as far as it is pharmaceutically acceptable, and examples include cationic carriers such as cationic liposomes, and cationic polymers, and carriers using viral envelope. The cationic liposomes include, for example, liposomes composed of 2-O-(2-diethylaminoethyl)carabamoyl-1,3-O-dioleoylglycerol and phospholipids as the essential constituents (hereinafter referred to as “liposome A”), Oligofectamine (registered trademark) (manufactured by Invitrogen Corp.), Lipofectin (registered trademark) (manufactured by Invitrogen Corp.), Lipofectamine (registered trademark) (manufactured by Invitrogen Corp.), Lipofectamine 2000 (registered trademark) (manufactured by Invitrogen Corp.), DMRIE-C (registered trademark) (manufactured by Invitrogen Corp.), GeneSilencer (registered trademark) (manufactured by Gene Therapy Systems), TransMessenger (registered trademark) (manufactured by QIAGEN, Inc.), TransIT TKO (registered trademark) (manufactured by Mirus), and Nucleofector II (Lonza). Examples of the cationic polymers are JetSI (registered trademark) (manufactured by Qbiogene, Inc.), and Jet-PEI (registered trademark) (polyethylenimine, manufactured by Qbiogene, Inc.). An example of carriers using viral envelop is GenomeOne (registered trademark) (HVJ-E liposome, manufactured by Ishihara Sangyo). Alternatively, the medical devices described in Japanese Patent No. 2924179, and the cationic carriers described in Japanese Domestic Re-Publication of PCT Application Nos. 2006/129594 and 2008/096690 may be used as well.

In one embodiment, the antisense oligonucleotide of the present invention may be in the form of a complex (conjugate) with a lipid or the like in the pharmaceutical composition for promoting delivery of the antisense oligonucleotide. For example, as described in Bijsterbosch, M. K. et al., Nucleic Acids Research, 2000, vol. 28, pp. 2717-2725, the antisense oligonucleotide may be in the form of a conjugate with cholesterol. Besides, to the antisense oligonucleotide according to one embodiment, a cell-penetrating peptide (CPP) consisting of a cationic amino acid, or comprising a large amount of a cationic amino acid may be bound at one of or both of the 5′ end and the 3′ end. The CPP is not especially limited, and may be, for example, a polypeptide Rn (wherein n is a natural number of 2 to 8) comprising n number of consecutive arginine R residues.

The pharmaceutical composition of the present invention may comprise pharmaceutically acceptable additives in addition to the antisense oligonucleotide or the pharmaceutically acceptable salt thereof, or the hydrate thereof and optionally the carrier described above. Examples of such additives are emulsification aids (e.g., fatty acids having 6 to 22 carbon atoms and their pharmaceutically acceptable salts, albumin and dextran), stabilizers (e.g., cholesterol, phosphatidic acid, mannitol, and sorbitol), isotonizing agents (e.g., sodium chloride, glucose, maltose, lactose, sucrose, and trehalose), and pH adjusters (e.g., hydrochloric acid, sulfuric acid, phosphoric acid, acetic acid, sodium hydroxide, potassium hydroxide, and triethanolamine). One or more of these additives can be used. The content of the additive in the composition of the present invention is appropriately 90 wt % or less, preferably 70 wt % or less, and more preferably 50 wt % or less.

The preparation method of the pharmaceutical composition of the present invention is not limited, and the preparation may be conducted by, for example, adding the antisense oligonucleotide of the present invention to a dispersion of the carrier, and appropriately stirring the resultant. The additive may be added in an appropriate step either before or after the addition of the antisense oligonucleotide of the present invention. An aqueous solvent used in adding the antisense oligonucleotide of the present invention is not particularly limited as long as it is pharmaceutically acceptable, and examples include injectable water, injectable distilled water, an electrolyte fluid such as physiological saline, and a sugar solution such as a glucose solution, or a maltose solution. Those skilled in the art can appropriately choose conditions for pH and temperature to be employed in this case.

The pharmaceutical composition of the present invention may be prepared into, for example, a liquid form or its lyophilized preparation. The lyophilized preparation can be prepared by lyophilizing the composition of the present invention in a liquid form in a conventional manner. The lyophilization can be performed, for example, by appropriately sterilizing the composition of the present invention in a liquid form, dispensing an aliquot into a vial container, performing preliminary freezing for 2 hours at conditions in a range of about −40° C. to −20° C., performing a primary drying in a range of about 0° C. to 10° C. under reduced pressure, and then performing a secondary drying in a range of about 15° C. to 25° C. under reduced pressure. In addition, in general, the lyophilized preparation of the composition of the present invention can be obtained by replacing the content of the vial with nitrogen gas and capping the resultant.

The lyophilized preparation of the pharmaceutical composition of the present invention can be used in general upon reconstitution by adding an optional suitable solution (reconstitution liquid). Examples of such a reconstitution liquid include injectable water, physiological saline and other general infusion fluids. A volume of the reconstitution liquid may vary depending on the intended use, etc., is not particularly limited, and is suitably 0.5-fold to 2-fold greater than the volume prior to the lyophilization or no more than 500 mL.

It is desired to control a dose of the pharmaceutical composition of the present invention to be administered by taking the following factors into account: the type and dosage form of the antisense oligonucleotide of the present invention contained; patients' conditions including age, body weight, etc.; administration route; and the characteristics and extent of the disease. A single dose for an adult calculated as the amount of the antisense oligonucleotide of the present invention can be 0.01 mg to 20 mg per kg body weight, preferably 0.03 mg to 10 mg per kg body weight, more preferably 0.05 mg to 4 mg per kg body weight, and further preferably 0.1 mg to 2 mg per kg body weight. The frequency of administration may be once per 1 to 3 days, once per week, or once per 2 to 3 weeks. This numerical range may vary occasionally depending on the type of the target disease, the administration route and the target molecule. Therefore, a dose or frequency of administration lower than these ranges may be sufficient in some occasion and conversely, a dose or frequency of administration higher than these ranges may be required occasionally.

The administration form for the pharmaceutical composition of the present invention is not particularly limited as long as it is pharmaceutically acceptable route for administration, and can be chosen depending upon method of treatment. Examples include intravenous administration, intraarterial administration, intramuscular administration, subcutaneous administration, oral administration, tissue administration, transdermal administration, pulmonary administration, nasal administration, and administration to central nerve. Examples of the administration to central nerve include intrathecal administration, intracranial administration, e.g., intracerebroventricular administration or lateral ventricle administration, intraparenchymal administration, and administration to leptomeninges (pia mater). Also, dosage forms which are available for the composition of the present invention are not particularly limited, and include, for example, various injections, oral agents, drips, inhalations, ointments, lotions, and poultices.

The subject to be given the antisense oligonucleotide or the pharmaceutical composition of the present invention includes e.g. mammals, including primates such as humans, experimental animals such as rats, mice, and brown rats, and domestic animals such as pigs, cows, horses, and sheep, and the subject is preferably a human.

In one embodiment, the present invention relates to an antisense oligonucleotide or a pharmaceutically acceptable salt thereof, or a hydrate thereof, or a pharmaceutical composition of the present invention for treating and/or preventing a TDP-43 proteinopathy.

In one embodiment, the present invention relates to a method for treating and/or preventing a TDP-proteinopathy, comprising a step of administering, to a subject, the antisense oligonucleotide or the pharmaceutically acceptable salt thereof, or the hydrate thereof, or the pharmaceutical composition of the present invention. The pharmaceutical composition, and the dose, the administration route and the like thereof in the present embodiment are the same as those described herein.

In one embodiment, the present invention relates to use of an antisense oligonucleotide or a pharmaceutically acceptable salt thereof, or a hydrate thereof, or a pharmaceutical composition of the present invention of the present invention in production of a medicament for use in a method for treating and/or preventing a TDP-43 proteinopathy.

As used herein, the term “TDP-43 proteinopathy” means a neurodegenerative disease related to accumulation of TDP-43. Examples of the TDP-43 proteinopathy include amyotrophic lateral sclerosis (ALS), frontotemporal lobar degeneration (FTLD), Perry syndrome, and Lewy body disease, and amyotrophic lateral sclerosis is preferred.

As used herein, treatment of a TDP-43 proteinopathy encompasses one or more of relief, improvement, and remission of the TDP-43 proteinopathy or symptoms thereof. As used herein, prevention of a TDP-43 proteinopathy encompasses reduction of risks of causing the TDP-43 proteinopathy or symptoms thereof.

### EXAMPLES

Hereinafter, the present invention will be described in more detail with reference to Examples and Test Examples below, but the present invention is not limited thereto.

The term “gapmer” is defined as an oligomer compound, an oligonucleotide in general, having a center region mainly constituted by a deoxyoligonucleotide, and sandwiched between two adjacent segments of non-deoxyoligonucleotides. The center region is called “gap”, and the adjacent segments are called “wings”. The gapmer used in the present examples has a gap of 10 nucleotides sandwiched between two adjacent wings of 5 nucleotides. This is called a 5-10-5 gapmer.

### Example 1: Synthesis of Gapmer (Oligonucleotide)

In the gapmers used in the present example, some specific oligonucleotides having, in a nucleoside of the wing, a 2′-methoxyethyl (2′-O-MOE) group represented by the following formula (a) were synthesized referring to the method described in W. Brad Wan et al., Nucleic Acids Research, 2014, Vol. 42, No. 22, pp. 13456-13468.

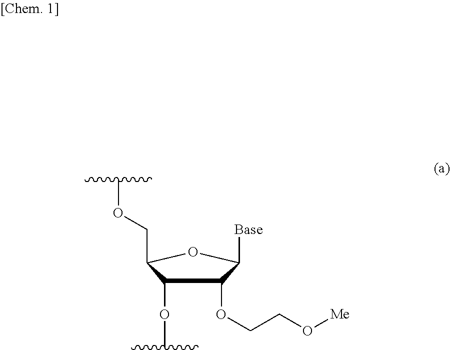

wherein Base represents 5-methylcytosine (C), thymine (T), adenine (A), or guanine (G), and Me represents methyl.

A 20 mer gapmer (oligonucleotide) to be tested having, in a nucleoside of the wing, the 2′-O-MOE group represented by the formula (a) was synthesized at 0.5, 1, or 10 μmol scale with an automated nucleic acid synthesizer, NTS M-8-MX DNA/RNA (NIHON TECHNO SERVICE CO., LTD.), or an automated nucleic acid synthesizer, NTS H-6 DNA/RNA (NHON TECHNO SERVICE CO., LTD.). The extension of chain length was performed in accordance with standard phosphoramidite protocol (solid-phase carrier is Glen UnySupport, regarding sulfurization, DDTT ([(N,N-Dimethylaminomethylidene)amino]-3H-1,2,4-dithiazoline-3-thione) or the like was used). Thus, an oligonucleotide in which a hydroxyl group at the 5′-position at the end was protected with a dimethoxytrityl (DMTr) group, and the 3′-position was carried on a solid-phase was obtained. Subsequently, after removing the DMTr group by treatment with deblocking solution-1 obtained from FUJIFILM Wako Pure Chemical Corporation, the resultant was subjected to ammonia treatment, and thus, an objective product was cut out from the solid-phase carrier. The solvent was distilled off, and the thus obtained crude product was purified by reverse phase column chromatography with Sep-Pak C18 Plus Short Cartridge, 360 mg Sorbent per Cartridge, 55-105 μm, 50/pk (Waters), or YMC_Triart_C18. In some cases, purification by anion exchange, and desalt by reverse phase chromatography were employed together. A fraction comprising an objective product was collected and concentrated under reduced pressure, and the thus obtained residue was dissolved in water, and freeze dried to give the objective product in the form of a white flocculent solid. The objective product was obtained as a triethylamine salt or a sodium salt. The molecular weight of the obtained objective product was checked by ESI-TOF-MS.

### Example 2: Design of Gapmer (Oligonucleotide) to be Tested

A gapmer (oligonucleotide) to be tested was designed as an antisense oligonucleotide (ASO) targeting an mRNA of human TDP-43 (Gen Bank: NM_007375.4, SEQ ID NO: 1) or a pre-mRNA thereof. Sequences of oligonucleotides expressed by DNA bases are shown in Table 2. Here, “Start site in SEQ ID NO: 1” indicates the number (position), from the 5′ end of the base sequence of SEQ ID NO: 1, of the base at the 5′ end of the target region of each ASO. Also, “End site in SEQ ID NO: 1” indicates the number (position), from the 5′ end of the base sequence of SEQ ID NO: 1, of the base at the 3′ end of the target region of each ASO.

In Table 2, all oligonucleotides are 5-10-5 gapmers, wing regions thereof are all ribonucleotides having 2′-O-MOE modification, gap regions are all deoxyribonucleotides, and bonds between nucleosides are all phosphorothioates (P═S). Besides, in the gapmers of Table 2, C (cytosine) in the gap region and the wing regions is actually methyl C, and T in the wing regions is actually T.

TABLE 2
Gapmer (oligonucleotide) to be tested having 2′-O-MOE group in wing nucleoside

| | Start site | End site | | | |
|---|---|---|---|---|---|
| ASO base sequence (DNA) | in SEQ ID | in SEQ ID | Target | ASO | SEQ ID |
| 5′ to 3′ | NO: 1 | NO: 1 | exon | name | NO: |
| CCGCTTCGCTCCCACAAAAT | 1 | 20 | 5′UTR | ANT-22 | 2 |
| ACCCAAGCGCAGCCCAGCCA | 21 | 40 | 5′UTR | ANT-23 | 3 |
| GGACACCGAAGCAGCGACGG | 41 | 60 | 5′UTR | ANT-24 | 4 |
| GCTGCTGGGAAGCCCGACAG | 61 | 80 | 5′UTR | ANT-25 | 5 |
| TTTACTTTTCCCGCTAGGCC | 81 | 100 | 5′UTR | ANT-26 | 6 |
| CACCGTCCCATCGTCTTCCG | 161 | 180 | 2 | ANT-30 | 7 |
| GCTGTAACCGTGGAGAGCAG | 181 | 200 | 2 | ANT-31 | 8 |
| CACACGCCCCTGGAAACTGG | 201 | 220 | 2 | ANT-32 | 9 |
| TGGATTCCTGTAGCGAAGCC | 221 | 240 | 2 | ANT-33 | 10 |
| CCTCTCATACACTGAGACAC | 241 | 260 | 2 | ANT-34 | 11 |
| TTCCTTCTACCAGCCGGACA | 261 | 280 | 2 | ANT-35 | 12 |
| AGCATCTGGGGCATGCAGAA | 281 | 300 | 2 | ANT-36 | 13 |
| TACACCAGATTTCCCCAGCC | 301 | 320 | 2 | ANT-38 | 14 |
| CTTTTGGATAGTTGACAACA | 321 | 340 | 2 | ANT-39 | 15 |
| ATCCATTTTTCTTTTGTTAT | 341 | 360 | 3 | ANT-40 | 16 |
| GCTGATGAAGCATCTGTCTC | 361 | 380 | 3 | ANT-41 | 17 |
| CTGCTCTTTTCACTTTCACT | 381 | 400 | 3 | ANT-42 | 18 |
| TAAATCGGATGTTTTCTGGA | 401 | 420 | 3 | ANT-43 | 19 |
| CATGGGAGACCCAACACTAT | 421 | 440 | 3 | ANT-44 | 20 |
| GGTCCTGTTCGGTTGTTTTC | 441 | 460 | 3 | ANT-45 | 21 |
| GGTACTAAAATACTCTTTCA | 461 | 480 | 3 | ANT-46 | 22 |
| ACCATAAGAACTTCTCCAAA | 481 | 500 | 3 | ANT-47 | 23 |
| TAAGATCTTTCTTGACCTGC | 501 | 520 | 3 | ANT-48 | 24 |
| CCCCTTTGAATGACCAGTCT | 521 | 540 | 4 | ANT-49 | 25 |
| GTAAAACGAACAAAGCCAAA | 541 | 560 | 4 | ANT-50 | 26 |
| TCACTTGTGTTTCATATTCC | 561 | 580 | 4 | ANT-51 | 27 |
| TGTGACATTACTTTCACTTG | 574 | 593 | 4 | ANT-37 | 28 |
| ATGTCGCTGTGACATTACTT | 581 | 600 | 4 | ANT-52 | 29 |
| CACCATCGTCCATCTATCAT | 601 | 620 | 4 | ANT-53 | 30 |
| AATTAGGAAGTTTGCAGTCA | 621 | 640 | 4 | ANT-54 | 31 |
| CTCATCTTGGCTTTGCTTAG | 641 | 660 | 4 | ANT-55 | 32 |
| ACTTTTCTGCTTCTCAAAGG | 661 | 680 | 5 | ANT-56 | 33 |
| CTGTACAGCGCCCCACAAAC | 681 | 700 | 5 | ANT-57 | 34 |
| CTCATCCTCAGTCATGTCCT | 701 | 720 | 5 | ANT-58 | 35 |
| TGAGAGAAGAACTCCCGCAG | 721 | 740 | 5 | ANT-59 | 36 |
| CATCCATCACATCCCCGTAC | 741 | 760 | 5 | ANT-60 | 37 |
| GAATGGCTTGGGGATGAAGA | 761 | 780 | 5 | ANT-61 | 38 |
| GTAACAAAGGCAAAGGCCCT | 781 | 800 | 5 | ANT-62 | 39 |
| CAATCTGATCATCTGCAAAT | 801 | 820 | 5 | ANT-63 | 40 |
| CTCTCCACAAAGAGACTGCG | 821 | 840 | 6 | ANT-64 | 41 |
| GCTATTGTGCTTAGGTTCGG | 881 | 900 | 6 | ANT-67 | 42 |
| CTTCTTTCTAACTGTCTATT | 901 | 920 | 6 | ANT-68 | 43 |
| GATTACCACCAAATCTTCCA | 921 | 940 | 6 | ANT-69 | 44 |
| CAAAGCCACCTGGATTACCA | 933 | 952 | 6 | ANT-1 | 45 |
| CTGATTCCCAAAGCCACCTG | 941 | 960 | 6 | ANT-70 | 46 |
| CTGCTATTACCAAATCCACC | 961 | 980 | 6 | ANT-71 | 47 |
| CCAAACCAGCTCCACCCCCT | 981 | 1000 | 6 | ANT-72 | 48 |
| ATTACTACCTTGATTGTTTC | 1001 | 1020 | 6 | ANT-73 | 49 |
| AAGTTCATCCCACCACCCAT | 1021 | 1040 | 6 | ANT-74 | 50 |
| GATTAATGCTGAACGCACCA | 1041 | 1060 | 6 | ANT-75 | 51 |
| GGCGGCAGCCATCATGGCTG | 1061 | 1080 | 6 | ANT-76 | 52 |
| CTGCTCTGTAGTGCTGCCTG | 1081 | 1100 | 6 | ANT-77 | 53 |
| ATCATACCCCAACTGCTCTG | 1093 | 1112 | 6 | ANT-2 | 54 |
| ACATGCCCATCATACCCCAA | 1101 | 1120 | 6 | ANT-78 | 55 |
| CTGGTTCTGCTGGCTGGCTA | 1121 | 1140 | 6 | ANT-79 | 56 |
| TTATTACCCGATGGGCCTGA | 1141 | 1160 | 6 | ANT-80 | 57 |
| GCATGTTGCCTTGGTTTTGG | 1161 | 1180 | 6 | ANT-81 | 58 |
| GGCCTGGTTTGGCTCCCTCT | 1181 | 1200 | 6 | ANT-82 | 59 |
| GAGTTATTTCCAGAACCGAA | 1201 | 1220 | 6 | ANT-83 | 60 |
| CAGAATTAGAGCCACTATAA | 1221 | 1240 | 6 | ANT-84 | 61 |
| TCCCCAACCAATTGCTGCAC | 1241 | 1260 | 6 | ANT-85 | 62 |
| GACCCTGCATTGGATGCTGA | 1261 | 1280 | 6 | ANT-86 | 63 |
| CTCCATTAAAACCACTGCCC | 1281 | 1300 | 6 | ANT-87 | 64 |
| ATCCATGCTTGAGCCAAAGC | 1301 | 1320 | 6 | ANT-88 | 65 |
| CCCCAGCCAGAAGACTTAGA | 1321 | 1340 | 6 | ANT-89 | 66 |
| ACAACCCCACTGTCTACATT | 1341 | 1360 | 6 | ANT-90 | 67 |
| CCATTCTATACCAACCAACC | 1361 | 1380 | 3′UTR | ANT-91 | 68 |
| TAGAAAAATTTGAATTCCCA | 1381 | 1400 | 3′UTR | ANT-92 | 69 |
| CTTACCATGAGTTTAGAAAA | 1394 | 1413 | 3′UTR | ANT-3 | 70 |
| CAATATACTTACCATGAGTT | 1401 | 1420 | 3′UTR | ANT-93 | 71 |
| CTTAGTACATATGTATTTTA | 1421 | 1440 | 3′UTR | ANT-94 | 72 |
| ACAAACCAATTTTGAAAATT | 1441 | 1460 | 3′UTR | ANT-95 | 73 |
| CTGAATATACTCCACACTGA | 1461 | 1480 | 3′UTR | ANT-96 | 74 |
| ATGTCAAAAATACTGCTGAA | 1476 | 1495 | 3′UTR | ANT-4 | 75 |
| GAAAAATGTCAAAAATACTG | 1481 | 1500 | 3′UTR | ANT-97 | 76 |
| TAGCTCTTCCTTTTTCTAAA | 1501 | 1520 | 3′UTR | ANT-98 | 77 |
| CAAAACTTATAAAATTCCTT | 1521 | 1540 | 3′UTR | ANT-99 | 78 |
| TATTTCAACCTTTCATGTAA | 1541 | 1560 | 3′UTR | ANT-100 | 79 |
| GTTCACTTTCAACCACTCAA | 1561 | 1580 | 3′UTR | ANT-101 | 80 |
| TACCAATCAGGCAAACAGCA | 1581 | 1600 | 3′UTR | ANT-102 | 81 |
| TCAATTGTAGTGTGTTGGTT | 1601 | 1620 | 3′UTR | ANT-103 | 82 |
| ACAGGAGAAACCTTTTGATA | 1621 | 1640 | 3′UTR | ANT-104 | 83 |
| AAGTCCAGGGATAAAATATT | 1641 | 1660 | 3′UTR | ANT-105 | 84 |
| ATGCAAAGAATTCACTTGAC | 1661 | 1680 | 3′UTR | ANT-106 | 85 |
| CAATGGTTTCCGTTTTGAAC | 1681 | 1700 | 3′UTR | ANT-107 | 86 |
| GTAAAGAATGTAGTTCTAAT | 1701 | 1720 | 3′UTR | ANT-108 | 87 |
| GGTTCAAATTAAAACAAGGG | 1721 | 1740 | 3′UTR | ANT-109 | 88 |
| GAAAAAAATCCATATGGTGG | 1741 | 1760 | 3′UTR | ANT-110 | 89 |
| TAAAAGGAGATTTTCTTAAG | 1761 | 1780 | 3′UTR | ANT-111 | 90 |
| ACTGTGACACCATGATCTCC | 1781 | 1800 | 3′UTR | ANT-112 | 91 |
| ACAAAACAAAAGAACCAAAC | 1801 | 1820 | 3′UTR | ANT-113 | 92 |
| ATGAGATGAACTGCAGAGAT | 1876 | 1895 | 3′UTR | ANT-5 | 93 |
| TTGAAATGAGATGAACTGCA | 1881 | 1900 | 3′UTR | ANT-117 | 94 |
| AGTGCTTCTTCCATAAACAT | 1901 | 1920 | 3′UTR | ANT-118 | 95 |
| CACTACTTTCAATGAAGTGC | 1916 | 1935 | 3′UTR | ANT-6 | 96 |
| TACAGCACTACTTTCAATGA | 1921 | 1940 | 3′UTR | ANT-119 | 97 |
| TATTCCTATGGCAGAATATT | 1941 | 1960 | 3′UTR | ANT-120 | 98 |
| ATGAGAAAGCATGTAGACAG | 1961 | 1980 | 3′UTR | ANT-121 | 99 |
| GCGTGATGACGAATTCTTGA | 1981 | 2000 | 3′UTR | ANT-122 | 100 |
| TCAAAGACGCGGCCTGTGAT | 2001 | 2020 | 3′UTR | ANT-123 | 101 |
| TAAAAATGGGACACCCACCG | 2021 | 2040 | 3′UTR | ANT-124 | 102 |
| AAGAGTAGCGGATAAAAATG | 2033 | 2052 | 3′UTR | ANT-7 | 103 |
| ATGAAATAAAGAGTAGCGGA | 2041 | 2060 | 3′UTR | ANT-125 | 104 |
| CATAGCGTTGATACGACTCC | 2061 | 2080 | 3′UTR | ANT-126 | 105 |
| CAGCCTTGCGTTCATAGCGT | 2073 | 2092 | 3′UTR | ANT-8 | 106 |
| CCATATCACAGCCTTGCGTT | 2081 | 2100 | 3′UTR | ANT-127 | 107 |
| GTTCAGACAGCCTTCTGGTT | 2101 | 2120 | 3′UTR | ANT-128 | 108 |
| TCCCACACAAGGTTTCAAAA | 2121 | 2140 | 3′UTR | ANT-129 | 109 |
| TGCCTCGGCACCACCATCAA | 2141 | 2160 | 3′UTR | ANT-130 | 110 |
| ACTAGCCTTTCATGCCTCGG | 2153 | 2172 | 3′UTR | ANT-9 | 111 |
| TCGCTCATACTAGCCTTTCA | 2161 | 2180 | 3′UTR | ANT-131 | 112 |
| GCACGCGCTCTCTCCTTTTC | 2181 | 2200 | 3′UTR | ANT-132 | 113 |
| ACCACCAAGTCTCTGCACGC | 2195 | 2214 | 3′UTR | ANT-10 | 114 |
| TTATGCACCACCAAGTCTCT | 2201 | 2220 | 3′UTR | ANT-133 | 115 |
| GCCAAGTTAAAAAATATCCA | 2221 | 2240 | 3′UTR | ANT-134 | 116 |
| AGGATTGAGAGACACATCTC | 2241 | 2260 | 3′UTR | ANT-135 | 117 |
| CACTCTCTCACCAAAGCCAC | 2261 | 2280 | 3′UTR | ANT-136 | 118 |
| CATTGCTCTCTGCACACTCT | 2275 | 2294 | 3′UTR | ANT-11 | 119 |
| TGCTATCATTGCTCTCTGCA | 2281 | 2300 | 3′UTR | ANT-137 | 120 |
| AAAAACATTCGTACATTATT | 2301 | 2320 | 3′UTR | ANT-138 | 121 |
| GTGGATGTCCTTTGAATGCA | 2321 | 2340 | 3′UTR | ANT-139 | 122 |
| TTAAAAGTCTTCCAACAGAT | 2341 | 2360 | 3′UTR | ANT-140 | 123 |
| ATCTAAGAACAAAAACTCAC | 2361 | 2380 | 3′UTR | ANT-141 | 124 |
| ACATTCATCTAATGTGGGTT | 2381 | 2400 | 3′UTR | ANT-142 | 125 |
| CAAGTATCATTTCACTTAAC | 2401 | 2420 | 3′UTR | ANT-143 | 126 |
| ACAAAGGGGTAGGGGGAGTA | 2421 | 2440 | 3′UTR | ANT-144 | 127 |
| TACAGCATTCACAGCAGTTG | 2441 | 2460 | 3′UTR | ANT-145 | 128 |
| CACACCATACAGCATTCACA | 2448 | 2467 | 3′UTR | ANT-12 | 129 |
| ACAGAAGAGAACACACACCA | 2461 | 2480 | 3′UTR | ANT-146 | 130 |
| GCCACACTTACATATCAGTA | 2481 | 2500 | 3′UTR | ANT-147 | 131 |
| CATCAGCTTCAGTTCACATT | 2501 | 2520 | 3′UTR | ANT-148 | 132 |
| CTCAGTCCATGTTCTCAGCC | 2521 | 2540 | 3′UTR | ANT-149 | 133 |
| CCACAAGCTCAGTCCATGTT | 2528 | 2547 | 3′UTR | ANT-13 | 134 |
| CCTGCAAAGCACACCACAAG | 2541 | 2560 | 3′UTR | ANT-150 | 135 |
| TGAACTCTGCTTCAAGTCCT | 2561 | 2580 | 3′UTR | ANT-151 | 136 |
| GAGACACCTGAGCTCACTGG | 2581 | 2600 | 3′UTR | ANT-152 | 137 |
| TAGAACTTCCACCCTTCTTT | 2601 | 2620 | 3′UTR | ANT-153 | 138 |
| TATGGGTAGCTAACAGACAT | 2621 | 2640 | 3′UTR | ANT-154 | 139 |
| ACTGCAGCAAACAGCATTCT | 2641 | 2660 | 3′UTR | ANT-155 | 140 |
| CACAGAACTGCAGCAAACAG | 2647 | 2666 | 3′UTR | ANT-14 | 141 |
| ATCCAAGCACAGGACACAGA | 2661 | 2680 | 3′UTR | ANT-156 | 142 |
| TGACAACTCTTATAAAAAGC | 2681 | 2700 | 3′UTR | ANT-157 | 143 |
| AACAATGACAACTCTTATAA | 2686 | 2705 | 3′UTR | ANT-15 | 144 |
| TATTTAAGAATTTCCAACAA | 2701 | 2720 | 3′UTR | ANT-158 | 145 |
| CATATTATTTAAATCAGTTT | 2721 | 2740 | 3′UTR | ANT-159 | 146 |
| AAAACAAAGACACATATTAT | 2733 | 2752 | 3′UTR | ANT-16 | 147 |
| AGGGCTGCAAAACAAAGACA | 2741 | 2760 | 3′UTR | ANT-160 | 148 |
| GCTATGAATTCTTTGCATTC | 2761 | 2780 | 3′UTR | ANT-161 | 149 |
| TAACTGCTATGAATTCTTTG | 2766 | 2785 | 3′UTR | ANT-17 | 150 |
| TCAAAAAAGGGGAATTAACT | 2781 | 2800 | 3′UTR | ANT-162 | 151 |
| AAAGTTCCATCTCAAAAGGG | 2801 | 2820 | 3′UTR | ANT-163 | 152 |
| TTTATGAAAGTTCCATCTCA | 2807 | 2826 | 3′UTR | ANT-18 | 153 |
| TACTGCCAAGAAACTTTATG | 2821 | 2840 | 3′UTR | ANT-164 | 154 |
| TTATTTGAAGCAAAATAAAC | 2841 | 2860 | 3′UTR | ANT-165 | 155 |
| ATAAGTTTATTTGAAGCAAA | 2847 | 2866 | 3′UTR | ANT-19 | 156 |
| AGACAACTTTTCAAATAAGT | 2861 | 2880 | 3′UTR | ANT-166 | 157 |
| TGATGAATCCATTTGACTTG | 2881 | 2900 | 3′UTR | ANT-167 | 158 |
| GACAGGTGATGAATCCATTT | 2887 | 2906 | 3′UTR | ANT-20 | 159 |
| AGGTGTCAATGCATGACAGG | 2901 | 2920 | 3′UTR | ANT-168 | 160 |
| ACCAATTAAGTCTGGGTATC | 2921 | 2940 | 3′UTR | ANT-169 | 161 |
| TGGCCAATGCAAGAACAAAT | 2941 | 2960 | 3′UTR | ANT-170 | 162 |
| AAAAAAAAAAATTTTCACTT | 2961 | 2980 | 3′UTR | ANT-171 | 163 |
| TCAAAACTAGATTTCAAAAG | 2981 | 3000 | 3′UTR | ANT-172 | 164 |
| GTGCGGTCACCCAGACTTAT | 3001 | 3020 | 3′UTR | ANT-173 | 165 |
| TTAGGTGCGGTCACCCAGAC | 3005 | 3024 | 3′UTR | ANT-21 | 166 |
| GGTACTGCTTACCATTTTAG | 3021 | 3040 | 3′UTR | ANT-174 | 167 |
| GCACTAAGAAAAAGCCGGAG | 3041 | 3060 | 3′UTR | ANT-175 | 168 |
| CATCACCCAAATGCACAGAG | 3061 | 3080 | 3′UTR | ANT-176 | 169 |
| CACAGGCCATGTAAATAGAA | 3081 | 3100 | 3′UTR | ANT-177 | 170 |
| GACTTCCCAATGGAGATTTA | 3101 | 3120 | 3′UTR | ANT-178 | 171 |
| AGAATCTTTTTAGAAGGCAT | 3121 | 3140 | 3′UTR | ANT-179 | 172 |
| TTTGCCCACTCCCCCAAATA | 3141 | 3160 | 3′UTR | ANT-180 | 173 |
| CATTAGAAAATAATCAACAT | 3161 | 3180 | 3′UTR | ANT-181 | 174 |
| TGATATGCTTTGCTACAAAG | 3181 | 3200 | 3′UTR | ANT-182 | 175 |
| GCTGATATTCCCTTTTCAAT | 3201 | 3220 | 3′UTR | ANT-183 | 176 |
| AAATCCCAAACTAGGAAGGT | 3221 | 3240 | 3′UTR | ANT-184 | 177 |
| TGCAATTAATTCCACTTTTC | 3241 | 3260 | 3′UTR | ANT-185 | 178 |
| TTCTTCTACTTTATCCCTAC | 3261 | 3280 | 3′UTR | ANT-186 | 179 |
| GGCACAAGATAATTTGTGGT | 3281 | 3300 | 3′UTR | ANT-187 | 180 |
| AGGCCTCTTAATGGATTTCA | 3301 | 3320 | 3′UTR | ANT-188 | 181 |
| CCCTAATTCTTAAAGCTATC | 3321 | 3340 | 3′UTR | ANT-189 | 182 |
| ACTTCCAGACAGACAACCCA | 3341 | 3360 | 3′UTR | ANT-190 | 183 |
| CAAAGCCCATTCCACTTAAC | 3361 | 3380 | 3′UTR | ANT-191 | 184 |
| TTCCCCCACCTCCTGGAGGA | 3381 | 3400 | 3′UTR | ANT-192 | 185 |
| CTGTATTCAATGTTACCACA | 3401 | 3420 | 3′UTR | ANT-193 | 186 |
| TTGTAAGCGATTTTATTCAA | 3421 | 3440 | 3′UTR | ANT-194 | 187 |
| GCATTGTGAGAGTGTGAGTT | 3441 | 3460 | 3′UTR | ANT-195 | 188 |
| GCTTTTACATACTTAACAAT | 3461 | 3480 | 3′UTR | ANT-196 | 189 |
| AACAGAGAATCAATGTTATT | 3481 | 3500 | 3′UTR | ANT-197 | 190 |
| AATTAGTTACAAAAAAGTAC | 3501 | 3520 | 3′UTR | ANT-198 | 191 |
| AATGAGCTCAACTCTCACAG | 3521 | 3540 | 3′UTR | ANT-199 | 192 |
| TCACATTCTTCCAACTAGAA | 3541 | 3560 | 3′UTR | ANT-200 | 193 |
| AACTACCAACACAACAAATA | 3561 | 3580 | 3′UTR | ANT-201 | 194 |
| TTAGGTAAGGGCATTAGGTA | 3581 | 3600 | 3′UTR | ANT-202 | 195 |
| ACCTATTTATCATAATCTAA | 3601 | 3620 | 3′UTR | ANT-203 | 196 |
| TGTAACTTGCAAAATGACAA | 3621 | 3640 | 3′UTR | ANT-204 | 197 |
| ACTTCATTGATAAATGTTTA | 3641 | 3660 | 3′UTR | ANT-205 | 198 |
| GATTACAAGTCTAAAGGATG | 3661 | 3680 | 3′UTR | ANT-206 | 199 |
| GAATAATGAAACAATGTGGC | 3681 | 3700 | 3′UTR | ANT-207 | 200 |
| ATCCCTTTACAGAGGAAACT | 3701 | 3720 | 3′UTR | ANT-208 | 201 |
| AAAAATTAAAACAACTCAAG | 3721 | 3740 | 3′UTR | ANT-209 | 202 |
| CAGATTCAGATGCAGAAAAA | 3741 | 3760 | 3′UTR | ANT-210 | 203 |
| TACAGGGTTTGGAAATCATG | 3761 | 3780 | 3′UTR | ANT-211 | 204 |
| AAAATGCAAAATTCAGATGG | 3781 | 3800 | 3′UTR | ANT-212 | 205 |
| TGAGTAATAGTGCAAGTGCT | 3801 | 3820 | 3′UTR | ANT-213 | 206 |
| TGTTACCATGTTACTGCTGC | 3821 | 3840 | 3′UTR | ANT-214 | 207 |
| TCCCCGAGTACCATTTTAAG | 3841 | 3860 | 3′UTR | ANT-215 | 208 |
| GTCAGTTTAGTCTTTGGAGG | 3861 | 3880 | 3′UTR | ANT-216 | 209 |
| CCTGGGCTCCTTGAAGGCTT | 3881 | 3900 | 3′UTR | ANT-217 | 210 |
| CGTTGACAAGTTAACTTACC | 3901 | 3920 | 3′UTR | ANT-218 | 211 |
| AAGAAGGGATTAAACCATGC | 3921 | 3940 | 3′UTR | ANT-219 | 212 |
| CTGAAATTTACACAAGTGTA | 3941 | 3960 | 3′UTR | ANT-220 | 213 |
| AAGCCTTCTATGACCAGTAA | 3961 | 3980 | 3′UTR | ANT-221 | 214 |
| AAAAGGCCACTCAACATTGA | 3981 | 4000 | 3′UTR | ANT-222 | 215 |
| AGTACCATAAACATGTTAAT | 4001 | 4020 | 3′UTR | ANT-223 | 216 |
| ATAAATACCCGTATCTATGC | 4021 | 4040 | 3′UTR | ANT-224 | 217 |
| CAAAATCTTCTTAGGGTAAA | 4041 | 4060 | 3′UTR | ANT-225 | 218 |
| GTTTAAGTACTTTTAAACTT | 4061 | 4080 | 3′UTR | ANT-226 | 219 |
| AAACAAATCTTTGCCAAATA | 4081 | 4100 | 3′UTR | ANT-227 | 220 |
| TTGACCAAATAGATTTTTAA | 4101 | 4120 | 3′UTR | ANT-228 | 221 |
| TTAGAATGAATGCATTTAGA | 4121 | 4140 | 3′UTR | ANT-229 | 222 |
| TATCTGGTTCAAAAAATTTT | 4141 | 4160 | 3′UTR | ANT-230 | 223 |
| TGTCAAAAAAAAATTTTATT | 4161 | 4180 | 3′UTR | ANT-231 | 224 |

### Example 3: TDP-43 Gene Knockdown Activity Test of Gapmer (Antisense Oligonucleotide)

### Test Example 1: Measurement of Knockdown Activity (Inhibitory Activity) of Human TDP-43 Gene

Into 3.0×10⁵ A204 cells (human rhabdomyosarcoma cell line, obtained from ATCC), each of gapmers shown in Table 2 was introduced in a concentration of 4.5 μM with 4D-Nucleofector™ using SF Cell Line 4D-Nucleofector™ X Kit S in accordance with the protocol attached to the kit. DS-130 was used as the program.

After the introduction, the cells were cultured overnight in 0.5 mL of McCoy's 5A Medium (SIGMA) containing a 10% fetal bovine serum (FBS) (Nichirei Corporation) and 1 mM L-glutamine (Gibco) under conditions of 37° C. and 5% CO₂.

The tested substance had a 2′-O-MOE group in a nucleoside of the wing, and were all 5-10-5 gapmers, and bonds between nucleosides were all phosphorothioates (P═S). Besides, C (cytosine) in the gap region and the wing regions was actually methyl C, and T (thymine) in the wing regions was actually T.

After washing the resultant cells with PBS (Nacalai Tesque, Inc.) once, buffer RA1 (Takara Bio Inc.) containing 1% 2-mercaptoethanol (Nacalai Tesque, Inc.) was added thereto to dissolve the cells, which were collected on NucleoSpin(registered trademark) Filter (Takara Bio Inc.). The resultant filter was centrifuged at 11,000×g for 1 minute to collect a filtered liquid as a homogenate. Total RNA was extracted from the homogenate with NucleoSpin(registered trademark) RNA (Takara Bio Inc.) in accordance with the protocol attached thereto. The concentration of the extracted total RNA was measured with NanoDrop one (Thermo Fisher).

250 ng of the extracted total RNA was subjected to a reverse transcription (RT) reaction with High Capacity cDNA Reverse Transcription Kit (Applied Biosystems) and a random primer attached to the kit. Specifically, in accordance with the protocol attached to the kit, a reaction solution was prepared. TaKaRa PCR Thermal Cycler Dice Touch (Takara Bio Inc.) was used as the thermal cycler. The program of the RT reaction used was as follows.
25° C., 10 minutes: primer annealing
37° C., 120 minutes: reverse transcription reaction
85° C., 5 minutes: reverse transcriptase inactivation

Thereafter, qPCR was performed with Fast SYBR Green Master Mix (Thermo Fisher Scientific) using the RT reaction product as a template, and the TDP-43 and β-actin RNA expression levels endogenously expressed in the A204 cells were measured. Specifically, in accordance with the protocol attached to the kit, a reaction solution was prepared. For the qPCR, QuantStudio 6 Flex Real-Time PCR System (Applied Biosystems) was used. The program of the qPCR used was as follows.
50° C., 2 minutes; and 95° C., 10 minutes: polymerase activation, cDNA thermal denaturation
[95° C., 15 seconds; and 60° C., 1 minute]×40 cycles: PCR Amplification
95° C., 15 seconds; and 60° C., 1 minute to 95° C., 15 seconds (Melting Curve 0.05° C./s): melting curve analysis

Primers used in the detection of the TDP-43 RNA are shown in Table 3. Sequences of 2 portions (exon 2 to exon 3, and exon 5 to exon 6) in TDP-43 were respectively amplified and detected. A primer used in detection of the β-actin RNA of house keeping gene is shown in Table 4.

The TDP-43 RNA detection value was corrected with a β-actin RNA detection value of house keeping gene. Besides, a ratio of the TDP-43 RNA corrected value of cells having been subjected to the introduction operation with each gapmer added thereto to the TDP-43 RNA corrected value of cells having been subjected to the introduction operation only with 4D-Nucleofector™ without adding the gapmer thereto (“vehicle”) was calculated, and the thus obtained ratio was used as the TDP-43 RNA expression level to analyze the inhibitory activity of each gapmer. As the RNA expression level, the result obtained by using a primer set for detecting exon 2-3 (RNA expression level (exon 2-3)), and the result obtained by using a primer set for detecting exon 5-6 (RNA expression level (exon 5-6)) are both shown. Besides, as a gapmer for comparison, ANT-10 (SEQ ID NO: 114) that had been confirmed to have activity through a preliminary test (no data shown) was used. Specifically, based on a ratio of the TDP-43 RNA expression level of cells having been subjected to the introduction operation with each gapmer added thereto to the TDP-43 RNA expression level of cells having been subjected to the introduction operation with the gapmer shown as ANT-10 (SEQ ID NO: 114) added thereto, the RNA expression level ratio to ANT-10 (SEQ ID NO: 114) was calculated. In the calculation, as the TDP-43 RNA expression level of the cells having been subjected to the introduction operation with the gapmer shown as ANT-10 (SEQ ID NO: 114) added thereto, a value obtained in each assay was used. The results are shown in Table 5 ((wherein “Ratio of RNA expression level/ANT-10 (exon 2-3)” indicates the RNA expression level ratio obtained using the primer set for detecting exon 2-3, and “Ratio of RNA expression level/ANT-10 (exon 5-6)” indicates the RNA expression level ratio obtained using the primer set for detecting exon 5-6. Besides, “Average of ratio of RNA expression level” indicates the average of the ratio of RNA expression level/ANT-10 (exon 2-3) and the ratio of RNA expression level/ANT-10 (exon 5-6). As for ANT-10 (SEQ ID NO: 114), representative values obtained through a plurality of times of assay are shown).

TABLE 3
Primer used to detect RNA of Endogenous TDP-43 and recognized exon site

| | Primer set for detection | | | |
|---|---|---|---|---|
| Detected | | SEQ ID | | SEQ ID |
| exon | Sense primer 5′ to 3′ | NO: | Reverse primer 5′ to 3′ | NO: |
| exon 2-3 | GGGAAATCTGGTGTATGTTGTC | 225 | TTTCAGGTCCTGTTCGGTTG | 226 |
| exon 5-6 | GCGCTGTACAGAGGACATGA | 227 | AGTTCATCCCACCACCCATA | 228 |

TABLE 4
Primer used to detect RNA of B-Actin used as gene for correction

| | Primer set for detection | | | |
|---|---|---|---|---|
| Gene for | | SEQ ID | | SEQ ID |
| correction | Sense primer 5′ to 3′ | NO: | Reverse primer 5′ to 3′ | NO: |
| β-actin | GAAGATCAAGATCATTGCTCCT | 229 | TACTCCTGCTTGCTGATCCA | 230 |

TABLE 5
Human TDP-43 gene RNA expression level, etc. in A204 cell treated with respective
gapmers (4.5 μM)

| | | | Ratio of | | Ratio of | |
|---|---|---|---|---|---|---|
| | | | RNA | | RNA | |
| | | RNA | expression | RNA | expression | Average of |
| | | expression | level/ | expression | level/ | ratio of RNA |
| | | level | ANT-10 | level | ANT-10 | expression |
| ASO name | SEQ ID NO: | (exon2-3) | (exon2-3) | (exon5-6) | (exon5-6) | level |
| vehicle | — | 1 | 4.610 | 1 | 5.741 | 5.175 |
| ANT-10 | 114 | 0.217 | 1.000 | 0.174 | 1.000 | 1.000 |
| ANT-22 | 2 | 0.712 | 4.165 | 0.693 | 4.501 | 4.333 |
| ANT-23 | 3 | 0.377 | 2.202 | 0.427 | 2.773 | 2.487 |
| ANT-24 | 4 | 0.760 | 4.441 | 0.789 | 5.124 | 4.783 |
| ANT-25 | 5 | 0.791 | 4.626 | 0.874 | 5.680 | 5.153 |
| ANT-26 | 6 | 0.436 | 2.550 | 0.538 | 3.496 | 3.023 |
| ANT-30 | 7 | 0.402 | 2.352 | 0.513 | 3.333 | 2.842 |
| ANT-31 | 8 | 0.689 | 4.029 | 0.778 | 5.055 | 4.542 |
| ANT-32 | 9 | 0.521 | 3.044 | 0.536 | 3.482 | 3.263 |
| ANT-33 | 10 | 0.463 | 2.705 | 0.580 | 3.769 | 3.237 |
| ANT-34 | 11 | 0.214 | 1.252 | 0.260 | 1.692 | 1.472 |
| ANT-35 | 12 | 0.292 | 1.706 | 0.362 | 2.352 | 2.029 |
| ANT-36 | 13 | 0.726 | 4.388 | 0.784 | 3.721 | 4.054 |
| ANT-38 | 14 | 0.520 | 3.143 | 0.698 | 3.311 | 3.227 |
| ANT-39 | 15 | 0.427 | 2.584 | 0.511 | 2.427 | 2.506 |
| ANT-40 | 16 | 0.524 | 3.170 | 0.659 | 3.128 | 3.149 |
| ANT-41 | 17 | 0.153 | 0.928 | 0.417 | 1.979 | 1.453 |
| ANT-42 | 18 | 0.067 | 0.406 | 0.098 | 0.463 | 0.434 |
| ANT-43 | 19 | 0.323 | 1.950 | 0.389 | 1.845 | 1.898 |
| ANT-44 | 20 | 0.120 | 0.723 | 0.249 | 1.184 | 0.954 |
| ANT-45 | 21 | 0.125 | 0.757 | 0.472 | 2.240 | 1.498 |
| ANT-46 | 22 | 0.347 | 2.099 | 0.607 | 2.883 | 2.491 |
| ANT-47 | 23 | 0.609 | 3.678 | 0.719 | 3.412 | 3.545 |
| ANT-48 | 24 | 0.348 | 2.103 | 0.415 | 1.969 | 2.036 |
| ANT-49 | 25 | 0.033 | 0.200 | 0.053 | 0.253 | 0.227 |
| ANT-50 | 26 | 0.414 | 2.503 | 0.532 | 2.526 | 2.514 |
| ANT-51 | 27 | 0.110 | 0.665 | 0.147 | 0.696 | 0.680 |
| ANT-37 | 28 | 0.211 | 1.186 | 0.247 | 1.577 | 1.382 |
| ANT-52 | 29 | 0.142 | 0.856 | 0.179 | 0.850 | 0.853 |
| ANT-53 | 30 | 0.518 | 3.130 | 0.708 | 3.362 | 3.246 |
| ANT-55 | 32 | 0.441 | 2.667 | 0.501 | 2.378 | 2.522 |
| ANT-56 | 33 | 0.825 | 4.985 | 0.980 | 4.651 | 4.818 |
| ANT-57 | 34 | 0.386 | 2.335 | 0.440 | 2.086 | 2.211 |
| ANT-58 | 35 | 0.178 | 1.074 | 0.035 | 0.167 | 0.620 |
| ANT-59 | 36 | 0.366 | 2.213 | 0.247 | 1.174 | 1.693 |
| ANT-60 | 37 | 0.342 | 2.067 | 0.265 | 1.257 | 1.662 |
| ANT-61 | 38 | 0.815 | 4.928 | 0.893 | 4.240 | 4.584 |
| ANT-62 | 39 | 0.423 | 2.560 | 0.451 | 2.139 | 2.349 |
| ANT-63 | 40 | 0.757 | 4.574 | 0.815 | 3.867 | 4.220 |
| ANT-64 | 41 | 0.479 | 2.898 | 0.064 | 0.303 | 1.601 |
| ANT-67 | 42 | 0.233 | 1.177 | 0.143 | 1.031 | 1.104 |
| ANT-68 | 43 | 0.726 | 3.669 | 0.608 | 4.373 | 4.021 |
| ANT-69 | 44 | 0.199 | 1.005 | 0.132 | 0.952 | 0.978 |
| ANT-1 | 45 | 0.612 | 2.802 | 0.409 | 1.594 | 2.198 |
| ANT-70 | 46 | 0.309 | 1.558 | 0.156 | 1.120 | 1.339 |
| ANT-71 | 47 | 0.284 | 1.435 | 0.228 | 1.637 | 1.536 |
| ANT-72 | 48 | 0.882 | 4.454 | 0.137 | 0.988 | 2.721 |
| ANT-73 | 49 | 0.802 | 4.051 | 0.844 | 6.069 | 5.060 |
| ANT-74 | 50 | 0.858 | 4.335 | 0.273 | 1.963 | 3.149 |
| ANT-75 | 51 | 0.299 | 1.511 | 0.268 | 1.927 | 1.719 |
| ANT-76 | 52 | 0.515 | 2.599 | 0.215 | 1.543 | 2.071 |
| ANT-77 | 53 | 0.358 | 1.808 | 0.214 | 1.540 | 1.674 |
| ANT-2 | 54 | 0.368 | 1.685 | 0.322 | 1.255 | 1.470 |
| ANT-78 | 55 | 0.249 | 1.257 | 0.196 | 1.410 | 1.334 |
| ANT-79 | 56 | 0.544 | 2.750 | 0.373 | 2.683 | 2.716 |
| ANT-80 | 57 | 0.194 | 0.979 | 0.125 | 0.899 | 0.939 |
| ANT-81 | 58 | 0.406 | 2.050 | 0.327 | 2.351 | 2.201 |
| ANT-82 | 59 | 0.313 | 1.579 | 0.227 | 1.633 | 1.606 |
| ANT-83 | 60 | 0.396 | 2.000 | 0.356 | 2.560 | 2.280 |
| ANT-84 | 61 | 0.288 | 1.455 | 0.260 | 1.866 | 1.661 |
| ANT-85 | 62 | 0.270 | 1.363 | 0.216 | 1.550 | 1.457 |
| ANT-86 | 63 | 0.452 | 2.285 | 0.374 | 2.690 | 2.488 |
| ANT-87 | 64 | 0.280 | 1.414 | 0.233 | 1.673 | 1.543 |
| ANT-88 | 65 | 0.215 | 1.087 | 0.191 | 1.371 | 1.229 |
| ANT-89 | 66 | 0.253 | 1.276 | 0.203 | 1.461 | 1.369 |
| ANT-90 | 67 | 0.538 | 2.719 | 0.474 | 3.411 | 3.065 |
| ANT-91 | 68 | 0.098 | 0.493 | 0.051 | 0.368 | 0.430 |
| ANT-92 | 69 | 0.544 | 2.748 | 0.477 | 3.428 | 3.088 |
| ANT-3 | 70 | 1.054 | 4.828 | 1.110 | 4.322 | 4.575 |
| ANT-93 | 71 | 0.872 | 4.404 | 0.725 | 5.216 | 4.810 |
| ANT-94 | 72 | 0.349 | 1.764 | 0.294 | 2.112 | 1.938 |
| ANT-96 | 74 | 0.130 | 0.659 | 0.090 | 0.645 | 0.652 |
| ANT-4 | 75 | 0.785 | 3.597 | 0.875 | 3.408 | 3.503 |
| ANT-98 | 77 | 0.163 | 0.922 | 0.157 | 0.963 | 0.942 |
| ANT-100 | 79 | 0.816 | 4.615 | 0.833 | 5.113 | 4.864 |
| ANT-101 | 80 | 0.076 | 0.429 | 0.065 | 0.397 | 0.413 |
| ANT-102 | 81 | 0.248 | 1.400 | 0.221 | 1.353 | 1.377 |
| ANT-103 | 82 | 0.337 | 1.907 | 0.418 | 2.567 | 2.237 |
| ANT-104 | 83 | 0.376 | 2.125 | 0.415 | 2.546 | 2.336 |
| ANT-105 | 84 | 0.782 | 4.420 | 0.799 | 4.899 | 4.659 |
| ANT-106 | 85 | 0.144 | 0.816 | 0.117 | 0.719 | 0.767 |
| ANT-107 | 86 | 0.297 | 1.678 | 0.259 | 1.590 | 1.634 |
| ANT-109 | 88 | 0.862 | 4.873 | 0.773 | 4.742 | 4.808 |
| ANT-110 | 89 | 0.851 | 4.813 | 0.627 | 3.848 | 4.330 |
| ANT-112 | 91 | 0.112 | 0.631 | 0.077 | 0.471 | 0.551 |
| ANT-5 | 93 | 0.732 | 3.354 | 0.756 | 2.945 | 3.149 |
| ANT-117 | 94 | 0.240 | 1.357 | 0.233 | 1.430 | 1.393 |
| ANT-118 | 95 | 0.876 | 4.951 | 0.850 | 5.215 | 5.083 |
| ANT-6 | 96 | 0.625 | 2.865 | 0.684 | 2.666 | 2.766 |
| ANT-120 | 98 | 0.758 | 4.282 | 0.838 | 5.141 | 4.712 |
| ANT-121 | 99 | 0.612 | 3.458 | 0.595 | 3.648 | 3.553 |
| ANT-122 | 100 | 0.210 | 1.188 | 0.212 | 1.299 | 1.244 |
| ANT-123 | 101 | 0.729 | 4.120 | 0.724 | 4.441 | 4.280 |
| ANT-124 | 102 | 0.354 | 1.998 | 0.196 | 1.204 | 1.601 |
| ANT-125 | 104 | 0.747 | 4.225 | 0.617 | 3.783 | 4.004 |
| ANT-126 | 105 | 0.127 | 0.716 | 0.115 | 0.708 | 0.712 |
| ANT-8 | 106 | 0.157 | 0.725 | 0.109 | 0.624 | 0.675 |
| ANT-127 | 107 | 0.132 | 0.607 | 0.091 | 0.520 | 0.564 |
| ANT-128 | 108 | 0.154 | 0.711 | 0.135 | 0.773 | 0.742 |
| ANT-129 | 109 | 0.389 | 1.795 | 0.326 | 1.869 | 1.832 |
| ANT-130 | 110 | 0.164 | 0.754 | 0.092 | 0.530 | 0.642 |
| ANT-9 | 111 | 0.223 | 1.023 | 0.255 | 0.994 | 1.009 |
| ANT-131 | 112 | 0.087 | 0.399 | 0.056 | 0.324 | 0.362 |
| ANT-132 | 113 | 0.188 | 0.865 | 0.134 | 0.771 | 0.818 |
| ANT-133 | 115 | 0.372 | 1.713 | 0.329 | 1.888 | 1.800 |
| ANT-134 | 116 | 0.340 | 1.569 | 0.283 | 1.622 | 1.596 |
| ANT-135 | 117 | 0.544 | 2.507 | 0.485 | 2.784 | 2.646 |
| ANT-136 | 118 | 0.274 | 1.264 | 0.237 | 1.362 | 1.313 |
| ANT-11 | 119 | 0.179 | 0.822 | 0.195 | 0.761 | 0.792 |
| ANT-137 | 120 | 0.144 | 0.662 | 0.145 | 0.833 | 0.747 |
| ANT-139 | 122 | 0.382 | 1.761 | 0.321 | 1.841 | 1.801 |
| ANT-140 | 123 | 1.030 | 4.747 | 0.901 | 5.174 | 4.961 |
| ANT-141 | 124 | 0.533 | 2.456 | 0.430 | 2.468 | 2.462 |
| ANT-142 | 125 | 0.583 | 2.686 | 0.487 | 2.797 | 2.742 |
| ANT-143 | 126 | 0.521 | 2.404 | 0.443 | 2.545 | 2.474 |
| ANT-144 | 127 | 0.811 | 3.740 | 0.696 | 3.996 | 3.868 |
| ANT-145 | 128 | 0.654 | 3.013 | 0.576 | 3.309 | 3.161 |
| ANT-12 | 129 | 0.174 | 0.976 | 0.156 | 0.998 | 0.987 |
| ANT-146 | 130 | 0.469 | 2.161 | 0.393 | 2.257 | 2.209 |
| ANT-147 | 131 | 0.249 | 1.147 | 0.225 | 1.292 | 1.219 |
| ANT-148 | 132 | 0.554 | 2.555 | 0.462 | 2.654 | 2.604 |
| ANT-149 | 133 | 0.438 | 2.018 | 0.384 | 2.205 | 2.111 |
| ANT-13 | 134 | 0.689 | 3.158 | 0.816 | 3.179 | 3.169 |
| ANT-150 | 135 | 0.651 | 2.999 | 0.554 | 3.180 | 3.089 |
| ANT-151 | 136 | 0.610 | 2.594 | 0.608 | 2.738 | 2.666 |
| ANT-152 | 137 | 0.481 | 2.047 | 0.450 | 2.028 | 2.037 |
| ANT-153 | 138 | 0.384 | 1.630 | 0.399 | 1.797 | 1.713 |
| ANT-154 | 139 | 0.730 | 3.104 | 0.716 | 3.222 | 3.163 |
| ANT-155 | 140 | 0.500 | 2.125 | 0.506 | 2.278 | 2.201 |
| ANT-14 | 141 | 0.743 | 3.403 | 0.802 | 3.124 | 3.263 |
| ANT-156 | 142 | 0.367 | 1.561 | 0.369 | 1.664 | 1.612 |
| ANT-157 | 143 | 0.528 | 2.245 | 0.512 | 2.307 | 2.276 |
| ANT-15 | 144 | 1.057 | 4.840 | 1.224 | 4.766 | 4.803 |
| ANT-158 | 145 | 0.871 | 3.702 | 0.920 | 4.142 | 3.922 |
| ANT-159 | 146 | 0.710 | 3.020 | 0.714 | 3.215 | 3.117 |
| ANT-160 | 148 | 0.873 | 3.713 | 0.901 | 4.057 | 3.885 |
| ANT-161 | 149 | 0.758 | 3.223 | 0.777 | 3.497 | 3.360 |
| ANT-17 | 150 | 0.836 | 4.700 | 0.797 | 5.093 | 4.896 |
| ANT-162 | 151 | 0.913 | 3.882 | 0.944 | 4.252 | 4.067 |
| ANT-163 | 152 | 0.940 | 3.995 | 1.008 | 4.540 | 4.267 |
| ANT-18 | 153 | 0.856 | 4.814 | 0.656 | 4.191 | 4.502 |
| ANT-164 | 154 | 0.753 | 3.199 | 0.874 | 3.934 | 3.567 |
| ANT-165 | 155 | 0.877 | 3.728 | 0.953 | 4.293 | 4.011 |
| ANT-166 | 157 | 0.955 | 4.060 | 0.902 | 4.061 | 4.060 |
| ANT-167 | 158 | 0.828 | 3.521 | 0.863 | 3.884 | 3.703 |
| ANT-168 | 160 | 0.869 | 3.695 | 0.923 | 4.158 | 3.926 |
| ANT-169 | 161 | 0.745 | 3.166 | 0.784 | 3.531 | 3.348 |
| ANT-170 | 162 | 0.600 | 2.549 | 0.743 | 3.347 | 2.948 |
| ANT-171 | 163 | 0.962 | 4.090 | 0.919 | 4.136 | 4.113 |
| ANT-172 | 164 | 0.791 | 3.363 | 0.876 | 3.946 | 3.654 |
| ANT-173 | 165 | 0.880 | 3.740 | 0.873 | 3.932 | 3.836 |
| ANT-21 | 166 | 0.777 | 4.366 | 0.865 | 5.527 | 4.946 |
| ANT-174 | 167 | 0.590 | 2.506 | 0.586 | 2.640 | 2.573 |
| ANT-175 | 168 | 0.869 | 3.693 | 0.924 | 4.159 | 3.926 |
| ANT-176 | 169 | 0.734 | 3.120 | 0.762 | 3.431 | 3.276 |
| ANT-177 | 170 | 0.766 | 3.254 | 0.749 | 3.371 | 3.313 |
| ANT-178 | 171 | 0.676 | 2.876 | 0.717 | 3.228 | 3.052 |
| ANT-179 | 172 | 0.601 | 2.554 | 0.666 | 2.999 | 2.776 |
| ANT-180 | 173 | 0.965 | 4.103 | 1.083 | 4.877 | 4.490 |
| ANT-183 | 176 | 0.551 | 3.170 | 0.507 | 2.759 | 2.964 |
| ANT-184 | 177 | 0.769 | 4.420 | 0.813 | 4.424 | 4.422 |
| ANT-187 | 180 | 0.872 | 5.016 | 0.970 | 5.281 | 5.148 |
| ANT-188 | 181 | 0.661 | 3.798 | 0.699 | 3.807 | 3.802 |
| ANT-189 | 182 | 0.845 | 4.857 | 0.839 | 4.569 | 4.713 |
| ANT-190 | 183 | 0.674 | 3.877 | 0.770 | 4.191 | 4.034 |
| ANT-191 | 184 | 0.907 | 5.217 | 0.917 | 4.992 | 5.105 |
| ANT-193 | 186 | 0.582 | 3.345 | 0.701 | 3.815 | 3.580 |
| ANT-194 | 187 | 0.722 | 4.152 | 0.794 | 4.324 | 4.238 |
| ANT-195 | 188 | 0.781 | 4.490 | 0.847 | 4.611 | 4.550 |
| ANT-196 | 189 | 0.859 | 4.940 | 0.968 | 5.268 | 5.104 |
| ANT-200 | 193 | 0.800 | 4.599 | 0.828 | 4.506 | 4.552 |
| ANT-202 | 195 | 0.783 | 4.499 | 0.805 | 4.383 | 4.441 |
| ANT-204 | 197 | 0.980 | 5.636 | 0.861 | 4.689 | 5.163 |
| ANT-205 | 198 | 0.902 | 5.187 | 0.918 | 5.001 | 5.094 |
| ANT-207 | 200 | 0.773 | 4.446 | 0.770 | 4.192 | 4.319 |
| ANT-211 | 204 | 0.970 | 4.446 | 1.097 | 4.271 | 4.359 |
| ANT-212 | 205 | 1.005 | 4.605 | 1.221 | 4.756 | 4.681 |
| ANT-213 | 206 | 0.791 | 3.624 | 0.920 | 3.583 | 3.603 |
| ANT-214 | 207 | 0.831 | 3.808 | 0.966 | 3.761 | 3.784 |
| ANT-215 | 208 | 0.898 | 4.115 | 1.105 | 4.302 | 4.209 |
| ANT-216 | 209 | 0.969 | 4.441 | 1.012 | 3.941 | 4.191 |
| ANT-217 | 210 | 0.933 | 4.273 | 0.996 | 3.880 | 4.076 |
| ANT-218 | 211 | 0.953 | 4.367 | 0.992 | 3.865 | 4.116 |
| ANT-219 | 212 | 0.856 | 3.920 | 0.883 | 3.440 | 3.680 |
| ANT-220 | 213 | 0.863 | 3.953 | 0.954 | 3.717 | 3.835 |
| ANT-221 | 214 | 0.803 | 3.677 | 0.831 | 3.235 | 3.456 |
| ANT-222 | 215 | 1.081 | 4.952 | 1.160 | 4.519 | 4.736 |
| ANT-223 | 216 | 1.077 | 4.933 | 1.111 | 4.327 | 4.630 |
| ANT-224 | 217 | 0.815 | 3.732 | 0.810 | 3.156 | 3.444 |
| ANT-225 | 218 | 1.064 | 4.876 | 1.259 | 4.903 | 4.889 |
| ANT-226 | 219 | 0.996 | 4.564 | 1.094 | 4.262 | 4.413 |
| ANT-227 | 220 | 1.068 | 4.893 | 1.236 | 4.815 | 4.854 |
| ANT-228 | 221 | 1.026 | 4.699 | 1.125 | 4.383 | 4.541 |
| ANT-229 | 222 | 0.836 | 3.831 | 0.913 | 3.556 | 3.694 |
| ANT-230 | 223 | 1.002 | 4.589 | 1.146 | 4.464 | 4.526 |
| ANT-231 | 224 | 1.056 | 4.836 | 1.113 | 4.334 | 4.585 |

### Example 4: Design 2 of Gapmer (Oligonucleotide) to be Tested

A gapmer (oligonucleotide) to be tested was designed as an antisense oligonucleotide (ASO) targeting an mRNA of human TDP-43 (Gen Bank: NM_007375.4, SEQ ID NO: 1) or a pre-mRNA thereof. Sequences of oligonucleotides expressed by DNA bases are shown in Tables 6 to 9. Here, “Start site in SEQ ID NO: 1” indicates the number (position), from the 5′ end of the base sequence of SEQ ID NO: 1, of the base at the 5′ end of the target region of each ASO. Also, “End site in SEQ ID NO: 1” indicates the number (position), from the 5′ end of the base sequence of SEQ ID NO: 1, of the base at the 3′ end of the target region of each ASO.

In Tables 6 to 7, all oligonucleotides are 5-10-5 gapmers, wing regions thereof are all ribonucleotides having 2′-O-MOE modification, gap regions are all deoxyribonucleotides, and bonds between nucleosides are all phosphorothioates (P═S). Besides, regarding the gapmers of Tables 6 to 7, C (cytosine) in the gap region and the wing regions is in reality methyl C, and T in the wing regions is in reality T.

TABLE 6
6 Gapmer (oligonucleotide) to be tested having 2′-O-MOE group in wing nucleoside

| | | | | | SEQ |
|---|---|---|---|---|---|
| ASO base sequence (DNA) | Start site in | End site in | Target | ASO | ID |
| 5′ to 3′ | SEQ ID NO: 1 | SEQ ID NO: 1 | exon | name | NO: |
| GCATCTGTCTCATCCATTTT | 352 | 371 | 3 | ANT-233 | 231 |
| AGCATCTGTCTCATCCATTT | 353 | 372 | 3 | ANT-234 | 232 |
| AAGCATCTGTCTCATCCATT | 354 | 373 | 3 | ANT-235 | 233 |
| GAAGCATCTGTCTCATCCAT | 355 | 374 | 3 | ANT-236 | 234 |
| CTTTTCACTTTCACTGCTGA | 376 | 395 | 3 | ANT-373 | 235 |
| CTGGACTGCTCTTTTCACTT | 386 | 405 | 3 | ANT-374 | 236 |
| GTTTTCTGGACTGCTCTTTT | 391 | 410 | 3 | ANT-268 | 237 |
| GATGTTTTCTGGACTGCTCT | 394 | 413 | 3 | ANT-237 | 238 |
| GAGACCCAACACTATTAAAT | 416 | 435 | 3 | ANT-376 | 239 |
| TTTTCCATGGGAGACCCAAC | 426 | 445 | 3 | ANT-377 | 240 |
| GGTTGTTTTCCATGGGAGAC | 431 | 450 | 3 | ANT-270 | 241 |
| TTAAGATCTTTCTTGACCTG | 502 | 521 | 3 | ANT-243 | 242 |
| AGTCTTAAGATCTTTCTTGA | 506 | 525 | 4 | ANT-378 | 243 |
| TGACCAGTCTTAAGATCTTT | 511 | 530 | 4 | ANT-244 | 244 |
| TTGAATGACCAGTCTTAAGA | 516 | 535 | 4 | ANT-359 | 245 |
| TTTGAATGACCAGTCTTAAG | 517 | 536 | 4 | ANT-476 | 246 |
| CTTTGAATGACCAGTCTTAA | 518 | 537 | 4 | ANT-423 | 247 |
| CCTTTGAATGACCAGTCTTA | 519 | 538 | 4 | ANT-424 | 248 |
| CCCTTTGAATGACCAGTCTT | 520 | 539 | 4 | ANT-425 | 249 |
| ACCCCTTTGAATGACCAGTC | 522 | 541 | 4 | ANT-426 | 250 |
| AACCCCTTTGAATGACCAGT | 523 | 542 | 4 | ANT-427 | 251 |
| AAACCCCTTTGAATGACCAG | 524 | 543 | 4 | ANT-428 | 252 |
| CAAACCCCTTTGAATGACCA | 525 | 544 | 4 | ANT-471 | 253 |
| CCAAACCCCTTTGAATGACC | 526 | 545 | 4 | ANT-360 | 254 |
| GCCAAACCCCTTTGAATGAC | 527 | 546 | 4 | ANT-472 | 255 |
| AGCCAAACCCCTTTGAATGA | 528 | 547 | 4 | ANT-473 | 256 |
| AAGCCAAACCCCTTTGAATG | 529 | 548 | 4 | ANT-474 | 257 |
| CAAAGCCAAACCCCTTTGAA | 531 | 550 | 4 | ANT-271 | 258 |
| TTCATATTCCGTAAAACGAA | 551 | 570 | 4 | ANT-245 | 259 |
| TGTGTTTCATATTCCGTAAA | 556 | 575 | 4 | ANT-379 | 260 |
| TACTTTCACTTGTGTTTCAT | 566 | 585 | 4 | ANT-380 | 261 |
| GACATTACTTTCACTTGTGT | 571 | 590 | 4 | ANT-272 | 262 |
| CGCTGTGACATTACTTTCAC | 577 | 596 | 4 | ANT-246 | 263 |
| ATCATATGTCGCTGTGACAT | 586 | 605 | 4 | ANT-381 | 264 |
| CATCTATCATATGTCGCTGT | 591 | 610 | 4 | ANT-247 | 265 |
| CTTTGCTTAGAATTAGGAAG | 631 | 650 | 4 | ANT-248 | 266 |
| GCTTCTCAAAGGCTCATCTT | 653 | 672 | 5 | ANT-382 | 267 |
| TTTCTGCTTCTCAAAGGCTC | 658 | 677 | 5 | ANT-238 | 268 |
| ACACTTTTCTGCTTCTCAAA | 663 | 682 | 5 | ANT-383 | 269 |
| GTCATGTCCTCTGTACAGCG | 691 | 710 | 5 | ANT-273 | 270 |
| CCTCAGTCATGTCCTCTGTA | 696 | 715 | 5 | ANT-361 | 271 |
| CGCAGCTCATCCTCAGTCAT | 706 | 725 | 5 | ANT-362 | 272 |
| ACTCCCGCAGCTCATCCTCA | 711 | 730 | 5 | ANT-274 | 273 |
| TCCTCTCCACAAAGAGACTG | 823 | 842 | 6 | ANT-239 | 274 |
| TGTGCTTAGGTTCGGCATTG | 876 | 895 | 6 | ANT-363 | 275 |
| TTGTGCTTAGGTTCGGCATT | 877 | 896 | 6 | ANT-477 | 276 |
| ATTGTGCTTAGGTTCGGCAT | 878 | 897 | 6 | ANT-465 | 277 |
| TATTGTGCTTAGGTTCGGCA | 879 | 898 | 6 | ANT-466 | 278 |
| CTATTGTGCTTAGGTTCGGC | 880 | 899 | 6 | ANT-467 | 279 |
| TGCTATTGTGCTTAGGTTCG | 882 | 901 | 6 | ANT-468 | 280 |
| TTGCTATTGTGCTTAGGTTC | 883 | 902 | 6 | ANT-469 | 281 |
| ATTGCTATTGTGCTTAGGTT | 884 | 903 | 6 | ANT-470 | 282 |
| TATTGCTATTGTGCTTAGGT | 885 | 904 | 6 | ANT-478 | 283 |
| CTATTGCTATTGTGCTTAGG | 886 | 905 | 6 | ANT-364 | 284 |
| ACTGTCTATTGCTATTGTGC | 891 | 910 | 6 | ANT-275 | 285 |
| AAATCTTCCACTTCTTTCTA | 911 | 930 | 6 | ANT-276 | 286 |
| TCCCAAAGCCACCTGGATTA | 936 | 955 | 6 | ANT-384 | 287 |
| CAAATCCACCCTGATTCCCA | 951 | 970 | 6 | ANT-278 | 288 |
| ACTGAACAAACCAATTTTGA | 1446 | 1465 | 3′UTR | ANT-394 | 289 |
| TCCACACTGAACAAACCAAT | 1451 | 1470 | 3′UTR | ANT-294 | 290 |
| TACTCCACACTGAACAAACC | 1454 | 1473 | 3′UTR | ANT-307 | 291 |
| TGCTGAATATACTCCACACT | 1463 | 1482 | 3′UTR | ANT-308 | 292 |
| AAAAATACTGCTGAATATAC | 1471 | 1490 | 3′UTR | ANT-295 | 293 |
| TTTTTCTAAAGAAAAATGTC | 1491 | 1510 | 3′UTR | ANT-290 | 294 |
| CTTCCTTTTTCTAAAGAAAA | 1496 | 1515 | 3′UTR | ANT-395 | 295 |
| TCCTTTAGCTCTTCCTTTTT | 1506 | 1525 | 3′UTR | ANT-396 | 296 |
| AAAATTCCTTTAGCTCTTCC | 1511 | 1530 | 3′UTR | ANT-291 | 297 |
| CTTATAAAATTCCTTTAGCT | 1516 | 1535 | 3′UTR | ANT-397 | 298 |
| CTCAATATTTCAACCTTTCA | 1546 | 1565 | 3′UTR | ANT-398 | 299 |
| AACCACTCAATATTTCAACC | 1551 | 1570 | 3′UTR | ANT-282 | 300 |
| CTTTCAACCACTCAATATTT | 1556 | 1575 | 3′UTR | ANT-365 | 301 |
| CAGCAGTTCACTTTCAACCA | 1566 | 1585 | 3′UTR | ANT-366 | 302 |
| GCAAACAGCAGTTCACTTTC | 1571 | 1590 | 3′UTR | ANT-283 | 303 |
| TTGACAAGTCCAGGGATAAA | 1646 | 1665 | 3′UTR | ANT-367 | 304 |
| CTTGACAAGTCCAGGGATAA | 1647 | 1666 | 3′UTR | ANT-479 | 305 |
| ACTTGACAAGTCCAGGGATA | 1648 | 1667 | 3′UTR | ANT-429 | 306 |
| CACTTGACAAGTCCAGGGAT | 1649 | 1668 | 3′UTR | ANT-430 | 307 |
| TCACTTGACAAGTCCAGGGA | 1650 | 1669 | 3′UTR | ANT-431 | 308 |
| TTCACTTGACAAGTCCAGGG | 1651 | 1670 | 3′UTR | ANT-296 | 309 |
| ATTCACTTGACAAGTCCAGG | 1652 | 1671 | 3′UTR | ANT-432 | 310 |
| AATTCACTTGACAAGTCCAG | 1653 | 1672 | 3′UTR | ANT-433 | 311 |
| GAATTCACTTGACAAGTCCA | 1654 | 1673 | 3′UTR | ANT-434 | 312 |
| AAGAATTCACTTGACAAGTC | 1656 | 1675 | 3′UTR | ANT-368 | 313 |
| TGAACATGCAAAGAATTCAC | 1666 | 1685 | 3′UTR | ANT-399 | 314 |
| CGTTTTGAACATGCAAAGAA | 1671 | 1690 | 3′UTR | ANT-297 | 315 |
| GTTTCCGTTTTGAACATGCA | 1676 | 1695 | 3′UTR | ANT-400 | 316 |
| CATGATCTCCTAAAAGGAGA | 1771 | 1790 | 3′UTR | ANT-298 | 317 |
| GACACCATGATCTCCTAAAA | 1776 | 1795 | 3′UTR | ANT-401 | 318 |
| CAAACACTGTGACACCATGA | 1786 | 1805 | 3′UTR | ANT-402 | 319 |
| AGAACCAAACACTGTGACAC | 1791 | 1810 | 3′UTR | ANT-299 | 320 |
| GAATTCTTGAATGAGAAAGC | 1971 | 1990 | 3′UTR | ANT-300 | 321 |
| ATGACGAATTCTTGAATGAG | 1976 | 1995 | 3′UTR | ANT-403 | 322 |
| GTGATGCGTGATGACGAATT | 1986 | 2005 | 3′UTR | ANT-404 | 323 |
| GGCCTGTGATGCGTGATGAC | 1991 | 2010 | 3′UTR | ANT-301 | 324 |
| TGATACGACTCCATGAAATA | 2053 | 2072 | 3′UTR | ANT-309 | 325 |
| TTGATACGACTCCATGAAAT | 2054 | 2073 | 3′UTR | ANT-310 | 326 |
| GTTGATACGACTCCATGAAA | 2055 | 2074 | 3′UTR | ANT-311 | 327 |
| CGTTGATACGACTCCATGAA | 2056 | 2075 | 3′UTR | ANT-312 | 328 |
| TAGCGTTGATACGACTCCAT | 2059 | 2078 | 3′UTR | ANT-313 | 329 |
| ATAGCGTTGATACGACTCCA | 2060 | 2079 | 3′UTR | ANT-314 | 330 |
| TCATAGCGTTGATACGACTC | 2062 | 2081 | 3′UTR | ANT-315 | 331 |
| TTCATAGCGTTGATACGACT | 2063 | 2082 | 3′UTR | ANT-316 | 332 |
| CGTTCATAGCGTTGATACGA | 2065 | 2084 | 3′UTR | ANT-249 | 333 |
| CCTTGCGTTCATAGCGTTGA | 2070 | 2089 | 3′UTR | ANT-435 | 334 |
| GCCTTGCGTTCATAGCGTTG | 2071 | 2090 | 3′UTR | ANT-284 | 335 |
| AGCCTTGCGTTCATAGCGTT | 2072 | 2091 | 3′UTR | ANT-436 | 336 |
| ACAGCCTTGCGTTCATAGCG | 2074 | 2093 | 3′UTR | ANT-437 | 337 |
| CACAGCCTTGCGTTCATAGC | 2075 | 2094 | 3′UTR | ANT-438 | 338 |
| TCACAGCCTTGCGTTCATAG | 2076 | 2095 | 3′UTR | ANT-439 | 339 |
| ATCACAGCCTTGCGTTCATA | 2077 | 2096 | 3′UTR | ANT-317 | 340 |
| TATCACAGCCTTGCGTTCAT | 2078 | 2097 | 3′UTR | ANT-318 | 341 |
| CATATCACAGCCTTGCGTTC | 2080 | 2099 | 3′UTR | ANT-319 | 342 |
| TCCATATCACAGCCTTGCGT | 2082 | 2101 | 3′UTR | ANT-480 | 343 |
| GTTCCATATCACAGCCTTGC | 2084 | 2103 | 3′UTR | ANT-481 | 344 |
| GGTTCCATATCACAGCCTTG | 2085 | 2104 | 3′UTR | ANT-320 | 345 |
| TGGTTCCATATCACAGCCTT | 2086 | 2105 | 3′UTR | ANT-321 | 346 |
| CCTTCTGGTTCCATATCACA | 2091 | 2110 | 3′UTR | ANT-250 | 347 |
| GCCTTCTGGTTCCATATCAC | 2092 | 2111 | 3′UTR | ANT-322 | 348 |
| GACAGCCTTCTGGTTCCATA | 2096 | 2115 | 3′UTR | ANT-405 | 349 |
| CAAAAGTTCAGACAGCCTTC | 2106 | 2125 | 3′UTR | ANT-406 | 350 |
| GGTTTCAAAAGTTCAGACAG | 2111 | 2130 | 3′UTR | ANT-302 | 351 |
| CCACCATCAATCCCACACAA | 2131 | 2150 | 3′UTR | ANT-251 | 352 |
| CACCACCATCAATCCCACAC | 2133 | 2152 | 3′UTR | ANT-323 | 353 |
| GCACCACCATCAATCCCACA | 2134 | 2153 | 3′UTR | ANT-440 | 354 |
| GGCACCACCATCAATCCCAC | 2135 | 2154 | 3′UTR | ANT-441 | 355 |
| CGGCACCACCATCAATCCCA | 2136 | 2155 | 3′UTR | ANT-324 | 356 |
| TCGGCACCACCATCAATCCC | 2137 | 2156 | 3′UTR | ANT-325 | 357 |
| CTCGGCACCACCATCAATCC | 2138 | 2157 | 3′UTR | ANT-442 | 358 |
| CCTCGGCACCACCATCAATC | 2139 | 2158 | 3′UTR | ANT-443 | 359 |
| GCCTCGGCACCACCATCAAT | 2140 | 2159 | 3′UTR | ANT-444 | 360 |
| ATGCCTCGGCACCACCATCA | 2142 | 2161 | 3′UTR | ANT-326 | 361 |
| CATGCCTCGGCACCACCATC | 2143 | 2162 | 3′UTR | ANT-327 | 362 |
| TCATGCCTCGGCACCACCAT | 2144 | 2163 | 3′UTR | ANT-328 | 363 |
| TTCATGCCTCGGCACCACCA | 2145 | 2164 | 3′UTR | ANT-329 | 364 |
| TTTCATGCCTCGGCACCACC | 2146 | 2165 | 3′UTR | ANT-445 | 365 |
| CTTTCATGCCTCGGCACCAC | 2147 | 2166 | 3′UTR | ANT-252 | 366 |
| CCTTTCATGCCTCGGCACCA | 2148 | 2167 | 3′UTR | ANT-446 | 367 |
| GCCTTTCATGCCTCGGCACC | 2149 | 2168 | 3′UTR | ANT-447 | 368 |
| AGCCTTTCATGCCTCGGCAC | 2150 | 2169 | 3′UTR | ANT-448 | 369 |
| TAGCCTTTCATGCCTCGGCA | 2151 | 2170 | 3′UTR | ANT-285 | 370 |
| CTAGCCTTTCATGCCTCGGC | 2152 | 2171 | 3′UTR | ANT-330 | 371 |
| TACTAGCCTTTCATGCCTCG | 2154 | 2173 | 3′UTR | ANT-331 | 372 |
| ATACTAGCCTTTCATGCCTC | 2155 | 2174 | 3′UTR | ANT-332 | 373 |
| CTCATACTAGCCTTTCATGC | 2158 | 2177 | 3′UTR | ANT-333 | 374 |
| GCTCATACTAGCCTTTCATG | 2159 | 2178 | 3′UTR | ANT-482 | 375 |
| CGCTCATACTAGCCTTTCAT | 2160 | 2179 | 3′UTR | ANT-334 | 376 |
| CTCGCTCATACTAGCCTTTC | 2162 | 2181 | 3′UTR | ANT-483 | 377 |
| CTTTTCTCGCTCATACTAGC | 2167 | 2186 | 3′UTR | ANT-335 | 378 |
| CCTTTTCTCGCTCATACTAG | 2168 | 2187 | 3′UTR | ANT-336 | 379 |
| TCTCCTTTTCTCGCTCATAC | 2171 | 2190 | 3′UTR | ANT-253 | 380 |
| CGCTCTCTCCTTTTCTCGCT | 2176 | 2195 | 3′UTR | ANT-407 | 381 |
| GCGCTCTCTCCTTTTCTCGC | 2177 | 2196 | 3′UTR | ANT-484 | 382 |
| ACGCGCTCTCTCCTTTTCTC | 2179 | 2198 | 3′UTR | ANT-485 | 383 |
| TCTCTGCACGCGCTCTCTCC | 2186 | 2205 | 3′UTR | ANT-408 | 384 |
| CAAGTCTCTGCACGCGCTCT | 2190 | 2209 | 3′UTR | ANT-338 | 385 |
| ACCAAGTCTCTGCACGCGCT | 2192 | 2211 | 3′UTR | ANT-339 | 386 |
| CCACCAAGTCTCTGCACGC | 2194 | 2213 | 3′UTR | ANT-340 | 387 |
| G | | | | | |
| AAAATATCCATTATGCACCA | 2211 | 2230 | 3′UTR | ANT-255 | 388 |
| CAAGTTAAAAAATATCCATT | 2219 | 2238 | 3′UTR | ANT-256 | 389 |
| GACACATCTCGCCAAGTTAA | 2231 | 2250 | 3′UTR | ANT-257 | 390 |
| ACCAAAGCCACAGGATTGAG | 2252 | 2271 | 3′UTR | ANT-258 | 391 |
| CTGCACACTCTCTCACCAAA | 2266 | 2285 | 3′UTR | ANT-369 | 392 |
| CTCTGCACACTCTCTCACCA | 2268 | 2287 | 3′UTR | ANT-449 | 393 |
| TCTCTGCACACTCTCTCACC | 2269 | 2288 | 3′UTR | ANT-450 | 394 |
| CTCTCTGCACACTCTCTCAC | 2270 | 2289 | 3′UTR | ANT-451 | 395 |
| GCTCTCTGCACACTCTCTCA | 2271 | 2290 | 3′UTR | ANT-286 | 396 |
| TGCTCTCTGCACACTCTCTC | 2272 | 2291 | 3′UTR | ANT-452 | 397 |
| TTGCTCTCTGCACACTCTCT | 2273 | 2292 | 3′UTR | ANT-453 | 398 |
| ATTGCTCTCTGCACACTCTC | 2274 | 2293 | 3′UTR | ANT-454 | 399 |
| TATCATTGCTCTCTGCACAC | 2278 | 2297 | 3′UTR | ANT-455 | 400 |
| CTATCATTGCTCTCTGCACA | 2279 | 2298 | 3′UTR | ANT-341 | 401 |
| GCTATCATTGCTCTCTGCAC | 2280 | 2299 | 3′UTR | ANT-342 | 402 |
| TTGCTATCATTGCTCTCTGC | 2282 | 2301 | 3′UTR | ANT-456 | 403 |
| TTTGCTATCATTGCTCTCTG | 2283 | 2302 | 3′UTR | ANT-457 | 404 |
| ATTTGCTATCATTGCTCTCT | 2284 | 2303 | 3′UTR | ANT-458 | 405 |
| TTATTTGCTATCATTGCTCT | 2286 | 2305 | 3′UTR | ANT-370 | 406 |
| GTACATTATTTGCTATCATT | 2291 | 2310 | 3′UTR | ANT-287 | 407 |
| CATTCGTACATTATTTGCTA | 2296 | 2315 | 3′UTR | ANT-409 | 408 |
| CATATCAGTAACAGAAGAGA | 2471 | 2490 | 3′UTR | ANT-288 | 409 |
| ACTTACATATCAGTAACAGA | 2476 | 2495 | 3′UTR | ANT-371 | 410 |
| CACTTACATATCAGTAACAG | 2477 | 2496 | 3′UTR | ANT-486 | 411 |
| ACACTTACATATCAGTAACA | 2478 | 2497 | 3′UTR | ANT-459 | 412 |
| CACACTTACATATCAGTAAC | 2479 | 2498 | 3′UTR | ANT-460 | 413 |
| CCACACTTACATATCAGTAA | 2480 | 2499 | 3′UTR | ANT-461 | 414 |
| TGCCACACTTACATATCAGT | 2482 | 2501 | 3′UTR | ANT-462 | 415 |
| TTGCCACACTTACATATCAG | 2483 | 2502 | 3′UTR | ANT-463 | 416 |
| ATTGCCACACTTACATATCA | 2484 | 2503 | 3′UTR | ANT-464 | 417 |
| ACATTGCCACACTTACATAT | 2486 | 2505 | 3′UTR | ANT-372 | 418 |
| AGTTCACATTGCCACACTTA | 2491 | 2510 | 3′UTR | ANT-289 | 419 |

TABLE 7
Gapmer (oligonucleotide) to be tested having 2′-O-MOE group in wing nucleoside

| | | | | | SEQ |
|---|---|---|---|---|---|
| ASO base sequence (DNA) | Start site in | End site in | Target | ASO | ID |
| 5′ to 3′ | SEQ ID NO: 1 | SEQ ID NO: 1 | exon | name | NO: |
| GCCCCTGGAAACTGGGCTG | 196 | 215 | 2 | ANT-232 | 475 |
| T | | | | | |
| CACTTTCACTGCTGATGAAG | 371 | 390 | 3 | ANT-267 | 476 |
| ACTATTAAATCGGATGTTTT | 406 | 425 | 3 | ANT-375 | 477 |
| CCAACACTATTAAATCGGAT | 411 | 430 | 3 | ANT-269 | 478 |
| ACCTGGATTACCACCAAATC | 926 | 945 | 6 | ANT-303 | 479 |
| CACCTGGATTACCACCAAAT | 927 | 946 | 6 | ANT-304 | 480 |
| CCACCTGGATTACCACCAAA | 928 | 947 | 6 | ANT-305 | 481 |
| GCCACCTGGATTACCACCAA | 929 | 948 | 6 | ANT-306 | 482 |
| AAGCCACCTGGATTACCACC | 931 | 950 | 6 | ANT-277 | 483 |
| CATACCCCAACTGCTCTGTA | 1091 | 1110 | 6 | ANT-279 | 484 |
| TGGCTGGCTAACATGCCCAT | 1111 | 1130 | 6 | ANT-240 | 485 |
| CCTGACTGGTTCTGCTGGCT | 1126 | 1145 | 6 | ANT-385 | 486 |
| ATGGGCCTGACTGGTTCTG | 1131 | 1150 | 6 | ANT-280 | 487 |
| C | | | | | |
| ACCCGATGGGCCTGACTGG | 1136 | 1155 | 6 | ANT-386 | 488 |
| T | | | | | |
| TTTGGTTATTACCCGATGGG | 1146 | 1165 | 6 | ANT-387 | 489 |
| TTGGTTTTGGTTATTACCCG | 1151 | 1170 | 6 | ANT-281 | 490 |
| TTGCCTTGGTTTTGGTTATT | 1156 | 1175 | 6 | ANT-388 | 491 |
| CCATTAAAACCACTGCCCGA | 1279 | 1298 | 6 | ANT-241 | 492 |
| TGCTTGAGCCAAAGCCTCCA | 1296 | 1315 | 6 | ANT-389 | 493 |
| TTAGAATCCATGCTTGAGCC | 1306 | 1325 | 6 | ANT-390 | 494 |
| GAAGACTTAGAATCCATGCT | 1312 | 1331 | 6 | ANT-242 | 495 |
| AGCCAGAAGACTTAGAATCC | 1317 | 1336 | 6 | ANT-391 | 496 |
| CCAACCAACCACAACCCCA | 1351 | 1370 | 3′UTR | ANT-292 | 497 |
| C | | | | | |
| CTATACCAACCAACCACAAC | 1356 | 1375 | 3′UTR | ANT-392 | 498 |
| TCCCACCATTCTATACCAAC | 1366 | 1385 | 3′UTR | ANT-393 | 499 |
| TGAATTCCCACCATTCTATA | 1371 | 1390 | 3′UTR | ANT-293 | 500 |
| AAGTCTCTGCACGCGCTCTC | 2189 | 2208 | 3′UTR | ANT-337 | 501 |
| CCAAGTCTCTGCACGCGCT | 2191 | 2210 | 3′UTR | ANT-254 | 502 |
| C | | | | | |

In Table 8, all oligonucleotides are 5-10-5 gapmers, wing regions thereof are all ribonucleotides having 2′-O-MOE modification, gap regions are deoxyribonucleotides or ribonucleotides having 2′-OMe modification or 2′-O-MOE modification. Besides, bonds between nucleosides are phosphorothioates (P═S) or phosphodiester (P═O). In Table 8, a ribonucleotide having 2′-O-MOE modification is underlined, and a ribonucleotide having a 2′-OMe modification is double underlined. A bond between nucleosides is a phosphorothioate bond unless otherwise stated, and when a bond between nucleosides is a phosphodiester bond, the bond is shown as “\hat{ }” inserted between bases. In the gapmers of Table 8, C (cytosine) in the gap region and the wing regions is actually methyl C. C in a ribonucleotide having a 2′-OMe group is not methyl C but C. T in the wing regions is actually T, and T in a 2′-OMe group of the gap region is actually U.

TABLE 8
Gapmer (oligonucleotide) to be tested comprising phosphodiester bond in bonds between
wing nucleosides

| | Start site | End site in | | | SEQ |
|---|---|---|---|---|---|
| | in SEQ ID | SEQ ID | Target | ASO | ID |
| ASO base sequence (DNA) 5′ to 3′ | NO: 1 | NO: 1 | exon | name | NO: |
| CC^{∧}CC^{∧}TTTGAATGACCA^{∧}GT^{∧}CT | 521 | 540 | 4 | ANT-491 | 25 |
| CC^{∧}CC^{∧}TTT^{∧}GAATGACCA^{∧}GTACT | 521 | 540 | 4 | ANT-492 | 422 |
| CC^{∧}CC^{∧}TT^{∧}TGAATGACCA^{∧}GT^{∧}CT | 521 | 540 | 4 | ANT-493 | 422 |
| GC^{∧}TA^{∧}TTGTGCTTAGGT^{∧}TC^{∧}GG | 881 | 900 | 6 | ANT-494 | 42 |
| GC^{∧}TA^{∧}TTG^{∧}TGCTTAGGT^{∧}TC^{∧}GG | 881 | 900 | 6 | ANT-495 | 423 |
| GC^{∧}TA^{∧}TT^{∧}GTGCTTAGGT^{∧}TC^{∧}GG | 881 | 900 | 6 | ANT-496 | 423 |
| GA^{∧}TT^{∧}ACCACCAAATCT^{∧}TC^{∧}CA | 921 | 940 | 6 | ANT-349 | 44 |
| CT^{∧}GA^{∧}ATATACTCCACA^{∧}CT^{∧}GA | 1461 | 1480 | 3′UTR | ANT-353 | 74 |
| TT^{∧}CA^{∧}CTTGACAAGTCC^{∧}AG^{∧}GG | 1651 | 1670 | 3′UTR | ANT-497 | 309 |
| TT^{∧}CA^{∧}CTT^{∧}GACAAGTCC^{∧}AG^{∧}GG | 1651 | 1670 | 3′UTR | ANT-498 | 424 |
| TT^{∧}CA^{∧}CT^{∧}TGACAAGTCC^{∧}AG^{∧}GG | 1651 | 1670 | 3′UTR | ANT-499 | 424 |
| CA^{∧}TA^{∧}GCGTTGATACGA^{∧}CT^{∧}CC | 2061 | 2080 | 3′UTR | ANT-354 | 105 |
| CA^{∧}G^{∧}C^{∧}CTTGCGTTCATA^{∧}G^{∧}C^{∧}GT | 2073 | 2092 | 3′UTR | ANT-263 | 106 |
| CA^{∧}GC^{∧}CTTGCGTTCATA^{∧}GC^{∧}GT | 2073 | 2092 | 3′UTR | ANT-264 | 106 |
| CC^{∧}AT^{∧}ATCACAGCCTTG^{∧}CG^{∧}TT | 2081 | 2100 | 3′UTR | ANT-350 | 107 |
| CG^{∧}GC^{∧}ACCACCATCAAT^{∧}CC^{∧}CA | 2136 | 2155 | 3′UTR | ANT-411 | 356 |
| TG^{∧}CC^{∧}TCGGCACCACCA^{∧}TC^{∧}AA | 2141 | 2160 | 3′UTR | ANT-351 | 110 |
| TT^{∧}CA^{∧}TGCCTCGGCACC^{∧}AC^{∧}CA | 2145 | 2164 | 3′UTR | ANT-475 | 364 |
| TT^{∧}CA^{∧}TGC^{∧}CTCGGCACC^{∧}AC^{∧}CA | 2145 | 2164 | 3′UTR | ANT-487 | 425 |
| TT^{∧}CA^{∧}TG^{∧}CCTCGGCACC^{∧}AC^{∧}CA | 2145 | 2164 | 3′UTR | ANT-488 | 425 |
| TC^{∧}GC^{∧}TCATACTAGCCT^{∧}TT^{∧}CA | 2161 | 2180 | 3′UTR | ANT-352 | 112 |
| TC^{∧}GC^{∧}TCA^{∧}TACTAGCCT^{∧}TT^{∧}CA | 2161 | 2180 | 3′UTR | ANT-489 | 426 |
| TC^{∧}GC^{∧}TC^{∧}ATACTAGCCT^{∧}TT^{∧}CA | 2161 | 2180 | 3′UTR | ANT-490 | 426 |
| AC^{∧}C^{∧}A^{∧}CCAAGTCTCTGC^{∧}A^{∧}C^{∧}GC | 2195 | 2214 | 3′UTR | ANT-265 | 114 |
| AC^{∧}CA^{∧}CCAAGTCTCTGC^{∧}AC^{∧}GC | 2195 | 2214 | 3′UTR | ANT-266 | 114 |
| AC^{∧}CA^{∧}CCAAGTCTCTGC^{∧}AC^{∧}GC | 2195 | 2214 | 3′UTR | ANT-343 | 427 |
| AC^{∧}CA^{∧}CCA^{∧}AGTCTCTGC^{∧}AC^{∧}GC | 2195 | 2214 | 3′UTR | ANT-344 | 427 |
| AC^{∧}CA^{∧}CC^{∧}AAGTCTCTGC^{∧}AC^{∧}GC | 2195 | 2214 | 3′UTR | ANT-345 | 427 |
| AC^{∧}CA^{∧}CCAAGTCTCTGC^{∧}AC^{∧}GC | 2195 | 2214 | 3′UTR | ANT-346 | 427 |
| AC^{∧}CA^{∧}CCA^{∧}AGTCTCTGC^{∧}AC^{∧}GC | 2195 | 2214 | 3′UTR | ANT-347 | 427 |
| AC^{∧}CA^{∧}CC^{∧}AAGTCTCTGC^{∧}AC^{∧}GC | 2195 | 2214 | 3′UTR | ANT-348 | 427 |
| GC^{∧}TC^{∧}TCTGCACACTCT^{∧}CT^{∧}CA | 2271 | 2290 | 3′UTR | ANT-410 | 396 |
| GC^{∧}TA^{∧}TCATTGCTCTCT^{∧}GC^{∧}AC | 2280 | 2299 | 3′UTR | ANT-500 | 402 |
| GC^{∧}TA^{∧}TCA^{∧}TTGCTCTCT^{∧}GC^{∧}AC | 2280 | 2299 | 3′UTR | ANT-501 | 428 |
| GC^{∧}TA^{∧}TC^{∧}ATTGCTCTCT^{∧}GC^{∧}AC | 2280 | 2299 | 3′UTR | ANT-502 | 428 |

In Table 9, all oligonucleotides are 5-10-5 gapmers, wing regions thereof are all ribonucleotides having 2′-O-MOE modification, gap regions are deoxyribonucleotides or ribonucleotides having 2′-OMe modification or 2′-O-MOE modification. Besides, bonds between nucleosides are phosphorothioates (P═S) or phosphodiesters (P═O). In Table 9, a ribonucleotide having 2′-O-MOE modification is underlined, and a ribonucleotide having a 2′-OMe modification is double underlined. A bond between nucleosides is a phosphorothioate bond unless otherwise stated, and when a bond between nucleosides is a phosphodiester bond, the bond is shown as “\hat{ }” inserted between bases. Regarding the gapmers of Table 9, C (cytosine) in the gap region and the wing regions is in reality methyl C. C in a ribonucleotide having a 2′-OMe group is not methyl C but C. T in the wing regions is in reality T, and T in a 2′-OMe group of the gap region is in reality U.

TABLE 9
Gapmer (oligonucleotide) to be tested comprising phosphodiester bond in bonds between
wing nucleosides

| | Start site | End site | | |
|---|---|---|---|---|
| | in SEQ | in SEQ | | |
| ASO base sequence (DNA) 5′ to 3′ | ID NO: 1 | ID NO: 1 | ASO name | SEQ ID NO: |
| AC^{∧}CC^{∧}CTTTGAATGACC^{∧}AG^{∧}TC | 522 | 541 | ANT-503 | 250 |
| AC^{∧}CC^{∧}CTT^{∧}TGAATGACC^{∧}AG^{∧}TC | 522 | 541 | ANT-504 | 429 |
| AC^{∧}CC^{∧}CT^{∧}TTGAATGACC^{∧}AG^{∧}TC | 522 | 541 | ANT-505 | 429 |
| AA^{∧}CC^{∧}CCTTTGAATGAC^{∧}CA^{∧}GT | 523 | 542 | ANT-518 | 251 |
| AA^{∧}CC^{∧}CCT^{∧}TTGAATGAC^{∧}CA^{∧}GT | 523 | 542 | ANT-519 | 430 |
| AA^{∧}CC^{∧}CC^{∧}TTTGAATGAC^{∧}CA^{∧}GT | 523 | 542 | ANT-520 | 430 |
| CC^{∧}AA^{∧}ACCCCTTTGAAT^{∧}GA^{∧}CC | 526 | 545 | ANT-512 | 254 |
| CC^{∧}AA^{∧}ACC^{∧}CCTTTGAAT^{∧}GA^{∧}CC | 526 | 545 | ANT-521 | 431 |
| CC^{∧}AA^{∧}AC^{∧}CCCTTTGAAT^{∧}GA^{∧}CC | 526 | 545 | ANT-522 | 431 |
| GT^{∧}TC^{∧}ACTTTCAACCAC^{∧}TC^{∧}AA | 1561 | 1580 | ANT-524 | 80 |
| GT^{∧}TC^{∧}ACTATTCAACCAC^{∧}TC^{∧}AA | 1561 | 1580 | ANT-525 | 432 |
| GT^{∧}TC^{∧}ACATTTCAACCAC^{∧}TC^{∧}AA | 1561 | 1580 | ANT-526 | 432 |
| CA^{∧}CT^{∧}TGACAAGTCCAG^{∧}GG^{∧}AT | 1649 | 1668 | ANT-514 | 307 |
| CA^{∧}CT^{∧}TGA^{∧}CAAGTCCAG^{∧}GG^{∧}AT | 1649 | 1668 | ANT-527 | 433 |
| CA^{∧}CT^{∧}TG^{∧}ACAAGTCCAG^{∧}GG^{∧}AT | 1649 | 1668 | ANT-528 | 433 |
| TC^{∧}AC^{∧}TTGACAAGTCCA^{∧}GG^{∧}GA | 1650 | 1669 | ANT-506 | 308 |
| TC^{∧}AC^{∧}TTG^{∧}ACAAGTCCA^{∧}GG^{∧}GA | 1650 | 1669 | ANT-507 | 434 |
| TC^{∧}AC^{∧}TT^{∧}GACAAGTCCA^{∧}GG^{∧}GA | 1650 | 1669 | ANT-508 | 434 |
| GC^{∧}AC^{∧}CACCATCAATCC^{∧}CA^{∧}CA | 2134 | 2153 | ANT-523 | 354 |
| GG^{∧}CA^{∧}CCACCATCAATC^{∧}CC^{∧}AC | 2135 | 2154 | ANT-529 | 355 |
| CG^{∧}GC^{∧}ACC^{∧}ACCATCAAT^{∧}CC^{∧}CA | 2136 | 2155 | ANT-516 | 435 |
| CG^{∧}GC^{∧}AC^{∧}CACCATCAAT^{∧}CC^{∧}CA | 2136 | 2155 | ANT-517 | 435 |
| TC^{∧}GC^{∧}TCATACTAGCCT^{∧}TT^{∧}CA | 2161 | 2180 | ANT-515 | 426 |
| TCGCTCATACTAGCCTTTCA | 2161 | 2180 | ANT-509 | 426 |
| TCGCTCA^{∧}TACTAGCCTTTCA | 2161 | 2180 | ANT-510 | 426 |
| TCGCTC^{∧}ATACTAGCCTTTCA | 2161 | 2180 | ANT-511 | 426 |
| GC^{∧}CA^{∧}CACTTACATATC^{∧}AG^{∧}TA | 2481 | 2500 | ANT-513 | 131 |

### Example 5: TDP-43 Gene Knockdown Activity Test of Gapmer (Antisense Oligonucleotide)

### Test Example 2: Measurement of Knockdown Activity (Inhibitory Activity) of Human TDP-43 Gene

Into 3.0×10⁵ A204 cells (human rhabdomyosarcoma cell line, obtained from ATCC), each of gapmers shown in Tables 7 to 9 was introduced in a concentration of 1.5 μM with 4D-Nucleofector™ using SF Cell Line 4D-Nucleofector™X Kit S in accordance with the protocol attached to the kit. DS-130 was used as the program.

After the introduction, the cells were cultured overnight in 0.5 mL of McCoy's 5A Medium (SIGMA) containing a 10% fetal bovine serum (FBS) (Nichirei Corporation) and 1 mM L-glutamine (Gibco) under conditions of 37° C. and 5% CO₂. As substances to be tested, the gapmers described in Example 4 were used.

After washing the resultant cells with PBS (Nacalai Tesque, Inc.) once, buffer RA1 (Takara Bio Inc.) containing 1% 2-mercaptoethanol (Nacalai Tesque, Inc.) was added thereto to dissolve the cells, which were collected on NucleoSpin(registered trademark) Filter (Takara Bio Inc.). The resultant filter was centrifuged at 11,000×g for 1 minute to collect a filtered liquid as a homogenate. Total RNA was extracted from the homogenate with NucleoSpin(registered trademark) RNA (Takara Bio Inc.) in accordance with the protocol attached thereto. The concentration of the extracted total RNA was measured with NanoDrop One (Thermo Fisher).

250 ng of the extracted total RNA was subjected to a reverse transcription (RT) reaction with High Capacity cDNA Reverse Transcription Kit (Applied Biosystems) and a random primer attached to the kit. Specifically, in accordance with the protocol attached to the kit, a reaction solution was prepared. TaKaRa PCR Thermal Cycler Dice Touch (Takara Bio Inc.) was used as the thermal cycler. The program of the RT reaction used was as follows.
25° C., 10 minutes: primer annealing
37° C., 120 minutes: reverse transcription reaction
85° C., 5 minutes: reverse transcriptase inactivation

Thereafter, qPCR was performed with Fast SYBR Green Master Mix (Thermo Fisher Scientific) using the RT reaction product as a template, and the TDP-43 and β-actin RNA expression levels endogenously expressed in the A204 cells were measured. Specifically, a reaction solution was prepared in accordance with the protocol attached to the kit. In the qPCR, Quant Studio 6 Flex Real-Time PCR System (Applied Biosystems) was used. The program of the qPCR used was as follows.
50° C., 2 minutes; and 95° C., 10 minutes: polymerase activation, cDNA thermal denaturation
[95° C., 15 seconds; and 60° C., 1 minute]×40 cycles: PCR Amplification
95° C., 15 seconds; and 60° C., 1 minute to 95° C., 15 seconds (Melting Curve 0.05° C./s): melting curve analysis

Primers used in the detection of the TDP-43 RNA are shown in Table 10. Sequences of 2 portions (exon 2 to exon 3, and 3′ UTR) in TDP-43 were respectively amplified and detected. A primer used in detection of the β-actin RNA of house keeping gene is shown in Table 11.

The TDP-43 RNA detection value was corrected with β-actin RNA detection value of house keeping gene. Besides, a ratio of the TDP-43 RNA corrected value of cells having been subjected to the introduction operation with each gapmer added thereto to the TDP-43 RNA corrected value of cells having been subjected to the introduction operation only with 4D-Nucleofector™ without adding the gapmer thereto (“vehicle”) was calculated, and the thus obtained ratio was used as the TDP-43 RNA expression level to analyze the inhibitory activity of each gapmer. As the RNA expression level, the result obtained by using a primer set for detecting exon 2-3 (RNA expression level (exon 2-3)), and the result obtained by using a primer set for detecting 3′ UTR (RNA expression level (3′ UTR)) are both shown. Besides, as a gapmer for comparison, ANT-10 (SEQ ID NO: 114) that had been confirmed to have activity through a preliminary test (no data shown) was used. Specifically, based on a ratio of the TDP-43 RNA expression level of cells having been subjected to the introduction operation with each gapmer added to the TDP-43 RNA expression level of cells having been subjected to the introduction operation with the gapmer indicated as ANT-10 (SEQ ID NO: 114) added thereto, the RNA expression level ratio to ANT-10 (SEQ ID NO: 114) was calculated. In the calculation, as the TDP-43 RNA expression level of the cells having been subjected to the introduction operation with the gapmer indicated as ANT-10 (SEQ ID NO: 114) added thereto, a value obtained in each assay was used. The results are shown in Tables 12 to 14 ((wherein “Ratio of RNA expression level/ANT-10 (exon 2-3)” indicates the RNA expression level ratio obtained using the primer set for detecting exon 2-3, and “Ratio of RNA expression level/ANT-10 (3′ UTR)” indicates the RNA expression level ratio obtained using the primer set for detecting 3′ UTR. Besides, “Average of ratio of RNA expression level” indicates the average of the ratio of RNA expression level/ANT-10 (exon 2-3) and the ratio of RNA expression level/ANT-10 (3′ UTR). As for ANT-10 (SEQ ID NO: 114), representative values obtained through a plurality of times of assay are shown).

Besides, a similar operation was performed by using ANT-266 (SEQ ID NO: 114) as a gapmer for comparison. Specifically, based on a ratio of the TDP-43 RNA expression level of cells having been subjected to the introduction operation with each gapmer added thereto to the TDP-43 RNA expression level of cells having been subjected to the introduction operation with the gapmer indicated as ANT-266 (SEQ ID NO: 114) added thereto, the RNA expression level ratio to ANT-266 (SEQ ID NO: 114) was calculated. The results are shown in Table 15 ((wherein “Ratio of RNA expression level/ANT-266 (exon 2-3)” indicates the RNA expression level ratio obtained using the primer set for detecting exon 2-3, and “Ratio of RNA expression level/ANT-266 (exon 5-6)” indicates the RNA expression level ratio obtained using the primer set for detecting exon 5-6 (Table 3). “Average of ratio of RNA expression level” indicates the average of the ratio of RNA expression level/ANT-266 (exon 2-3) and the ratio of RNA expression level/ANT-266 (exon 5-6)).

Besides, a similar operation was performed by using ANT-266 (SEQ ID NO: 114) as a gapmer for comparison with the introduction concentration changed to 0.6 M. Specifically, based on a ratio of the TDP-43 RNA expression level of cells having been subjected to the introduction operation with each gapmer added to the TDP-43 RNA expression level of cells having been subjected to the introduction operation with the gapmer indicated as ANT-266 (SEQ ID NO: 114) added thereto, the RNA expression level ratio to ANT-266 (SEQ ID NO: 114) was calculated. The results are shown in Tables 16 to 18 ((wherein “Ratio of RNA expression level/ANT-266 (exon 2-3)” indicates the RNA expression level ratio obtained using the primer set for detecting exon 2-3, and “Ratio of RNA expression level/ANT-266 (3′ UTR)” indicates the RNA expression level ratio obtained using the primer set for detecting 3′ UTR. “Average of ratio of RNA expression level” indicates the average of the ratio of RNA expression level/ANT-266 (exon 2-3) and the ratio of RNA expression level/ANT-266 (3′ UTR)).

TABLE 10
Primer used to detect RNA of Endogenous TDP-43 and recognized exon site

| | Primer set for detection | | | |
|---|---|---|---|---|
| | | SEQ | | SEQ |
| Detected | | ID | | ID |
| exon | Sense primer 5′ to 3′ | NO: | Reverse primer 5′ to 3′ | NO: |
| exon 2-3 | GGGAAATCTGGTGTATGTTGTC | 225 | TTTCAGGTCCTGTTCGGTTG | 226 |
| 3′UTR | GAGAACATGGACTGAGCTTGT | 420 | CAGACATTAGAACTTCCACCCT | 421 |

TABLE 11
Primer used to detect RNA of β-Actin used as gene for correction

| | Primer set for detection | | | |
|---|---|---|---|---|
| | | SEQ | | SEQ |
| Gene for | | ID | | ID |
| correction | Sense primer 5′ to 3′ | NO: | Reverse primer 5′ to 3′ | NO: |
| β-actin | GAAGATCAAGATCATTGCTCCT | 229 | TACTCCTGCTTGCTGATCCA | 230 |

TABLE 12-1
Human TDP-43 gene RNA expression level, etc. in A204 cell treated with respective
gapmers (1.5 μM)

| | | | Ratio of | | Ratio of | Average |
|---|---|---|---|---|---|---|
| | | RNA | RNA | RNA | RNA | of Ratio of |
| | | expression | expression | expression | expression | RNA |
| | | level | level/ANT- | level | level/ANT- | expression |
| ASO name | SEQ ID NO: | (exon2-3) | 10(exon2-3) | (3′UTR) | 10(3′UTR) | level |
| vehicle | — | 1 | 2.556 | 1 | 2.562 | 2.559 |
| ANT-10 | 114 | 0.391 | 1.000 | 0.390 | 1.000 | 1.000 |
| ANT-233 | 231 | 0.856 | 1.838 | 1.049 | 2.341 | 2.089 |
| ANT-234 | 232 | 0.723 | 1.553 | 0.930 | 2.077 | 1.815 |
| ANT-235 | 233 | 0.878 | 1.884 | 0.926 | 2.068 | 1.976 |
| ANT-236 | 234 | 0.678 | 1.457 | 0.902 | 2.013 | 1.735 |
| ANT-373 | 235 | 0.813 | 2.151 | 0.872 | 2.494 | 2.322 |
| ANT-374 | 236 | 0.351 | 1.195 | 0.649 | 2.177 | 1.686 |
| ANT-268 | 237 | 0.713 | 1.707 | 0.775 | 2.192 | 1.950 |
| ANT-237 | 238 | 0.780 | 1.675 | 0.887 | 1.980 | 1.827 |
| ANT-376 | 239 | 0.820 | 2.168 | 0.924 | 2.641 | 2.405 |
| ANT-377 | 240 | 0.246 | 0.651 | 0.852 | 2.434 | 1.543 |
| ANT-270 | 241 | 0.522 | 1.249 | 0.628 | 1.778 | 1.514 |
| ANT-243 | 242 | 0.961 | 2.062 | 1.034 | 2.309 | 2.186 |
| ANT-378 | 243 | 0.901 | 3.064 | 1.070 | 3.590 | 3.327 |
| ANT-244 | 244 | 0.599 | 1.287 | 0.634 | 1.416 | 1.352 |
| ANT-359 | 245 | 0.904 | 2.390 | 1.004 | 2.868 | 2.629 |
| ANT-423 | 247 | 0.966 | 1.874 | 1.051 | 1.995 | 1.934 |
| ANT-424 | 248 | 0.905 | 1.754 | 1.017 | 1.929 | 1.842 |
| ANT-425 | 249 | 0.565 | 1.095 | 0.725 | 1.375 | 1.235 |
| ANT-426 | 250 | 0.248 | 0.480 | 0.408 | 0.774 | 0.627 |
| ANT-427 | 251 | 0.467 | 0.905 | 0.682 | 1.294 | 1.100 |
| ANT-428 | 252 | 0.737 | 1.429 | 0.917 | 1.740 | 1.584 |
| ANT-360 | 254 | 0.302 | 0.412 | 0.691 | 0.976 | 0.833 |
| ANT-271 | 258 | 0.809 | 1.937 | 0.710 | 2.009 | 1.973 |
| ANT-245 | 259 | 1.117 | 2.397 | 0.933 | 2.084 | 2.241 |
| ANT-379 | 260 | 0.904 | 2.391 | 0.939 | 2.683 | 2.537 |
| ANT-380 | 261 | 0.790 | 2.089 | 0.909 | 2.597 | 2.343 |
| ANT-272 | 262 | 0.724 | 1.734 | 0.783 | 2.216 | 1.975 |
| ANT-246 | 263 | 0.659 | 1.578 | 0.763 | 2.159 | 1.868 |
| ANT-381 | 264 | 0.501 | 1.705 | 0.570 | 1.914 | 1.810 |
| ANT-247 | 265 | 0.805 | 1.927 | 0.875 | 2.475 | 2.201 |
| ANT-248 | 266 | 0.846 | 2.026 | 0.837 | 2.368 | 2.197 |
| ANT-382 | 267 | 0.604 | 2.054 | 0.649 | 2.179 | 2.117 |
| ANT-238 | 268 | 0.678 | 1.456 | 0.663 | 1.480 | 1.468 |
| ANT-383 | 269 | 0.912 | 3.102 | 0.985 | 3.307 | 3.205 |
| ANT-273 | 270 | 0.846 | 2.026 | 0.828 | 2.343 | 2.184 |
| ANT-361 | 271 | 0.915 | 2.420 | 0.887 | 2.535 | 2.477 |
| ANT-362 | 272 | 0.975 | 2.580 | 1.039 | 2.970 | 2.775 |
| ANT-274 | 273 | 0.910 | 2.327 | 1.040 | 2.664 | 2.496 |
| ANT-239 | 274 | 0.973 | 2.089 | 0.835 | 1.863 | 1.976 |
| ANT-363 | 275 | 0.458 | 1.211 | 0.459 | 1.311 | 1.261 |
| ANT-364 | 284 | 0.532 | 1.407 | 0.587 | 1.679 | 1.543 |
| ANT-275 | 285 | 0.720 | 1.724 | 0.784 | 2.218 | 1.971 |
| ANT-276 | 286 | 0.944 | 2.260 | 1.011 | 2.860 | 2.560 |
| ANT-384 | 287 | 0.959 | 3.261 | 0.920 | 3.087 | 3.174 |
| ANT-278 | 288 | 0.947 | 2.268 | 0.961 | 2.720 | 2.494 |
| ANT-394 | 289 | 1.039 | 3.534 | 1.004 | 3.370 | 3.452 |
| ANT-294 | 290 | 0.534 | 1.365 | 0.567 | 1.452 | 1.408 |
| ANT-307 | 291 | 0.814 | 2.003 | 0.953 | 2.287 | 2.145 |
| ANT-308 | 292 | 0.458 | 1.127 | 0.515 | 1.237 | 1.182 |
| ANT-295 | 293 | 1.051 | 2.686 | 0.985 | 2.525 | 2.605 |
| ANT-290 | 294 | 1.003 | 2.564 | 1.014 | 2.598 | 2.581 |
| ANT-395 | 295 | 1.077 | 3.661 | 0.884 | 2.968 | 3.315 |
| ANT-396 | 296 | 0.479 | 1.627 | 0.402 | 1.351 | 1.489 |
| ANT-291 | 297 | 0.429 | 1.097 | 0.410 | 1.050 | 1.074 |
| ANT-397 | 298 | 0.995 | 3.384 | 0.997 | 3.345 | 3.365 |
| ANT-398 | 299 | 1.025 | 1.968 | 0.894 | 1.978 | 1.973 |
| ANT-282 | 300 | 0.427 | 1.024 | 0.479 | 1.354 | 1.189 |
| ANT-365 | 301 | 0.888 | 2.348 | 0.880 | 2.514 | 2.431 |
| ANT-366 | 302 | 0.566 | 0.561 | 1.295 | 1.327 | 1.311 |
| ANT-283 | 303 | 0.848 | 2.031 | 0.950 | 2.686 | 2.358 |
| ANT-367 | 304 | 0.860 | 2.276 | 0.842 | 2.405 | 2.340 |
| ANT-429 | 306 | 0.794 | 1.539 | 0.826 | 1.567 | 1.553 |
| ANT-430 | 307 | 0.400 | 0.776 | 0.407 | 0.773 | 0.774 |
| ANT-431 | 308 | 0.210 | 0.407 | 0.230 | 0.436 | 0.422 |
| ANT-296 | 309 | 0.198 | 0.425 | 0.179 | 0.401 | 0.413 |
| ANT-432 | 310 | 0.567 | 1.099 | 0.585 | 1.109 | 1.104 |
| ANT-433 | 311 | 0.803 | 1.557 | 0.873 | 1.656 | 1.606 |
| ANT-434 | 312 | 0.778 | 1.508 | 0.812 | 1.540 | 1.524 |
| ANT-368 | 313 | 0.896 | 2.369 | 0.958 | 2.737 | 2.553 |
| ANT-399 | 314 | 0.906 | 1.739 | 0.837 | 1.852 | 1.795 |
| ANT-297 | 315 | 0.670 | 1.438 | 0.758 | 1.692 | 1.565 |
| ANT-400 | 316 | 0.495 | 0.950 | 0.489 | 1.081 | 1.015 |
| ANT-298 | 317 | 0.957 | 2.446 | 0.984 | 2.521 | 2.484 |
| ANT-401 | 318 | 0.826 | 1.587 | 0.774 | 1.711 | 1.649 |
| ANT-402 | 319 | 0.608 | 1.168 | 0.534 | 1.181 | 1.174 |
| ANT-299 | 320 | 0.773 | 1.660 | 0.806 | 1.800 | 1.730 |
| ANT-300 | 321 | 0.965 | 2.072 | 1.064 | 2.376 | 2.224 |
| ANT-403 | 322 | 1.069 | 2.052 | 1.021 | 2.258 | 2.155 |
| ANT-404 | 323 | 0.957 | 1.838 | 0.837 | 1.852 | 1.845 |
| ANT-301 | 324 | 0.975 | 2.492 | 0.802 | 2.054 | 2.273 |
| ANT-309 | 325 | 0.927 | 2.281 | 0.887 | 2.129 | 2.205 |
| ANT-310 | 326 | 0.920 | 2.263 | 0.968 | 2.324 | 2.293 |
| ANT-311 | 327 | 0.870 | 2.141 | 0.875 | 2.100 | 2.120 |
| ANT-312 | 328 | 0.766 | 1.885 | 0.789 | 1.893 | 1.889 |
| ANT-313 | 329 | 0.540 | 1.330 | 0.535 | 1.285 | 1.307 |
| ANT-314 | 330 | 0.579 | 1.425 | 0.600 | 1.439 | 1.432 |
| ANT-315 | 331 | 0.634 | 1.560 | 0.683 | 1.639 | 1.599 |
| ANT-316 | 332 | 0.817 | 2.011 | 0.787 | 1.889 | 1.950 |
| ANT-249 | 333 | 0.672 | 1.610 | 0.594 | 1.680 | 1.645 |
| ANT-435 | 334 | 0.579 | 1.123 | 0.693 | 1.315 | 1.219 |
| ANT-284 | 335 | 0.442 | 1.058 | 0.463 | 1.311 | 1.185 |
| ANT-436 | 336 | 0.566 | 1.097 | 0.541 | 1.026 | 1.061 |
| ANT-437 | 337 | 0.479 | 0.929 | 0.463 | 0.878 | 0.904 |
| ANT-438 | 338 | 0.647 | 1.255 | 0.632 | 1.199 | 1.227 |
| ANT-439 | 339 | 0.647 | 1.255 | 0.618 | 1.172 | 1.213 |
| ANT-317 | 340 | 0.691 | 1.700 | 0.699 | 1.677 | 1.689 |
| ANT-318 | 341 | 0.896 | 2.205 | 0.799 | 1.919 | 2.062 |
| ANT-319 | 342 | 0.723 | 1.779 | 0.744 | 1.786 | 1.783 |
| ANT-320 | 345 | 0.520 | 1.280 | 0.605 | 1.451 | 1.366 |
| ANT-321 | 346 | 0.393 | 0.968 | 0.383 | 0.918 | 0.943 |
| ANT-250 | 347 | 0.548 | 1.312 | 0.534 | 1.510 | 1.411 |
| ANT-322 | 348 | 0.503 | 1.239 | 0.567 | 1.362 | 1.300 |
| ANT-405 | 349 | 0.621 | 1.193 | 0.540 | 1.194 | 1.193 |
| ANT-406 | 350 | 0.996 | 1.913 | 0.901 | 1.993 | 1.953 |
| ANT-302 | 351 | 0.903 | 1.938 | 0.976 | 2.180 | 2.059 |
| ANT-251 | 352 | 0.793 | 1.898 | 0.819 | 2.316 | 2.107 |
| ANT-323 | 353 | 0.408 | 1.004 | 0.430 | 1.033 | 1.018 |
| ANT-440 | 354 | 0.381 | 0.738 | 0.386 | 0.733 | 0.736 |
| ANT-441 | 355 | 0.350 | 0.680 | 0.381 | 0.723 | 0.701 |
| ANT-324 | 356 | 0.238 | 0.586 | 0.289 | 0.695 | 0.640 |
| ANT-325 | 357 | 0.593 | 1.459 | 0.635 | 1.525 | 1.492 |
| ANT-442 | 358 | 0.695 | 1.347 | 0.732 | 1.389 | 1.368 |
| ANT-443 | 359 | 0.581 | 1.127 | 0.643 | 1.219 | 1.173 |
| ANT-326 | 361 | 0.379 | 0.933 | 0.412 | 0.989 | 0.961 |
| ANT-327 | 362 | 0.402 | 0.989 | 0.555 | 1.333 | 1.161 |
| ANT-328 | 363 | 0.411 | 1.012 | 0.462 | 1.109 | 1.060 |
| ANT-329 | 364 | 0.345 | 0.849 | 0.353 | 0.848 | 0.848 |
| ANT-445 | 365 | 0.630 | 1.222 | 0.692 | 1.314 | 1.268 |
| ANT-252 | 366 | 0.749 | 1.794 | 0.733 | 2.072 | 1.933 |
| ANT-446 | 367 | 0.569 | 1.103 | 0.589 | 1.118 | 1.111 |
| ANT-447 | 368 | 0.649 | 1.258 | 0.680 | 1.290 | 1.274 |
| ANT-448 | 369 | 0.844 | 1.637 | 0.849 | 1.611 | 1.624 |
| ANT-285 | 370 | 0.511 | 1.223 | 0.571 | 1.616 | 1.420 |
| ANT-330 | 37 | 0.341 | 0.838 | 0.428 | 1.027 | 0.933 |
| ANT-331 | 372 | 0.779 | 1.916 | 0.826 | 1.983 | 1.949 |
| ANT-332 | 373 | 0.763 | 1.877 | 0.665 | 1.596 | 1.737 |
| ANT-333 | 374 | 0.774 | 1.904 | 0.786 | 1.886 | 1.895 |
| ANT-334 | 376 | 0.510 | 1.255 | 0.493 | 1.184 | 1.219 |
| ANT-335 | 378 | 0.918 | 2.259 | 1.005 | 2.414 | 2.336 |
| ANT-336 | 379 | 0.910 | 2.238 | 0.997 | 2.393 | 2.316 |
| ANT-253 | 380 | 0.616 | 1.476 | 0.656 | 1.855 | 1.666 |
| ANT-407 | 381 | 0.297 | 0.571 | 0.284 | 0.629 | 0.600 |
| ANT-408 | 384 | 0.506 | 0.971 | 0.424 | 0.939 | 0.955 |
| ANT-338 | 385 | 0.656 | 1.613 | 0.764 | 1.834 | 1.724 |
| ANT-339 | 386 | 0.562 | 1.383 | 0.556 | 1.335 | 1.359 |
| ANT-340 | 387 | 0.382 | 0.941 | 0.394 | 0.946 | 0.944 |
| ANT-255 | 388 | 0.828 | 1.984 | 0.849 | 2.402 | 2.193 |
| ANT-256 | 389 | 0.939 | 2.248 | 0.920 | 2.602 | 2.425 |
| ANT-257 | 390 | 0.718 | 1.719 | 0.616 | 1.742 | 1.731 |
| ANT-258 | 39 | 0.896 | 2.145 | 1.053 | 2.979 | 2.562 |
| ANT-369 | 392 | 0.681 | 1.802 | 0.701 | 2.002 | 1.902 |
| ANT-449 | 393 | 0.576 | 1.525 | 0.550 | 1.573 | 1.549 |
| ANT-450 | 394 | 0.635 | 1.681 | 0.550 | 1.572 | 1.626 |
| ANT-451 | 395 | 0.743 | 1.966 | 0.692 | 1.977 | 1.972 |
| ANT-286 | 396 | 0.191 | 0.489 | 0.162 | 0.416 | 0.453 |
| ANT-452 | 397 | 0.262 | 0.693 | 0.239 | 0.682 | 0.688 |
| ANT-455 | 400 | 0.806 | 2.133 | 0.755 | 2.158 | 2.145 |
| ANT-341 | 401 | 0.839 | 2.064 | 0.891 | 2.140 | 2.102 |
| ANT-342 | 402 | 0.396 | 0.974 | 0.397 | 0.952 | 0.963 |
| ANT-456 | 403 | 0.428 | 1.133 | 0.383 | 1.095 | 1.114 |
| ANT-457 | 404 | 0.539 | 1.425 | 0.501 | 1.432 | 1.429 |
| ANT-458 | 405 | 0.452 | 1.197 | 0.397 | 1.136 | 1.166 |
| ANT-370 | 406 | 0.736 | 1.946 | 0.705 | 2.015 | 1.981 |
| ANT-287 | 407 | 0.544 | 1.392 | 0.457 | 1.171 | 1.282 |
| ANT-409 | 408 | 0.977 | 1.877 | 1.034 | 2.286 | 2.082 |
| ANT-288 | 409 | 1.032 | 2.637 | 1.030 | 2.638 | 2.638 |
| ANT-371 | 410 | 1.065 | 2.817 | 1.062 | 3.036 | 2.927 |
| ANT-372 | 418 | 0.994 | 2.629 | 0.986 | 2.817 | 2.723 |
| ANT-289 | 419 | 0.722 | 1.846 | 0.596 | 1.528 | 1.687 |

TABLE 13-1
Human TDP-43 gene RNA expression level, etc. in A204 cell treated with respective
gapmers (1.5 μM)

| | | | Ratio of | | Ratio of | Average of |
|---|---|---|---|---|---|---|
| | | mRNA | mRNA | mRNA | mRNA | Ratio of |
| | | expression | expression | expression | expression | mRNA |
| | | level | level/ANT- | level | level/ANT- | expression |
| ASO name | SEQ ID NO: | (exon2-3) | 10(exon2-3) | (3′UTR) | 10(3′UTR) | level |
| vehicle | — | 1.000 | 2.598 | 1.000 | 2.630 | 2.61 |
| ANT-10 | 114 | 0.385 | 1.000 | 0.380 | 1.000 | 1.00 |
| ANT-444 | 360 | 0.817 | 2.179 | 0.773 | 2.452 | 2.316 |
| ANT-453 | 398 | 0.305 | 0.813 | 0.266 | 0.843 | 0.828 |
| ANT-454 | 399 | 0.354 | 0.943 | 0.298 | 0.945 | 0.944 |
| ANT-459 | 412 | 0.950 | 2.467 | 0.937 | 2.465 | 2.466 |
| ANT-460 | 413 | 0.898 | 2.332 | 0.979 | 2.575 | 2.454 |
| ANT-461 | 414 | 0.517 | 1.343 | 0.467 | 1.229 | 1.286 |
| ANT-462 | 415 | 0.672 | 1.745 | 0.782 | 2.056 | 1.900 |
| ANT-463 | 416 | 0.893 | 2.319 | 0.822 | 2.163 | 2.241 |
| ANT-464 | 417 | 0.826 | 2.144 | 0.959 | 2.524 | 2.334 |
| ANT-465 | 277 | 0.477 | 1.238 | 0.661 | 1.738 | 1.488 |
| ANT-466 | 278 | 0.502 | 1.305 | 0.434 | 1.140 | 1.222 |
| ANT-467 | 279 | 0.540 | 1.404 | 0.484 | 1.272 | 1.338 |
| ANT-468 | 280 | 0.452 | 1.175 | 0.453 | 1.192 | 1.183 |
| ANT-469 | 281 | 0.552 | 1.433 | 0.537 | 1.412 | 1.422 |
| ANT-470 | 282 | 0.655 | 1.701 | 0.621 | 1.633 | 1.667 |
| ANT-471 | 253 | 0.588 | 1.528 | 0.661 | 1.738 | 1.633 |
| ANT-472 | 255 | 0.328 | 0.851 | 0.481 | 1.265 | 1.058 |
| ANT-473 | 256 | 0.763 | 2.035 | 0.725 | 2.299 | 2.167 |
| ANT-474 | 257 | 0.867 | 2.312 | 0.788 | 2.500 | 2.406 |
| ANT-476 | 246 | 0.911 | 2.429 | 0.808 | 2.563 | 2.496 |
| ANT-477 | 276 | 0.650 | 1.732 | 0.562 | 1.782 | 1.757 |
| ANT-478 | 283 | 0.587 | 1.565 | 0.570 | 1.808 | 1.687 |
| ANT-479 | 305 | 0.740 | 1.974 | 0.715 | 2.268 | 2.121 |
| ANT-480 | 343 | 0.380 | 1.013 | 0.329 | 1.044 | 1.029 |
| ANT-481 | 344 | 0.502 | 1.339 | 0.484 | 1.535 | 1.437 |
| ANT-482 | 375 | 0.439 | 1.170 | 0.367 | 1.163 | 1.167 |
| ANT-483 | 377 | 0.386 | 1.030 | 0.358 | 1.137 | 1.083 |
| ANT-484 | 382 | 0.431 | 1.149 | 0.365 | 1.158 | 1.154 |
| ANT-485 | 383 | 0.844 | 2.250 | 0.741 | 2.350 | 2.300 |
| ANT-486 | 411 | 0.961 | 2.564 | 0.891 | 2.826 | 2.695 |

TABLE 14-1
Human TDP-43 gene RNA expression level, etc. in A204 cell treated with respective
gapmers (1.5 μM)

| | | | Ratio of | | Ratio of | Average of |
|---|---|---|---|---|---|---|
| | | mRNA | mRNA | mRNA | mRNA | ratio of |
| | | expression | expression | expression | expression | mRNA |
| | | level | level/ANT- | level | level/ANT- | expression |
| ASO name | SEQ ID NO: | (exon2-3) | 10(exon2-3) | (3′UTR) | 10(3′UTR) | level |
| vehicle | — | 1 | 2.556 | 1 | 2.562 | 2.559 |
| ANT-10 | 114 | 0.391 | 1.000 | 0.390 | 1.000 | 1.000 |
| ANT-232 | 475 | 0.982 | 2.108 | 0.942 | 2.102 | 2.105 |
| ANT-267 | 476 | 0.800 | 1.915 | 0.819 | 2.316 | 2.116 |
| ANT-375 | 477 | 0.912 | 3.103 | 0.978 | 3.283 | 3.283 |
| ANT-269 | 478 | 0.468 | 1.121 | 0.531 | 1.502 | 1.311 |
| ANT-303 | 479 | 0.857 | 2.108 | 0.931 | 2.234 | 2.171 |
| ANT-304 | 480 | 0.822 | 2.022 | 0.910 | 2.185 | 2.103 |
| ANT-305 | 481 | 0.890 | 2.191 | 1.100 | 2.640 | 2.415 |
| ANT-306 | 482 | 0.446 | 1.098 | 0.520 | 1.249 | 1.174 |
| ANT-277 | 483 | 0.603 | 1.444 | 0.666 | 1.883 | 1.664 |
| ANT-279 | 484 | 0.819 | 1.961 | 0.926 | 2.621 | 2.291 |
| ANT-240 | 485 | 0.792 | 1.701 | 0.730 | 1.630 | 1.665 |
| ANT-385 | 486 | 0.465 | 1.580 | 0.531 | 1.780 | 1.680 |
| ANT-280 | 487 | 0.402 | 0.962 | 0.451 | 1.276 | 1.119 |
| ANT-386 | 488 | 0.637 | 2.168 | 0.650 | 2.182 | 2.175 |
| ANT-387 | 489 | 0.340 | 1.158 | 0.353 | 1.186 | 1.172 |
| ANT-281 | 490 | 0.443 | 1.061 | 0.445 | 1.259 | 1.160 |
| ANT-388 | 491 | 0.513 | 1.743 | 0.504 | 1.690 | 1.716 |
| ANT-241 | 492 | 0.810 | 1.740 | 0.956 | 2.133 | 1.937 |
| ANT-389 | 493 | 0.381 | 1.296 | 0.387 | 1.299 | 1.297 |
| ANT-390 | 494 | 0.567 | 1.929 | 0.592 | 1.985 | 1.957 |
| ANT-242 | 495 | 0.632 | 1.356 | 0.645 | 1.441 | 1.399 |
| ANT-391 | 496 | 0.625 | 2.125 | 0.588 | 1.973 | 2.049 |
| ANT-292 | 497 | 0.852 | 2.178 | 0.782 | 2.003 | 2.090 |
| ANT-392 | 498 | 0.918 | 3.121 | 0.887 | 2.978 | 3.049 |
| ANT-393 | 499 | 0.568 | 1.931 | 0.496 | 1.664 | 1.798 |
| ANT-293 | 500 | 0.789 | 1.694 | 0.912 | 2.036 | 1.865 |
| ANT-337 | 501 | 0.734 | 1.807 | 0.709 | 1.701 | 1.754 |
| ANT-254 | 502 | 0.624 | 1.494 | 0.561 | 1.587 | 1.541 |

TABLE 15
Human TDP-43 gene RNA expression level, etc. in A204 cell treated with respective
gapmers (1.5 μM)

| | | | Ratio of | | | Average of |
|---|---|---|---|---|---|---|
| | | mRNA | mRNA | mRNA | Ratio of mRNA | ratio of |
| | | expression | expression | expression | expression | mRNA |
| | SEQ ID | level | level/ANT- | level | level/ANT-266 | expression |
| ASO name | NO: | (exon2-3) | 266(exon2-3) | (exon 5-6) | (exon5-6) | level |
| vehicle | — | 1.000 | 4.612 | 1.000 | 5.902 | 5.257 |
| ANT-263 | 106 | 0.103 | 0.475 | 0.073 | 0.432 | 0.454 |
| ANT-264 | 106 | 0.104 | 0.481 | 0.081 | 0.481 | 0.481 |
| ANT-265 | 114 | 0.147 | 0.679 | 0.112 | 0.660 | 0.669 |
| ANT-266 | 114 | 0.217 | 1.000 | 0.169 | 1.000 | 1.000 |

TABLE 16
Human TDP-43 gene RNA expression level, etc. in A204 cell treated with respective
gapmers (0.6 μM)

| | | | Ratio of RNA | | | |
|---|---|---|---|---|---|---|
| | | RNA | expression | RNA | Ratio of RNA | Average of |
| | | expression | level/ANT- | expression | expression | ratio of RNA |
| | SEQ ID | level | 266 | level | level/ANT- | expression |
| ASO name | NO: | (exon2-3) | (exon2-3) | (3′UTR) | 266(3′UTR) | level |
| vehicle | — | 1.000 | 1.622 | 1.000 | 1.863 | 1.742 |
| ANT-266 | 114 | 0.617 | 1.000 | 0.537 | 1.000 | 1.000 |
| ANT-343 | 427 | 0.693 | 1.125 | 0.625 | 1.164 | 1.144 |
| ANT-344 | 427 | 0.626 | 1.015 | 0.622 | 1.159 | 1.087 |
| ANT-345 | 427 | 0.548 | 0.889 | 0.501 | 0.933 | 0.911 |
| ANT-346 | 427 | 0.469 | 0.760 | 0.428 | 0.798 | 0.779 |
| ANT-347 | 427 | 0.542 | 0.879 | 0.518 | 0.965 | 0.922 |
| ANT-348 | 427 | 0.438 | 0.711 | 0.386 | 0.718 | 0.714 |

TABLE 17
Human TDP-43 gene RNA expression level, etc. in A204 cell treated with respective
gapmers (0.6 μM)

| | | | Ratio of | | | |
|---|---|---|---|---|---|---|
| | | | mRNA | | Ratio of | |
| | | mRNA | expression | | mRNA | Average of |
| | | expression | level/ | mRNA | expression | ratio of mRNA |
| | SEQ ID | level | ANT-266 | expression | level/ANT- | expression |
| ASO name | NO: | (exon2-3) | (exon2-3) | level (3′UTR) | 266(3′UTR) | level |
| vehicle | — | 1.000 | 1.604 | 1.000 | 1.447 | 1.525 |
| ANT-266 | 114 | 0.624 | 1.000 | 0.691 | 1.000 | 1.000 |
| ANT-349 | 44 | 0.827 | 1.534 | 1.037 | 1.725 | 1.629 |
| ANT-350 | 107 | 0.591 | 1.098 | 0.759 | 1.263 | 1.180 |
| ANT-351 | 110 | 0.728 | 1.352 | 0.963 | 1.602 | 1.477 |
| ANT-352 | 112 | 0.464 | 0.862 | 0.578 | 0.962 | 0.912 |
| ANT-353 | 74 | 0.763 | 1.417 | 0.946 | 1.574 | 1.495 |
| ANT-354 | 105 | 0.691 | 1.283 | 0.787 | 1.310 | 1.296 |
| ANT-410 | 396 | 0.233 | 0.432 | 0.255 | 0.425 | 0.428 |
| ANT-411 | 356 | 0.350 | 0.650 | 0.418 | 0.695 | 0.673 |
| ANT-475 | 364 | 0.406 | 0.754 | 0.546 | 0.908 | 0.831 |
| ANT-487 | 364 | 0.410 | 0.760 | 0.517 | 0.861 | 0.810 |
| ANT-488 | 364 | 0.480 | 0.891 | 0.626 | 1.041 | 0.966 |
| ANT-489 | 112 | 0.672 | 1.247 | 0.964 | 1.603 | 1.425 |
| ANT-490 | 112 | 0.711 | 1.319 | 0.936 | 1.557 | 1.438 |
| ANT-491 | 25 | 0.491 | 0.911 | 0.953 | 1.586 | 1.248 |
| ANT-492 | 25 | 0.489 | 0.907 | 0.906 | 1.508 | 1.207 |
| ANT-493 | 25 | 0.459 | 0.853 | 0.908 | 1.510 | 1.181 |
| ANT-494 | 42 | 0.268 | 0.497 | 0.326 | 0.543 | 0.520 |
| ANT-495 | 42 | 0.256 | 0.475 | 0.306 | 0.509 | 0.492 |
| ANT-496 | 42 | 0.259 | 0.480 | 0.319 | 0.531 | 0.506 |
| ANT-497 | 309 | 0.320 | 0.514 | 0.291 | 0.421 | 0.468 |
| ANT-498 | 309 | 0.409 | 0.657 | 0.335 | 0.484 | 0.570 |
| ANT-499 | 309 | 0.425 | 0.682 | 0.408 | 0.591 | 0.636 |
| ANT-500 | 402 | 0.451 | 0.724 | 0.428 | 0.620 | 0.672 |
| ANT-501 | 402 | 0.550 | 0.883 | 0.559 | 0.808 | 0.846 |
| ANT-502 | 402 | 0.507 | 0.814 | 0.490 | 0.709 | 0.762 |

TABLE 18-1
Human TDP-43 gene RNA expression level, etc. in A204 cell treated with respective
gapmers (0.6 μM)

| | | | Ratio of | | Ratio of | Average of |
|---|---|---|---|---|---|---|
| | | mRNA | mRNA | mRNA | mRNA | ratio of |
| | | expression | expression | expression | expression | mRNA |
| | SEQ ID | level | level/ANT- | level | level/ANT- | expression |
| ASO name | NO: | (exon2-3) | 266(exon2-3) | (3′UTR) | 266(3′UTR) | level |
| vehicle | — | 1.000 | 1.586 | 1.000 | 1.597 | 1.591 |
| ANT-266 | 114 | 0.631 | 1.000 | 0.626 | 1.000 | 1.000 |
| ANT-503 | 250 | 0.160 | 0.254 | 0.273 | 0.436 | 0.345 |
| ANT-504 | 250 | 0.453 | 0.726 | 0.709 | 1.026 | 0.876 |
| ANT-505 | 250 | 0.297 | 0.471 | 0.515 | 0.822 | 0.647 |
| ANT-506 | 308 | 0.258 | 0.414 | 0.251 | 0.363 | 0.388 |
| ANT-507 | 308 | 0.217 | 0.349 | 0.207 | 0.299 | 0.324 |
| ANT-508 | 308 | 0.194 | 0.308 | 0.196 | 0.312 | 0.310 |
| ANT-509 | 112 | 0.828 | 1.314 | 0.882 | 1.408 | 1.361 |
| ANT-510 | 112 | 0.874 | 1.386 | 0.954 | 1.522 | 1.454 |
| ANT-511 | 112 | 0.858 | 1.360 | 0.812 | 1.296 | 1.328 |
| ANT-512 | 254 | 0.829 | 1.315 | 0.786 | 1.255 | 1.285 |
| ANT-513 | 131 | 0.641 | 1.017 | 0.578 | 0.923 | 0.970 |
| ANT-514 | 307 | 0.322 | 0.511 | 0.279 | 0.445 | 0.478 |
| ANT-515 | 112 | 0.867 | 1.375 | 0.946 | 1.511 | 1.443 |
| ANT-516 | 356 | 0.338 | 0.536 | 0.348 | 0.556 | 0.546 |
| ANT-517 | 356 | 0.472 | 0.748 | 0.419 | 0.669 | 0.709 |
| ANT-518 | 251 | 0.418 | 0.934 | 0.712 | 1.435 | 1.184 |
| ANT-519 | 251 | 0.529 | 1.183 | 0.805 | 1.622 | 1.402 |
| ANT-520 | 251 | 0.439 | 0.982 | 0.707 | 1.425 | 1.203 |
| ANT-521 | 254 | 0.523 | 1.169 | 0.702 | 1.414 | 1.291 |
| ANT-522 | 254 | 0.476 | 1.063 | 0.656 | 1.321 | 1.192 |
| ANT-523 | 354 | 0.352 | 0.787 | 0.384 | 0.774 | 0.781 |
| ANT-524 | 80 | 0.430 | 0.960 | 0.464 | 0.935 | 0.947 |
| ANT-525 | 80 | 0.545 | 1.218 | 0.635 | 1.279 | 1.249 |
| ANT-526 | 80 | 0.534 | 1.192 | 0.620 | 1.249 | 1.221 |
| ANT-527 | 307 | 0.717 | 1.602 | 0.772 | 1.556 | 1.579 |
| ANT-528 | 307 | 0.681 | 1.521 | 0.779 | 1.569 | 1.545 |
| ANT-529 | 355 | 0.367 | 0.821 | 0.383 | 0.771 | 0.796 |

The calculated values and the measured values of ESI-TOF-MS of the respective gapmers obtained in Examples are as follows.

TABLE 19

| ASO | Calculated | Measured |
|---|---|---|
| name | value | value |
| ANT-1 | 7194.111 | 7194.116 |
| ANT-2 | 7150.097 | 7150.113 |
| ANT-3 | 7207.059 | 7207.067 |
| ANT-4 | 7216.070 | 7216.072 |
| ANT-5 | 7283.062 | 7283.071 |
| ANT-6 | 7187.068 | 7187.078 |
| ANT-7 | 7302.069 | 7302.086 |
| ANT-8 | 7209.062 | 7209.073 |
| ANT-9 | 7183.071 | 7183.079 |
| ANT-10 | 7174.120 | 7174.129 |
| ANT-11 | 7132.074 | 7132.082 |
| ANT-12 | 7152.126 | 7152.131 |
| ANT-13 | 7176.088 | 7176.089 |
| ANT-14 | 7238.125 | 7238.129 |
| ANT-15 | 7174.085 | 7174.083 |
| ANT-16 | 7193.092 | 7193.085 |
| ANT-17 | 7180.024 | 7180.032 |
| ANT-18 | 7163.045 | 7163.050 |
| ANT-19 | 7218.038 | 7218.048 |
| ANT-20 | 7239.049 | 7239.059 |
| ANT-21 | 7243.080 | 7243.094 |
| ANT-22 | 7149.113 | 7149.114 |
| ANT-23 | 7207.154 | 7207.156 |
| ANT-24 | 7311.116 | 7311.119 |
| ANT-25 | 7293.093 | 7293.099 |
| ANT-26 | 7149.053 | 7149.038 |
| ANT-30 | 7146.101 | 7146.106 |
| ANT-31 | 7304.072 | 7304.071 |
| ANT-32 | 7225.117 | 7225.136 |
| ANT-33 | 7244.064 | 7244.083 |
| ANT-34 | 7159.109 | 7159.125 |
| ANT-35 | 7166.092 | 7166.108 |
| ANT-36 | 7288.078 | 7288.088 |
| ANT-37 | 7170.028 | 7170.043 |
| ANT-38 | 7149.113 | 7149.120 |
| ANT-39 | 7214.042 | 7214.057 |
| ANT-40 | 7111.988 | 7111.976 |
| ANT-41 | 7219.057 | 7219.047 |
| ANT-42 | 7099.040 | 7099.028 |
| ANT-43 | 7231.021 | 7231.038 |
| ANT-44 | 7220.102 | 7220.077 |
| ANT-45 | 7209.002 | 7208.983 |
| ANT-46 | 7172.057 | 7172.036 |
| ANT-47 | 7163.105 | 7163.086 |
| ANT-48 | 7169.044 | 7169.028 |
| ANT-49 | 7167.076 | 7167.052 |
| ANT-50 | 7242.121 | 7242.104 |
| ANT-51 | 7135.026 | 7134.955 |
| ANT-52 | 7195.035 | 7195.020 |
| ANT-53 | 7125.091 | 7125.076 |
| ANT-54 | 7249.044 | 7249.035 |
| ANT-55 | 7176.028 | 7176.013 |
| ANT-56 | 7169.044 | 7169.031 |
| ANT-57 | 7183.132 | 7183.114 |
| ANT-58 | 7132.074 | 7132.062 |
| ANT-59 | 7271.099 | 7271.073 |
| ANT-60 | 7123.123 | 7123.091 |
| ANT-61 | 7341.043 | 7341.020 |
| ANT-62 | 7255.104 | 7255.092 |
| ANT-63 | 7171.073 | 7171.058 |
| ANT-64 | 7210.106 | 7210.086 |
| ANT-67 | 7253.015 | 7253.017 |
| ANT-68 | 7110.020 | 7110.006 |
| ANT-69 | 7144.098 | 7144.082 |
| ANT-70 | 7175.104 | 7175.089 |
| ANT-71 | 7134.103 | 7134.083 |
| ANT-72 | 7121.155 | 7121.152 |
| ANT-73 | 7145.022 | 7145.021 |
| ANT-74 | 7133.119 | 7133.116 |
| ANT-75 | 7221.086 | 7221.081 |
| ANT-76 | 7284.081 | 7284.074 |
| ANT-77 | 7216.045 | 7216.033 |
| ANT-78 | 7142.130 | 7142.119 |
| ANT-79 | 7242.036 | 7242.022 |
| ANT-80 | 7219.057 | 7219.049 |
| ANT-81 | 7244.004 | 7243.988 |
| ANT-82 | 7206.050 | 7206.030 |
| ANT-83 | 7222.070 | 7222.054 |
| ANT-84 | 7215.086 | 7215.083 |
| ANT-85 | 7149.113 | 7149.119 |
| ANT-86 | 7244.064 | 7244.058 |
| ANT-87 | 7133.119 | 7133.100 |
| ANT-88 | 7211.090 | 7211.087 |
| ANT-89 | 7219.118 | 7219.112 |
| ANT-90 | 7134.103 | 7134.094 |
| ANT-91 | 7117.124 | 7117.105 |
| ANT-92 | 7191.064 | 7191.040 |
| ANT-93 | 7172.057 | 7172.047 |
| ANT-94 | 7165.013 | 7164.991 |
| ANT-95 | 7175.069 | 7175.048 |
| ANT-96 | 7170.089 | 7170.065 |
| ANT-97 | 7235.077 | 7235.060 |
| ANT-98 | 7119.031 | 7119.225 |
| ANT-99 | 7140.067 | 7140.068 |
| ANT-100 | 7138.039 | 7138.036 |
| ANT-101 | 7135.087 | 7135.104 |
| ANT-102 | 7213.118 | 7213.120 |
| ANT-103 | 7238.988 | 7238.974 |
| ANT-104 | 7232.065 | 7232.061 |
| ANT-105 | 7242.061 | 7242.047 |
| ANT-106 | 7206.075 | 7206.067 |
| ANT-107 | 7195.035 | 7195.025 |
| ANT-108 | 7234.033 | 7234.019 |
| ANT-109 | 7267.067 | 7267.057 |
| ANT-110 | 7267.067 | 7267.056 |
| ANT-111 | 7234.033 | 7234.049 |
| ANT-112 | 7176.088 | 7176.103 |
| ANT-113 | 7209.147 | 7209.132 |
| ANT-117 | 7258.056 | 7258.039 |
| ANT-118 | 7162.061 | 7162.049 |
| ANT-119 | 7171.073 | 7171.061 |
| ANT-120 | 7189.036 | 7189.021 |
| ANT-121 | 7292.074 | 7292.054 |
| ANT-122 | 7255.043 | 7255.017 |
| ANT-123 | 7253.075 | 7253.061 |
| ANT-124 | 7229.113 | 7229.090 |
| ANT-125 | 7302.069 | 7302.052 |
| ANT-126 | 7202.078 | 7202.063 |
| ANT-127 | 7167.076 | 7167.061 |
| ANT-128 | 7210.046 | 7210.032 |
| ANT-129 | 7179.100 | 7179.080 |
| ANT-130 | 7174.120 | 7174.107 |
| ANT-131 | 7142.070 | 7142.052 |
| ANT-132 | 7138.074 | 7138.055 |
| ANT-133 | 7151.081 | 7151.063 |
| ANT-134 | 7199.091 | 7199.069 |
| ANT-135 | 7247.076 | 7247.084 |
| ANT-136 | 7132.135 | 7132.146 |
| ANT-137 | 7159.049 | 7159.077 |
| ANT-138 | 7166.057 | 7166.075 |
| ANT-139 | 7246.032 | 7246.057 |
| ANT-140 | 7181.068 | 7181.081 |
| ANT-141 | 7182.112 | 7182.113 |
| ANT-142 | 7205.031 | 7205.002 |
| ANT-143 | 7146.066 | 7146.043 |
| ANT-144 | 7375.061 | 7375.029 |
| ANT-145 | 7212.074 | 7212.087 |
| ANT-146 | 7231.141 | 7231.158 |
| ANT-147 | 7170.089 | 7170.107 |
| ANT-148 | 7152.066 | 7152.093 |
| ANT-149 | 7157.081 | 7157.108 |
| ANT-150 | 7202.139 | 7202.157 |
| ANT-151 | 7168.060 | 7168.062 |
| ANT-152 | 7252.091 | 7252.105 |
| ANT-153 | 7117.063 | 7117.081 |
| ANT-154 | 7248.060 | 7248.081 |
| ANT-155 | 7195.095 | 7195.106 |
| ANT-156 | 7238.125 | 7238.142 |
| ANT-157 | 7190.080 | 7190.089 |
| ANT-158 | 7166.057 | 7166.073 |
| ANT-159 | 7149.018 | 7149.033 |
| ANT-160 | 7274.111 | 7274.125 |
| ANT-161 | 7170.028 | 7170.030 |
| ANT-162 | 7251.072 | 7251.090 |
| ANT-163 | 7231.081 | 7231.100 |
| ANT-164 | 7197.063 | 7197.076 |
| ANT-165 | 7201.059 | 7201.074 |
| ANT-166 | 7191.064 | 7191.076 |
| ANT-167 | 7205.031 | 7205.046 |
| ANT-168 | 7289.062 | 7289.070 |
| ANT-169 | 7213.058 | 7213.072 |
| ANT-170 | 7240.093 | 7240.108 |
| ANT-171 | 7169.070 | 7169.077 |
| ANT-172 | 7200.075 | 7200.083 |
| ANT-173 | 7218.073 | 7218.086 |
| ANT-174 | 7195.035 | 7195.047 |
| ANT-175 | 7290.106 | 7290.127 |
| ANT-176 | 7203.123 | 7203.137 |
| ANT-177 | 7240.093 | 7240.104 |
| ANT-178 | 7213.058 | 7213.073 |
| ANT-179 | 7224.038 | 7224.047 |
| ANT-180 | 7124.107 | 7124.115 |
| ANT-181 | 7184.080 | 7184.083 |
| ANT-182 | 7214.042 | 7214.050 |
| ANT-183 | 7144.038 | 7144.042 |
| ANT-184 | 7240.093 | 7240.104 |
| ANT-185 | 7128.043 | 7128.048 |
| ANT-186 | 7083.045 | 7083.048 |
| ANT-187 | 7265.039 | 7265.037 |
| ANT-188 | 7204.046 | 7204.054 |
| ANT-189 | 7136.071 | 7136.072 |
| ANT-190 | 7177.132 | 7177.140 |
| ANT-191 | 7143.114 | 7143.121 |
| ANT-192 | 7181.103 | 7181.109 |
| ANT-193 | 7162.061 | 7162.073 |
| ANT-194 | 7190.020 | 7190.019 |
| ANT-195 | 7298.013 | 7298.021 |
| ANT-196 | 7147.050 | 7147.053 |
| ANT-197 | 7217.054 | 7217.058 |
| ANT-198 | 7210.071 | 7210.066 |
| ANT-199 | 7195.095 | 7195.102 |
| ANT-200 | 7145.082 | 7145.078 |
| ANT-201 | 7165.134 | 7165.132 |
| ANT-202 | 7291.030 | 7291.041 |
| ANT-203 | 7131.055 | 7131.056 |
| ANT-204 | 7216.070 | 7216.074 |
| ANT-205 | 7174.025 | 7174.025 |
| ANT-206 | 7258.056 | 7258.057 |
| ANT-207 | 7267.067 | 7267.066 |
| ANT-208 | 7196.079 | 7196.090 |
| ANT-209 | 7202.104 | 7202.104 |
| ANT-210 | 7250.088 | 7250.098 |
| ANT-211 | 7265.039 | 7265.044 |
| ANT-212 | 7251.072 | 7251.079 |
| ANT-213 | 7265.039 | 7265.052 |
| ANT-214 | 7185.039 | 7185.049 |
| ANT-215 | 7177.072 | 7177.080 |
| ANT-216 | 7263.011 | 7263.018 |
| ANT-217 | 7225.057 | 7225.068 |
| ANT-218 | 7187.068 | 7187.083 |
| ANT-219 | 7266.083 | 7266.085 |
| ANT-220 | 7207.059 | 7207.067 |
| ANT-221 | 7196.079 | 7196.094 |
| ANT-222 | 7214.102 | 7214.117 |
| ANT-223 | 7191.064 | 7191.076 |
| ANT-224 | 7171.073 | 7171.090 |
| ANT-225 | 7207.059 | 7207.064 |
| ANT-226 | 7165.013 | 7165.030 |
| ANT-227 | 7174.085 | 7174.096 |
| ANT-228 | 7183.036 | 7183.043 |
| ANT-229 | 7234.033 | 7234.039 |
| ANT-230 | 7174.025 | 7174.035 |
| ANT-231 | 7177.037 | 7177.046 |
| ANT-232 | 7259.075 | 7259.138 |
| ANT-233 | 7134.042 | 7134.056 |
| ANT-234 | 7143.054 | 7143.103 |
| ANT-235 | 7152.066 | 7152.084 |
| ANT-236 | 7177.072 | 7177.096 |
| ANT-237 | 7202.018 | 7202.036 |
| ANT-238 | 7159.049 | 7159.089 |
| ANT-239 | 7185.100 | 7185.103 |
| ANT-240 | 7218.073 | 7218.073 |
| ANT-241 | 7168.121 | 7168.143 |
| ANT-242 | 7222.070 | 7222.095 |
| ANT-243 | 7170.028 | 7170.045 |
| ANT-244 | 7179.040 | 7179.039 |
| ANT-245 | 7181.068 | 7181.091 |
| ANT-246 | 7168.060 | 7168.068 |
| ANT-247 | 7169.044 | 7169.045 |
| ANT-248 | 7240.033 | 7240.106 |
| ANT-249 | 7229.053 | 7229.106 |
| ANT-250 | 7142.070 | 7142.072 |
| ANT-251 | 7125.151 | 7125.147 |
| ANT-252 | 7156.097 | 7156.098 |
| ANT-253 | 7098.056 | 7098.053 |
| ANT-254 | 7181.103 | 7181.103 |
| ANT-255 | 7164.089 | 7164.112 |
| ANT-256 | 7175.069 | 7175.090 |
| ANT-257 | 7195.095 | 7195.114 |
| ANT-258 | 7255.104 | 7255.127 |
| ANT-263 | 7113.199 | 7113.263 |
| ANT-264 | 7145.153 | 7145.214 |
| ANT-265 | 7078.257 | 7078.318 |
| ANT-266 | 7110.211 | 7110.269 |
| ANT-267 | 7203.062 | 7203.069 |
| ANT-268 | 7168.000 | 7168.006 |
| ANT-269 | 7180.084 | 7180.088 |
| ANT-270 | 7262.027 | 7262.035 |
| ANT-271 | 7178.116 | 7178.122 |
| ANT-272 | 7170.028 | 7170.029 |
| ANT-273 | 7209.062 | 7209.057 |
| ANT-274 | 7139.118 | 7139.134 |
| ANT-275 | 7186.023 | 7186.020 |
| ANT-276 | 7102.053 | 7102.063 |
| ANT-277 | 7184.116 | 7184.120 |
| ANT-278 | 7133.119 | 7133.119 |
| ANT-279 | 7150.097 | 7150.099 |
| ANT-280 | 7251.047 | 7251.044 |
| ANT-281 | 7203.002 | 7202.998 |
| ANT-282 | 7128.103 | 7128.111 |
| ANT-283 | 7186.084 | 7186.093 |
| ANT-284 | 7226.041 | 7226.052 |
| ANT-285 | 7183.071 | 7183.076 |
| ANT-286 | 7131.090 | 7131.104 |
| ANT-287 | 7155.018 | 7155.028 |
| ANT-288 | 7250.088 | 7250.073 |
| ANT-289 | 7161.077 | 7161.083 |
| ANT-290 | 7183.036 | 7183.037 |
| ANT-291 | 7127.059 | 7127.069 |
| ANT-292 | 7123.183 | 7123.196 |
| ANT-293 | 7136.071 | 7136.077 |
| ANT-294 | 7162.121 | 7162.128 |
| ANT-295 | 7200.075 | 7200.085 |
| ANT-296 | 7228.069 | 7228.078 |
| ANT-297 | 7232.065 | 7232.075 |
| ANT-298 | 7231.081 | 7231.095 |
| ANT-299 | 7213.118 | 7213.134 |
| ANT-300 | 7258.056 | 7258.063 |
| ANT-301 | 7286.049 | 7286.063 |
| ANT-302 | 7248.060 | 7248.067 |
| ANT-303 | 7169.105 | 7169.100 |
| ANT-304 | 7169.105 | 7169.099 |
| ANT-305 | 7168.121 | 7168.113 |
| ANT-306 | 7184.116 | 7184.118 |
| ANT-307 | 7152.126 | 7152.129 |
| ANT-308 | 7161.077 | 7161.075 |
| ANT-309 | 7206.075 | 7206.059 |
| ANT-310 | 7197.063 | 7197.072 |
| ANT-311 | 7222.070 | 7222.083 |
| ANT-312 | 7212.074 | 7212.083 |
| ANT-313 | 7203.062 | 7203.050 |
| ANT-314 | 7212.074 | 7212.054 |
| ANT-315 | 7203.062 | 7203.042 |
| ANT-316 | 7204.046 | 7204.029 |
| ANT-317 | 7177.072 | 7177.060 |
| ANT-318 | 7168.060 | 7168.045 |
| ANT-319 | 7167.076 | 7167.071 |
| ANT-320 | 7193.067 | 7193.056 |
| ANT-321 | 7168.060 | 7168.051 |
| ANT-322 | 7158.065 | 7158.063 |
| ANT-323 | 7115.156 | 7115.144 |
| ANT-324 | 7157.141 | 7157.119 |
| ANT-325 | 7148.129 | 7148.117 |
| ANT-326 | 7174.120 | 7174.104 |
| ANT-327 | 7164.124 | 7164.109 |
| ANT-328 | 7165.108 | 7165.091 |
| ANT-329 | 7165.108 | 7165.094 |
| ANT-330 | 7173.076 | 7173.050 |
| ANT-331 | 7158.065 | 7158.046 |
| ANT-332 | 7142.070 | 7142.049 |
| ANT-333 | 7142.070 | 7142.052 |
| ANT-334 | 7142.070 | 7142.049 |
| ANT-335 | 7133.058 | 7133.034 |
| ANT-336 | 7133.058 | 7133.036 |
| ANT-337 | 7182.087 | 7182.066 |
| ANT-338 | 7182.087 | 7182.070 |
| ANT-339 | 7191.099 | 7191.083 |
| ANT-340 | 7190.115 | 7190.100 |
| ANT-341 | 7142.070 | 7142.055 |
| ANT-342 | 7158.065 | 7158.047 |
| ANT-343 | 7184.248 | 7184.250 |
| ANT-344 | 7168.271 | 7168.273 |
| ANT-345 | 7168.271 | 7168.269 |
| ANT-346 | 7140.222 | 7140.215 |
| ANT-347 | 7124.245 | 7124.244 |
| ANT-348 | 7124.245 | 7124.241 |
| ANT-349 | 7080.190 | 7080.186 |
| ANT-350 | 7103.168 | 7103.163 |
| ANT-351 | 7110.211 | 7110.209 |
| ANT-352 | 7078.161 | 7078.161 |
| ANT-353 | 7106.180 | 7106.180 |
| ANT-354 | 7138.170 | 7138.164 |
| ANT-359 | 7223.054 | 7223.019 |
| ANT-360 | 7159.109 | 7159.080 |
| ANT-361 | 7158.065 | 7158.038 |
| ANT-362 | 7166.092 | 7166.062 |
| ANT-363 | 7253.015 | 7252.986 |
| ANT-364 | 7212.014 | 7211.985 |
| ANT-365 | 7111.064 | 7111.038 |
| ANT-366 | 7160.093 | 7160.078 |
| ANT-367 | 7257.072 | 7257.056 |
| ANT-368 | 7206.075 | 7206.058 |
| ANT-369 | 7133.119 | 7133.099 |
| ANT-370 | 7136.010 | 7135.987 |
| ANT-371 | 7190.080 | 7190.042 |
| ANT-372 | 7145.082 | 7145.054 |
| ANT-373 | 7134.042 | 7134.015 |
| ANT-374 | 7150.037 | 7150.039 |
| ANT-375 | 7190.020 | 7190.027 |
| ANT-376 | 7189.096 | 7189.075 |
| ANT-377 | 7202.078 | 7202.053 |
| ANT-378 | 7180.024 | 7180.000 |
| ANT-379 | 7180.024 | 7180.000 |
| ANT-380 | 7136.010 | 7135.991 |
| ANT-381 | 7204.046 | 7204.046 |
| ANT-382 | 7168.060 | 7168.056 |
| ANT-383 | 7127.059 | 7127.022 |
| ANT-384 | 7185.100 | 7185.061 |
| ANT-385 | 7216.045 | 7216.009 |
| ANT-386 | 7259.075 | 7259.038 |
| ANT-387 | 7237.020 | 7236.983 |
| ANT-388 | 7194.975 | 7194.931 |
| ANT-389 | 7201.094 | 7201.057 |
| ANT-390 | 7203.062 | 7203.021 |
| ANT-391 | 7230.097 | 7230.050 |
| ANT-392 | 7135.147 | 7135.108 |
| ANT-393 | 7108.112 | 7108.123 |
| ANT-394 | 7190.080 | 7190.078 |
| ANT-395 | 7147.050 | 7147.056 |
| ANT-396 | 7091.013 | 7091.022 |
| ANT-397 | 7138.039 | 7138.039 |
| ANT-398 | 7111.064 | 7111.063 |
| ANT-399 | 7215.086 | 7215.085 |
| ANT-400 | 7195.035 | 7195.037 |
| ANT-401 | 7179.100 | 7179.101 |
| ANT-402 | 7204.107 | 7204.113 |
| ANT-403 | 7249.044 | 7249.049 |
| ANT-404 | 7281.034 | 7281.037 |
| ANT-405 | 7193.067 | 7193.072 |
| ANT-406 | 7195.095 | 7195.092 |
| ANT-407 | 7104.056 | 7104.055 |
| ANT-408 | 7137.090 | 7137.084 |
| ANT-409 | 7154.034 | 7154.030 |
| ANT-410 | 7067.182 | 7067.168 |
| ANT-411 | 7093.232 | 7093.230 |
| ANT-423 | 7188.052 | 7188.063 |
| ANT-424 | 7178.056 | 7178.074 |
| ANT-425 | 7168.060 | 7168.073 |
| ANT-426 | 7176.088 | 7176.099 |
| ANT-427 | 7186.084 | 7186.094 |
| ANT-428 | 7195.095 | 7195.116 |
| ANT-429 | 7247.076 | 7247.092 |
| ANT-430 | 7237.081 | 7237.093 |
| ANT-431 | 7237.081 | 7237.091 |
| ANT-432 | 7212.074 | 7212.085 |
| ANT-433 | 7196.079 | 7196.089 |
| ANT-434 | 7196.079 | 7196.093 |
| ANT-435 | 7210.046 | 7210.057 |
| ANT-436 | 7210.046 | 7210.060 |
| ANT-437 | 7218.073 | 7218.093 |
| ANT-438 | 7192.083 | 7192.098 |
| ANT-439 | 7193.067 | 7193.083 |
| ANT-440 | 7141.146 | 7141.158 |
| ANT-441 | 7157.141 | 7157.155 |
| ANT-442 | 7148.129 | 7148.143 |
| ANT-443 | 7148.129 | 7148.140 |
| ANT-444 | 7174.120 | 7174.124 |
| ANT-445 | 7156.097 | 7156.108 |
| ANT-446 | 7156.097 | 7156.107 |
| ANT-447 | 7172.092 | 7172.096 |
| ANT-448 | 7182.087 | 7182.095 |
| ANT-449 | 7114.111 | 7114.118 |
| ANT-450 | 7105.100 | 7105.109 |
| ANT-451 | 7105.100 | 7105.110 |
| ANT-452 | 7122.079 | 7122.085 |
| ANT-453 | 7123.063 | 7123.076 |
| ANT-454 | 7132.074 | 7132.074 |
| ANT-455 | 7142.070 | 7142.082 |
| ANT-456 | 7150.037 | 7150.048 |
| ANT-457 | 7151.021 | 7151.027 |
| ANT-458 | 7135.026 | 7135.034 |
| ANT-459 | 7164.089 | 7164.100 |
| ANT-460 | 7154.094 | 7154.097 |
| ANT-461 | 7154.094 | 7154.100 |
| ANT-462 | 7161.077 | 7161.084 |
| ANT-463 | 7161.077 | 7161.085 |
| ANT-464 | 7145.082 | 7145.093 |
| ANT-465 | 7237.020 | 7237.028 |
| ANT-466 | 7237.020 | 7237.034 |
| ANT-467 | 7227.025 | 7227.033 |
| ANT-468 | 7228.009 | 7228.019 |
| ANT-469 | 7203.002 | 7203.009 |
| ANT-470 | 7212.998 | 7213.004 |
| ANT-471 | 7169.105 | 7169.109 |
| ANT-472 | 7185.100 | 7185.100 |
| ANT-473 | 7195.095 | 7195.100 |
| ANT-474 | 7195.095 | 7195.099 |
| ANT-475 | 7101.200 | 7101.213 |
| ANT-476 | 7214.042 | 7214.054 |
| ANT-477 | 7228.009 | 7228.017 |
| ANT-478 | 7212.998 | 7213.002 |
| ANT-479 | 7247.076 | 7247.084 |
| ANT-480 | 7167.076 | 7167.069 |
| ANT-481 | 7167.076 | 7167.081 |
| ANT-482 | 7168.060 | 7168.064 |
| ANT-483 | 7132.074 | 7132.079 |
| ANT-484 | 7129.062 | 7129.065 |
| ANT-485 | 7113.067 | 7113.067 |
| ANT-486 | 7180.084 | 7180.089 |
| ANT-487 | 7101.217 | 7101.220 |
| ANT-488 | 7101.217 | 7101.237 |
| ANT-489 | 7092.195 | 7092.194 |
| ANT-490 | 7092.195 | 7092.210 |
| ANT-491 | 7103.168 | 7103.169 |
| ANT-492 | 7103.185 | 7103.187 |
| ANT-493 | 7103.185 | 7103.207 |
| ANT-494 | 7189.107 | 7189.111 |
| ANT-495 | 7203.140 | 7203.143 |
| ANT-496 | 7203.140 | 7203.160 |
| ANT-497 | 7164.160 | 7164.157 |
| ANT-498 | 7164.178 | 7164.180 |
| ANT-499 | 7164.178 | 7164.195 |
| ANT-500 | 7094.156 | 7094.153 |
| ANT-501 | 7108.190 | 7108.188 |
| ANT-502 | 7108.190 | 7108.211 |
| ANT-503 | 7112.179 | 7112.176 |
| ANT-504 | 7112.197 | 7112.228 |
| ANT-505 | 7112.197 | 7112.216 |
| ANT-506 | 7173.172 | 7173.188 |
| ANT-507 | 7187.205 | 7187.237 |
| ANT-508 | 7187.205 | 7187.222 |
| ANT-509 | 7172.080 | 7172.068 |
| ANT-510 | 7156.103 | 7156.089 |
| ANT-511 | 7156.103 | 7156.093 |
| ANT-512 | 7095.200 | 7095.199 |
| ANT-513 | 7106.180 | 7106.177 |
| ANT-514 | 7173.172 | 7173.171 |
| ANT-515 | 7108.172 | 7108.174 |
| ANT-516 | 7093.250 | 7093.247 |
| ANT-517 | 7093.250 | 7093.250 |
| ANT-518 | 7122.175 | 7122.169 |
| ANT-519 | 7122.193 | 7122.189 |
| ANT-520 | 7122.193 | 7122.189 |
| ANT-521 | 7095.218 | 7095.217 |
| ANT-522 | 7095.218 | 7095.219 |
| ANT-523 | 7077.237 | 7077.243 |
| ANT-524 | 7071.178 | 7071.179 |
| ANT-525 | 7071.196 | 7071.200 |
| ANT-526 | 7071.196 | 7071.191 |
| ANT-527 | 7187.205 | 7187.214 |
| ANT-528 | 7187.205 | 7187.209 |
| ANT-529 | 7093.232 | 7093.240 |

1. An antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 1 to 102, 159 to 842, 879 to 1822, and 1874 to 4182 from the 5′ end of a base sequence of SEQ ID NO: 1,
wherein an antisense oligonucleotide or a pharmaceutically acceptable salt thereof, or a hydrate thereof having a sequence consisting of a base sequence selected from the group consisting of SEQ ID NOs: 65, 436 to 440, 444 to 449, 451 to 452, and 454 to 474 is excluded, and
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is excluded, and
an antisense oligonucleotide having a sequence consisting of a base sequence of SEQ ID NO: 453 or a pharmaceutically acceptable salt thereof, or a hydrate thereof, in which positions 1 to 2, 4, 6, 8 to 9, 11 to 12, and 16 to 20 from the 5′ end of the base sequence of SEQ ID NO: 453 are ENA: 2′-O,4′-C-ethylene bridged nucleic acid, and positions 3, 5, 7, 10, and 13 to 15 are ribonucleotides comprising a 2′-OMe group, is excluded.

2. The antisense oligonucleotide or the pharmaceutically acceptable salt thereof, or the hydrate thereof according to claim 1, comprising:
(i) a base sequence selected from the group consisting of SEQ ID NOs: 2 to 64, 66 to 224, 422 to 423, 426 to 427, and 432;
(ii) a base sequence having addition, deletion, or substitution of one or several bases in a base sequence selected from the group consisting of SEQ ID NOs: 2 to 64, 66 to 224, 422 to 423, 426 to 427, and 432; or
(iii) a base sequence having 90% or more sequence identity with a base sequence selected from the group consisting of SEQ ID NOs: 2 to 64, 66 to 224, 422 to 423, 426 to 427, and 432.

3. The antisense oligonucleotide or the pharmaceutically acceptable salt thereof, or the hydrate thereof according to claim 1, complementary to a nucleic acid comprising at least 15 consecutive bases in a target region selected from the group consisting of positions 1 to 102, 159 to 622, 639 to 842, 879 to 1442, 1459 to 1497, 1499 to 1522, 1539 to 1702, 1719 to 1762, 1779 to 1802, 1874 to 1937, 1939 to 2302, 2319 to 3162, 3199 to 3242, 3279 to 3382, 3399 to 3482, 3539 to 3562, 3579 to 3602, 3619 to 3662, 3679 to 3702, and 3759 to 4182 from the 5′ end of the base sequence of SEQ ID NO: 1.

4. The antisense oligonucleotide or the pharmaceutically acceptable salt thereof, or the hydrate thereof according to claim 3, comprising:
(i) a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224;
(ii) a base sequence having addition, deletion, or substitution of one or several bases in a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224; or
(iii) a base sequence having 90% or more sequence identity with a base sequence selected from the group consisting of SEQ ID NOs: 2 to 30, 32 to 64, 66 to 72, 74 to 75, 77, 79 to 86, 88 to 89, 91, 93 to 96, 98 to 102, 104 to 120, 122 to 146, 148 to 155, 157 to 158, 160 to 173, 176 to 177, 180 to 184, 186 to 189, 193, 195, 197 to 198, 200, and 204 to 224.

5. The antisense oligonucleotide or the pharmaceutically acceptable salt thereof, or the hydrate thereof according to claim 1, complementary to a nucleic acid comprising at least 15 consecutive bases in a target region selected from the group consisting of positions 19 to 42, 79 to 102, 159 to 182, 199 to 622, 639 to 662, 679 to 762, 779 to 802, 819 to 842, 879 to 1002, 1019 to 1402, 1419 to 1442, 1459 to 1497, 1499 to 1522, 1559 to 1642, 1659 to 1702, 1779 to 1802, 1874 to 1902, 1914 to 1937, 1959 to 2002, 2019 to 2302, 2319 to 2342, 2359 to 2802, 2819 to 3142, 3199 to 3222, 3299 to 3322, 3339 to 3362, 3399 to 3422, 3799 to 3842, 3879 to 3982, 4019 to 4042, and 4119 to 4142 from the 5′ end of the base sequence of SEQ ID NO: 1.

6. The antisense oligonucleotide or the pharmaceutically acceptable salt thereof, or the hydrate thereof according to claim 5, comprising:
(i) a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222;
(ii) a base sequence having addition, deletion, or substitution of one or several bases in a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222; or
(iii) a base sequence having 90% or more sequence identity with a base sequence selected from the group consisting of SEQ ID NOs: 3, 6 to 7, 9 to 30, 32, 34 to 37, 39, 41 to 48, 50 to 64, 66 to 69, 72, 74 to 75, 77, 80 to 83, 85 to 86, 91, 93 to 94, 96, 99 to 100, 102, 104 to 120, 122, 124 to 143, 145 to 146, 148 to 149, 151, 154 to 155, 157 to 158, 160 to 165, 167 to 172, 176, 181, 183, 186, 206 to 207, 210 to 214, 217, and 222.

7. The antisense oligonucleotide or the pharmaceutically acceptable salt thereof, or the hydrate thereof according to claim 1, complementary to a nucleic acid comprising at least 15 consecutive bases in a target region selected from the group consisting of positions 19 to 42, 159 to 182, 239 to 282, 319 to 342, 359 to 482, 499 to 602, 639 to 662, 679 to 762, 779 to 802, 819 to 842, 879 to 902, 919 to 1002, 1039 to 1342, 1359 to 1382, 1419 to 1442, 1459 to 1482, 1499 to 1522, 1559 to 1642, 1659 to 1702, 1779 to 1802, 1879 to 1902, 1914 to 1937, 1979 to 2002, 2019 to 2042, 2059 to 2302, 2319 to 2342, 2359 to 2422, 2446 to 2542, 2559 to 2622, 2639 to 2702, 2939 to 2962, 3019 to 3042, 3119 to 3142, and 3199 to 3222 from the 5′ end of the base sequence of SEQ ID NO: 1.

8. The antisense oligonucleotide or the pharmaceutically acceptable salt thereof, or the hydrate thereof according to claim 7, comprising:
(i) a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176;
(ii) a base sequence having addition, deletion, or substitution of one or several bases in a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176; or
(iii) a base sequence having 90% or more sequence identity with a base sequence selected from the group consisting of SEQ ID NOs: 3, 7, 11 to 12, 15, 17 to 22, 24 to 29, 32, 34 to 37, 39, 41 to 42, 44 to 48, 51 to 64, 66, 68, 72, 74, 77, 80 to 83, 85 to 86, 91, 94, 96, 100, 102, 105 to 120, 122, 124 to 126, 129 to 133, 136 to 138, 140, 142 to 143, 162, 167, 172, and 176.

9. (canceled)

10. The antisense oligonucleotide or the pharmaceutically acceptable salt thereof, or the hydrate thereof according to claim 1, comprising:
(i) a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142;
(ii) a base sequence having addition, deletion, or substitution of one or several bases in a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142; or
(iii) a base sequence having 90% or more sequence identity with a base sequence selected from the group consisting of SEQ ID NOs: 11, 17 to 18, 20 to 21, 25, 27 to 29, 35, 41 to 42, 44, 46 to 47, 54 to 55, 57, 59, 62, 64, 66, 68, 74, 77, 80 to 81, 85, 91, 94, 100, 102, 105 to 108, 110 to 114, 116, 118 to 120, 129, 131, and 142.

11. (canceled)

12. An antisense oligonucleotide or a pharmaceutically acceptable salt thereof, or a hydrate thereof, consisting of 15 to 22 nucleotides complementary to a nucleic acid comprising at least 15 consecutive bases in a target region selected from the group consisting of positions 194 to 217, 350 to 452, 500 to 612, 629 to 684, 689 to 732, 821 to 844, 874 to 972, 1089 to 1177, 1277 to 1338, 1349 to 1392, 1444 to 1537, 1544 to 1592, 1644 to 1697, 1769 to 1812, 1969 to 2012, 2051 to 2317, and 2469 to 2512 from the 5′ end of a base sequence of SEQ ID NO: 1,
wherein an antisense oligonucleotide or a pharmaceutically acceptable salt thereof, or a hydrate thereof having a sequence consisting of a base sequence selected from the group consisting of SEQ ID NOs: 436 to 438, 440 to 449, 451 to 452, 454 to 455, and 472 to 474 is excluded, and
an antisense oligonucleotide having a sequence consisting of a base sequence of SEQ ID NO: 450 or a pharmaceutically acceptable salt thereof, or a hydrate thereof, in which positions 2 to 3, 5 to 6, 10 to 14, 16, 18 and 20 from the 5′ end of the base sequence of SEQ ID NO: 450 are ENA: 2′-O,4′-C-ethylene bridged nucleic acid, and positions 1, 4, 7 to 9, 15, 17, and 19 are ribonucleotides comprising a 2′-OMe group, is excluded, and
an antisense oligonucleotide having a sequence consisting of a base sequence of SEQ ID NO: 453 or a pharmaceutically acceptable salt thereof, or a hydrate thereof, in which positions 1 to 2, 4, 6, 8 to 9, 11 to 12, and 16 to 20 from the 5′ end of the base sequence of SEQ ID NO: 453 are ENA: 2′-O,4′-C-ethylene bridged nucleic, and positions 3, 5, 7, 10, and 13 to 15 are ribonucleotides comprising a 2′-OMe group, is excluded.

13. The antisense oligonucleotide or the pharmaceutically acceptable salt thereof, or the hydrate thereof according to claim 12, comprising:
(i) a base sequence selected from the group consisting of SEQ ID NOs: 231 to 419, 424 to 425, 428 to 431, 433 to 435, and 475 to 502;
(ii) a base sequence having addition, deletion, or substitution of one or several bases in a base sequence selected from the group consisting of SEQ ID NOs: 231 to 419, 424 to 425, 428 to 431, 433 to 435, and 475 to 502; or
(iii) a base sequence having 90% or more sequence identity with a base sequence selected from the group consisting of SEQ ID NOs: 231 to 419, 424 to 425, 428 to 431, 433 to 435, and 475 to 502.

14. The antisense oligonucleotide or the pharmaceutically acceptable salt thereof, or the hydrate thereof according to claim 12, complementary to a nucleic acid comprising at least 15 consecutive bases in a target region selected from the group consisting of positions 194 to 217, 350 to 452, 500 to 612, 629 to 679, 689 to 732, 821 to 844, 874 to 912, 924 to 972, 1089 to 1177, 1277 to 1338, 1349 to 1392, 1449 to 1484, 1504 to 1532, 1544 to 1592, 1644 to 1697, 1769 to 1812, 1969 to 2012, 2051 to 2252, 2264 to 2317, and 2476 to 2512 from the 5′ end of the base sequence of SEQ ID NO: 1.

15. The antisense oligonucleotide or the pharmaceutically acceptable salt thereof, or the hydrate thereof according to claim 14, comprising:
(i) a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502;
(ii) a base sequence having addition, deletion, or substitution of one or several bases in a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502; or
(iii) a base sequence having 90% or more sequence identity with a base sequence selected from the group consisting of SEQ ID NOs: 231 to 242, 244, 246 to 268, 270 to 271, 273 to 285, 288, 290 to 292, 296 to 297, 299 to 390, 392 to 408, 412 to 417, 419, 475 to 476, 478 to 497, and 499 to 502.

16. The antisense oligonucleotide or the pharmaceutically acceptable salt thereof, or the hydrate thereof according to claim 12, complementary to a nucleic acid comprising at least 15 consecutive bases in a target region selected from the group consisting of positions 351 to 376, 384 to 452, 509 to 552, 569 to 607, 656 to 679, 821 to 844, 874 to 912, 927 to 952, 1109 to 1177, 1277 to 1338, 1364 to 1392, 1449 to 1484, 1504 to 1532, 1544 to 1587, 1646 to 1697, 1774 to 1812, 1984 to 2007, 2054 to 2127, 2131 to 2215, 2229 to 2252, 2264 to 2312, and 2478 to 2512 from the 5′ end of the base sequence of SEQ ID NO: 1.

17. The antisense oligonucleotide or the pharmaceutically acceptable salt thereof, or the hydrate thereof according to claim 16, comprising:
(i) a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502;
(ii) a base sequence having addition, deletion, or substitution of one or several bases in a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502; or
(iii) a base sequence having 90% or more sequence identity with a base sequence selected from the group consisting of SEQ ID NOs: 232 to 234, 236 to 238, 240 to 241, 244, 247 to 255, 258, 262 to 264, 268, 274 to 285, 290, 292, 296 to 297, 299 to 300, 302, 306 to 312, 314 to 316, 318 to 320, 323, 328 to 340, 342 to 350, 353 to 359, 361 to 377, 380 to 382, 384 to 387, 390, 392 to 399, 402 to 407, 414 to 415, 419, 478, 482 to 483, 485 to 487, 489 to 496, and 499 to 502.

18. The antisense oligonucleotide or the pharmaceutically acceptable salt thereof, or the hydrate thereof according to claim 12, complementary to a nucleic acid comprising at least 15 consecutive bases in a target region selected from the group consisting of positions 409 to 452, 509 to 548, 656 to 679, 874 to 907, 927 to 950, 1129 to 1172, 1294 to 1333, 1449 to 1484, 1504 to 1532, 1549 to 1587, 1646 to 1697, 1784 to 1807, 2057 to 2117, 2131 to 2215, 2266 to 2312, and 2478 to 2501 from the 5′ end of the base sequence of SEQ ID NO: 1.

19. The antisense oligonucleotide or the pharmaceutically acceptable salt thereof, or the hydrate thereof according to claim 18, comprising:
(i) a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502;
(ii) a base sequence having addition, deletion, or substitution of one or several bases in a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502; or
(iii) a base sequence having 90% or more sequence identity with a base sequence selected from the group consisting of SEQ ID NOs: 240 to 241, 244, 249 to 252, 254 to 255, 268, 275, 277 to 281, 284, 290, 292, 296 to 297, 300, 302, 306 to 312, 315 to 316, 319, 329 to 331, 334 to 339, 343 to 349, 353 to 359, 361 to 365, 367 to 371, 375 to 377, 381 to 382, 384, 386 to 387, 393 to 394, 396 to 399, 402 to 405, 407, 414, 478, 482, 487, 489, 490, 493, 495, and 502.

20. (canceled)

21. The antisense oligonucleotide or the pharmaceutically acceptable salt thereof, or the hydrate thereof according to claim 12, comprising:
(i) a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490;
(ii) a base sequence having addition, deletion, or substitution of one or several bases in a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490; or
(iii) a base sequence having 90% or more sequence identity with a base sequence selected from the group consisting of SEQ ID NOs: 250 to 251, 254 to 255, 280, 292, 297, 300, 307 to 310, 316, 319, 335 to 337, 343, 346, 349, 353 to 356, 359, 361 to 364, 367, 371, 375, 377, 381 to 382, 384, 387, 396 to 399, 402 to 403, 405, 482, 487, 489, and 490.

22. (canceled)

23. The antisense oligonucleotide or the pharmaceutically acceptable salt thereof, or the hydrate thereof according to claim 12, comprising:
(i) a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402;
(ii) a base sequence having addition, deletion, or substitution of one or several bases in a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402; or
(iii) a base sequence having 90% or more sequence identity with a base sequence selected from the group consisting of SEQ ID NOs: 25, 42, 74, 80, 105 to 107, 112, 119, 131, 250 to 251, 254, 307 to 309, 316, 337, 346, 354 to 356, 363 to 364, 371, 381, 387, 396, 398 to 399, and 402.

24.-39. (canceled)

40. A method for treating and/or preventing a TDP-43 proteinopathy, comprising a step of administering, to a subject, the antisense oligonucleotide or the pharmaceutically acceptable salt thereof, or the hydrate thereof according to claim 1.
